(19) United States
(12) Patent Application Publication
(10) Pub. No.: US 2025/0048920 A1
(43) Pub. Date: Feb. 6, 2025

(54) COMPOUND FOR ORGANIC ELECTRIC ELEMENT, ORGANIC ELECTRIC ELEMENT USING THE SAME, AND ELECTRONIC DEVICE THEREOF

(71) Applicant: DUK SAN NEOLUX CO., LTD., Cheonan-si (KR)

(72) Inventors: Yeong Ran SONG, Cheonan-si (KR); Junggeun LEE, Cheonan-si (KR); Sun-Hee LEE, Cheonan-si (KR)

(73) Assignee: DUK SAN NEOLUX CO., LTD., Cheonan-si (KR)

(21) Appl. No.: 18/922,933

(22) Filed: Oct. 22, 2024

Related U.S. Application Data
(63) Continuation-in-part of application No. 17/230,061, filed on Apr. 14, 2021, now Pat. No. 12,178,118.
Application No. 17/230,061 is a continuation of application No. 17/212,776, filed on Mar. 25, 2021, now Pat. No. 12,041,847.
Application No. 17/212,776 is a continuation of application No. 16/344,684, filed on Apr. 24, 2019, now Pat. No. 11,910,705.

(30) Foreign Application Priority Data
Feb. 2, 2018 (KR) 10-2018-0013219

(51) Int. Cl. H10K 85/60; C07D 209/86; C07D 333/76; C07D 403/10; C07D 403/14; C07D 405/14; C07D 409/14; C09K 11/06; H10K 50/11; H10K 50/15; H10K 50/18; H10K 50/85; H10K 71/00; H10K 71/16; H10K 101/00; H10K 101/10; H10K 101/40; H10K 102/00

(52) U.S. Cl. CPC H10K 85/633; C07D 209/86; C07D 333/76; C07D 403/10; C07D 403/14; C07D 405/14; C07D 409/14; C09K 11/06; H10K 50/18; H10K 85/626; H10K 85/636; H10K 85/654; H10K 85/6572; H10K 85/6574; H10K 85/6576; C09K 2211/1018; H10K 50/11; H10K 50/15; H10K 50/85; H10K 71/00; H10K 71/164; H10K 2101/10; H10K 2101/40; H10K 2101/90; H10K 2102/351

(57) ABSTRACT

Provided are a compound of Formula 37 capable of improving luminous efficiency, stability and lifetime of an organic electronic element employing the same, an organic electronic element using the same, and an electronic device thereof.

### BACKGROUND

### Technical Field

The present invention relates to compound for organic electric element, organic electric element using the same, and an electronic device thereof.

### Background Art

In general, organic light emitting phenomenon refers to a phenomenon that converts electric energy into light energy by using an organic material. An organic electric element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electric element may be classified into a light emitting material and a charge transport material, such as a hole injection material, a hole transport material, an electron transport material, an electron injection material and the like depending on its function.

And the light emitting material may be classified into a polymer type and a low molecular type depending on the molecular weight, and into a fluorescent material derived from the singlet excited state of electrons and a phosphorescent material derived from the triplet excited state of electrons depending on the light emitting mechanism. Further, the light emitting material can be classified into blue, green, and red light emitting materials and yellow and orange light emitting materials necessary for realizing better natural color depending on the luminescent color.

Meanwhile, when only one material is used as a light emitting material, there arises a problem that the maximum light emission wavelength shifts to a long wavelength due to intermolecular interaction, the color purity drops, or the efficiency of the device decreases due to the light emission attenuation effect, therefore a host/dopant system can be used as a light emitting material in order to increase luminous efficiency through increase of color purity and energy transfer. When the small amount of dopant having a smaller energy band gap than the host forming the emitting layer is mixed on the emitting layer, the excitons generated in the emitting layer are transported to the dopant to emit light with high efficiency. At this time, since the wavelength of the host is shifted to the wavelength band of the dopant, light of a desired wavelength can be obtained depending on the type of the dopant used.

Currently, the portable display market is growing in size as a large-area display, which requires more power than the power consumption required by existing portable displays. Therefore, power consumption is a very important factor for portable displays, which have a limited power source, such as a battery, and efficiency and lifetime issues must be solved.

Efficiency, life span, driving voltage and the like are related to each other. As the efficiency is increased, the driving voltage is relatively decreased, and as the driving voltage drops, the crystallization of the organic material due to joule heating generated during driving is reduced, and as a result, the life span tends to increase. However, simply improving the organic material layer cannot maximize the efficiency. This is because, when the optimal combination of the energy level and T1 value between each organic material layer and the intrinsic properties (mobility, interface characteristics, etc.) of the material are achieved, long life and high efficiency can be achieved at the same time. Therefore, it is necessary to develop a light emitting material having a high thermal stability and achieving a charge balance in the emitting layer efficiently.

That is, in order to sufficiently exhibit the excellent characteristics of the organic electric element, a material for forming an organic material layer in an element such as a hole injection material, a hole transport material, a light emitting material, an electron transport material, an electron injection material, an emitting-auxiliary layer material, and the like should be supported by stable and efficient materials. However, such a stable and efficient organic material layer material for an organic electric element has not been sufficiently developed yet. Therefore, development of new materials is continuously required, and especially development of host materials for the emitting layer is urgently required.

Otherwise, in the case of a polycyclic compound including a heteroatom, the difference in properties according to the material structure is so large that it is applied to various layers as OLED material. In particular, it has characteristics of different band gaps (HOMO, LUMO), electrical characteristics, chemical properties, and physical properties depending on the number of rings, fused positions and the type and arrangement of heteroatoms, therefore application development for various OLED layers using the same has been progressed. Recently, development of OLED material for heteroatom type, number and position of pentacyclic compounds has been actively developed.

As a precedent reference, U.S. Pat. No. 8,334,058 B2 is referred to.

### DETAILED DESCRIPTION OF THE INVENTION

### Summary

Using the characteristics of the polycyclic compound, the present invention provides a compound capable of maximizing the effect of improving luminous efficiency and long life, while maintaining or slightly reducing the driving voltage of the device, and an organic electric element using the same and an electronic device thereof.

### Technical Solution

The present invention provides compounds represented by Formula (37), organic electric elements comprising the same and electronic devices thereof.

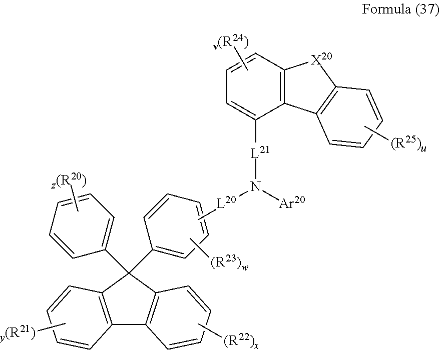

### Effects of the Invention

By using the compound according to the present invention, it is possible to achieve a high luminous efficiency, a low driving voltage, and a high heat resistance of the element, and can greatly improve the color purity and lifetime of the element.

### BRIEF DESCRIPTION OF THE DRAWINGS

The FIGURE illustrates an example of an organic electric element according to the present invention.

| | |
|---|---|
| 100: organic electric element, | 110: substrate |
| 120: the first electrode(anode), | 130: the hole injection layer |
| 140: the hole transport layer, | 141: a buffer layer |
| 150: the emitting layer, | 151: the emitting auxiliary layer |
| 160: the electron transport layer, | 170: the electron injection layer |
| 180: the second electrode(cathode) | |

### DETAILED DESCRIPTION OF THE INVENTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected “,” coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “haloalkyl” or “halogen alkyl”, as used herein, includes an alkyl group substituted with a halogen.

Unless otherwise stated, the term “heteroalkyl”, as used herein, means alkyl substituted one or more of carbon atoms consisting of an alkyl with heteroatom.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “alkenoxyl group”, “alkenoxy group”, “alkenyloxyl group” or “alkenyloxy group”, as used herein, means an oxygen radical attached to an alkenyl group, but is not limited thereto, and has 2 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

Unless otherwise stated, the term “aryl group” or “arylene group”, as used herein, has 6 to 60 carbon atoms, but is not limited thereto. Herein, the aryl group or arylene group means a monocyclic and polycyclic aromatic group, and may also be formed in conjunction with an adjacent group. Examples of “aryl group” may include a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heteroalkyl”, as used herein, means alkyl including one or more of heteroatoms. Unless otherwise stated, the term “heteroaryl group” or “heteroarylene group”, as used herein, means a C2 to C60 aryl including one or more of heteroatoms or arylene group, but is not limited thereto, and includes at least one of monocyclic and polycyclic rings, and may also be formed in conjunction with an adjacent group.

Unless otherwise stated, the term “heterocyclic group”, as used herein, contains one or more heteroatoms, but is not limited thereto, has 2 to 60 carbon atoms, includes any one of monocyclic and polycyclic rings, and may include heteroaliphadic ring and/or heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes compound below.

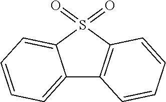 [SMILES: O=S1(=O)c2ccccc2-c2ccccc21]

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Unless otherwise stated, the term “carbonyl”, as used herein, is represented by —COR′, wherein R′ may be hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “ether”, as used herein, is represented by —R—O—R′, wherein R or R′ may be independently hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “substituted or unsubstituted”, as used herein, means that substitution is substituted by at least one substituent selected from the group consisting of, but is not limited thereto, deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group.

Unless otherwise expressly stated, the Formula used in the present invention, as used herein, is applied in the same manner as the substituent definition according to the definition of the exponent of the following Formula.

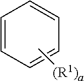

Wherein, when a is an integer of zero, it means the substituent R¹ is absent. That is, when a is 0, it means that all the carbons forming the benzene ring are bonded to hydrogen. In this case, the sign of the hydrogen bonded to the carbon may be omitted and the formula or compound may be described. When a is an integer of 1, the sole substituent R¹ is linked to any one of the carbon constituting the benzene ring, when a is an integer of 2 or 3, they are respectively bonded as follows, in which R¹ may be the same as or different from each other, and when a is an integer of 4 to 6, and it is bonded to the carbon of the benzene ring in a similar manner, whereas the indication of hydrogen bonded to the carbon forming the benzene ring is omitted.

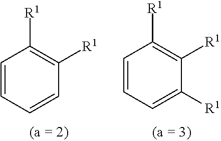

Hereinafter, a compound according to an aspect of the present invention and an organic electric element comprising the same will be described.

According to a specific example of the present invention, there is provided a compound represented by Formula (37).

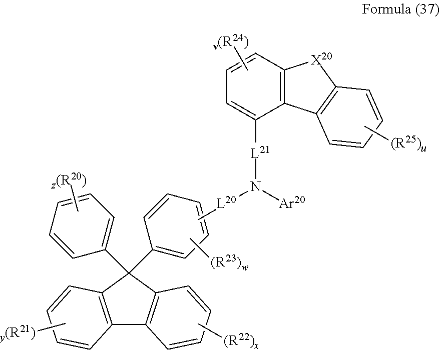

As another example, the present invention provides an organic electronic element comprising an anode; a cathode; an organic material layer formed between the anode and the cathode; wherein the organic material layer includes an emitting layer, an hole transport layer formed between the anode and the emitting layer; an emitting auxiliary layer formed between the emitting layer and the hole transport layer; wherein the hole transport layer or the emitting auxiliary layer comprises a compound represented by Formula (18).

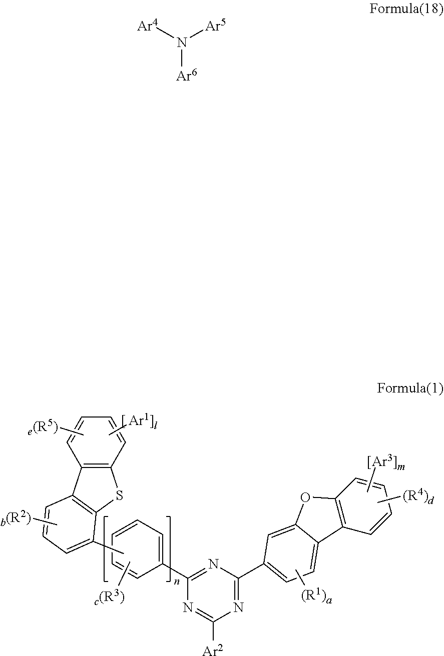

In Formula (18),
Ar⁴ and Ar⁵ are each independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₅₀ alkoxyl group; a C₆-C₃₀ aryloxy group; and -L′-N(Rₐ)(R_{b});
wherein, L′ may be selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic group;
and the Rₐ and R_{b} are each independently selected from the group consisting of a C₆-C₆₀ aryl group; a fluorenyl group; a fused ring group of a C₃-C₆₀ aliphatic ring and a C₆-C₆₀ aromatic ring; and a C₂-C₆₀ heterocyclic group including at least one heteroatom of O, N, S, Si, or P;
or Ar⁴ and Ar⁵ may be bonded to each other to form a ring.

Ar⁶ is selected from the group consisting of a C₆-C₆₀ aryl group; a fluorenyl group; a C₂-C₆₀ heterocyclic group including at least one heteroatom of O, N, S, Si, or P; or is at least one of the following Formulas (1-a), (1-b), (1-c)

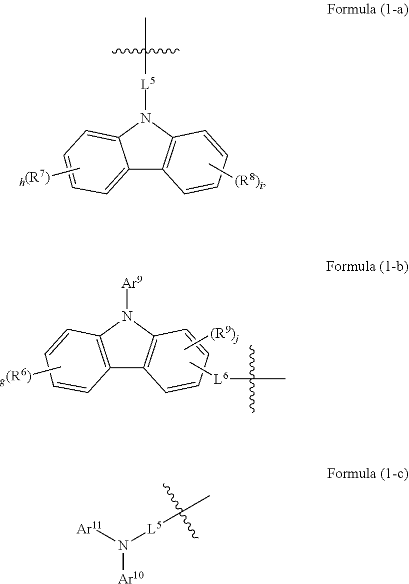

Ar⁹, Ar¹⁰ and Ar¹¹ are each independently selected from the group consisting of a C₆-C₆₀ aryl group; a fluorenyl group; a C₂-C₆₀ heterocyclic group including at least one heteroatom of O, N, S, Si, or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b});
h, i and g are an integer of 0 to 4; j is an integer of 0 to 3; R⁶, R⁷, R⁸ and R⁹ are the same or different from each other, and are each independently selected from the group consisting of hydrogen; deuterium; halogen; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b});
wherein in case g, h, i, j are 2 or more, R⁶, R⁷, R⁸ and R⁹ are each in plural being the same or different, and may be bonded to each other to form a ring,
L⁶ is selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic group including at least one heteroatom of O, N, S, Si or P;
L⁵ is selected from the group consisting of a C₆-C₆₀ arylene group; a fluorenylene group; a fused ring group of a C₃-C₆₀ aliphatic ring and a C₆-C₆₀ aromatic ring; and a C₂-C₆₀ heterocyclic group including at least one heteroatom of O, N, S, Si or P.

Also, the hole transport layer comprises a compound represented by Formula (19) or Formula (20), and the emitting auxiliary layer comprises a compound represented by Formula (21) or Formula (22)

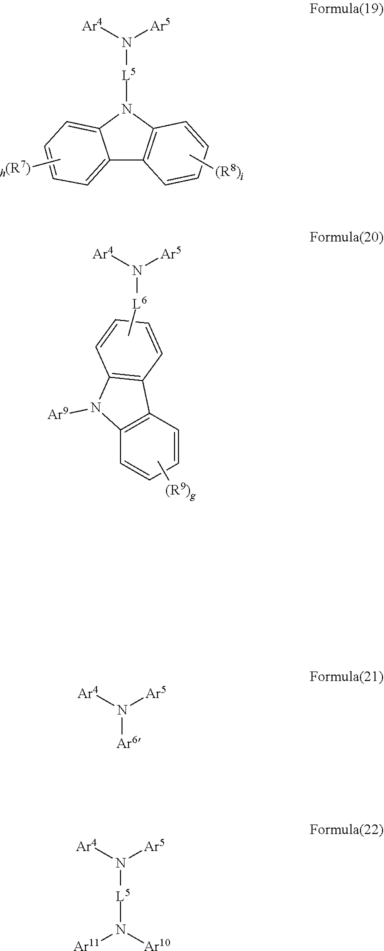

In Formulas (19) to (22),
1) Ar⁶ is selected from the group consisting of a C₆-C₆₀ aryl group; a fluorenyl group; a C₂-C₆₀ heterocyclic group including at least one heteroatom of O, N, S, Si or P;
2) Ar⁴, Ar⁵, Ar⁹, Ar¹⁰, Ar¹¹, h, i, g, L⁵, L⁶, R⁶, R⁷, R⁸ and R⁹ are the same as defined above.

Specifically, the compound represented by Formula (18) comprises the following compounds 13-1 to 13-79 and compounds 2-1 to 2-187.

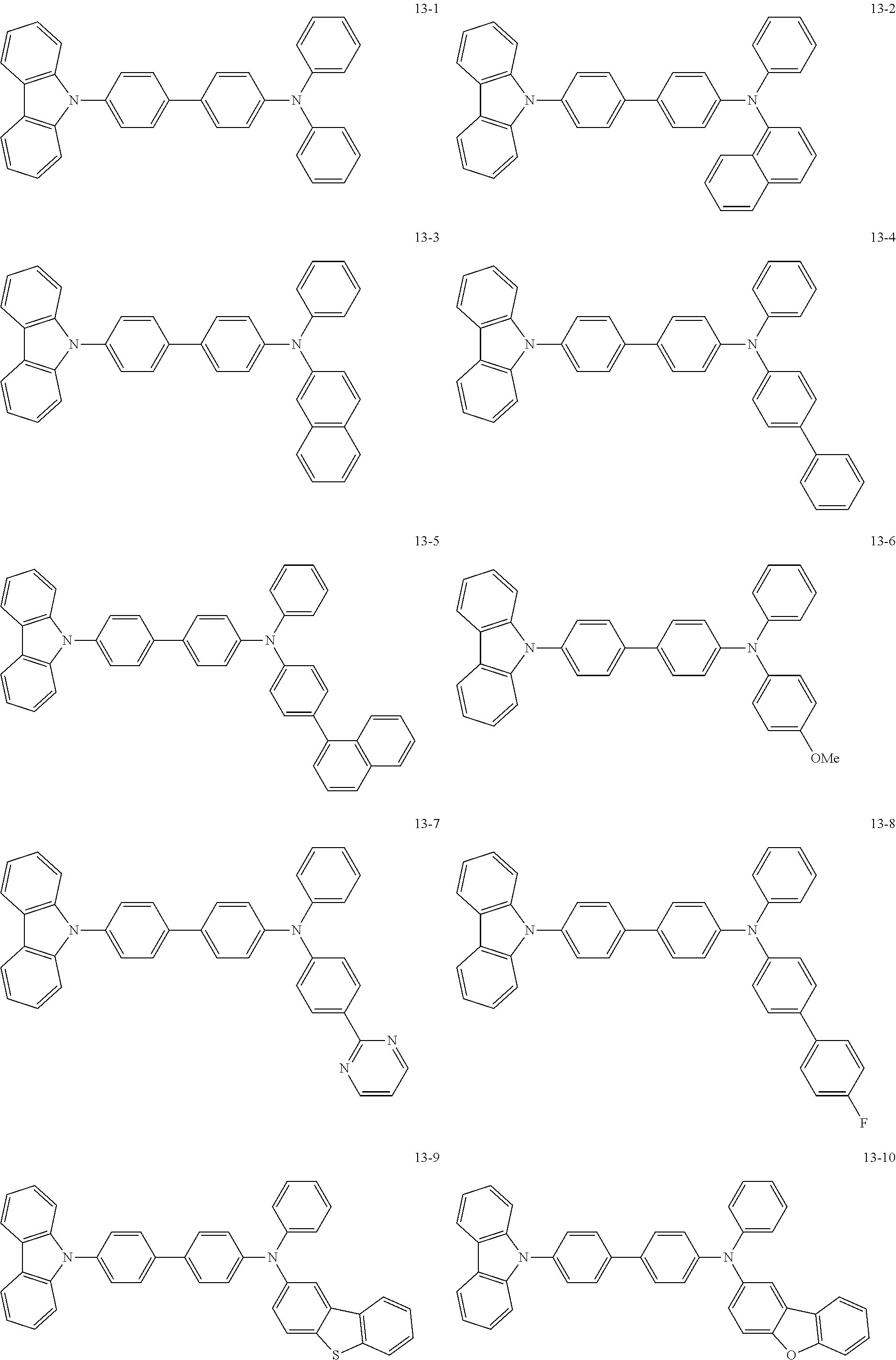

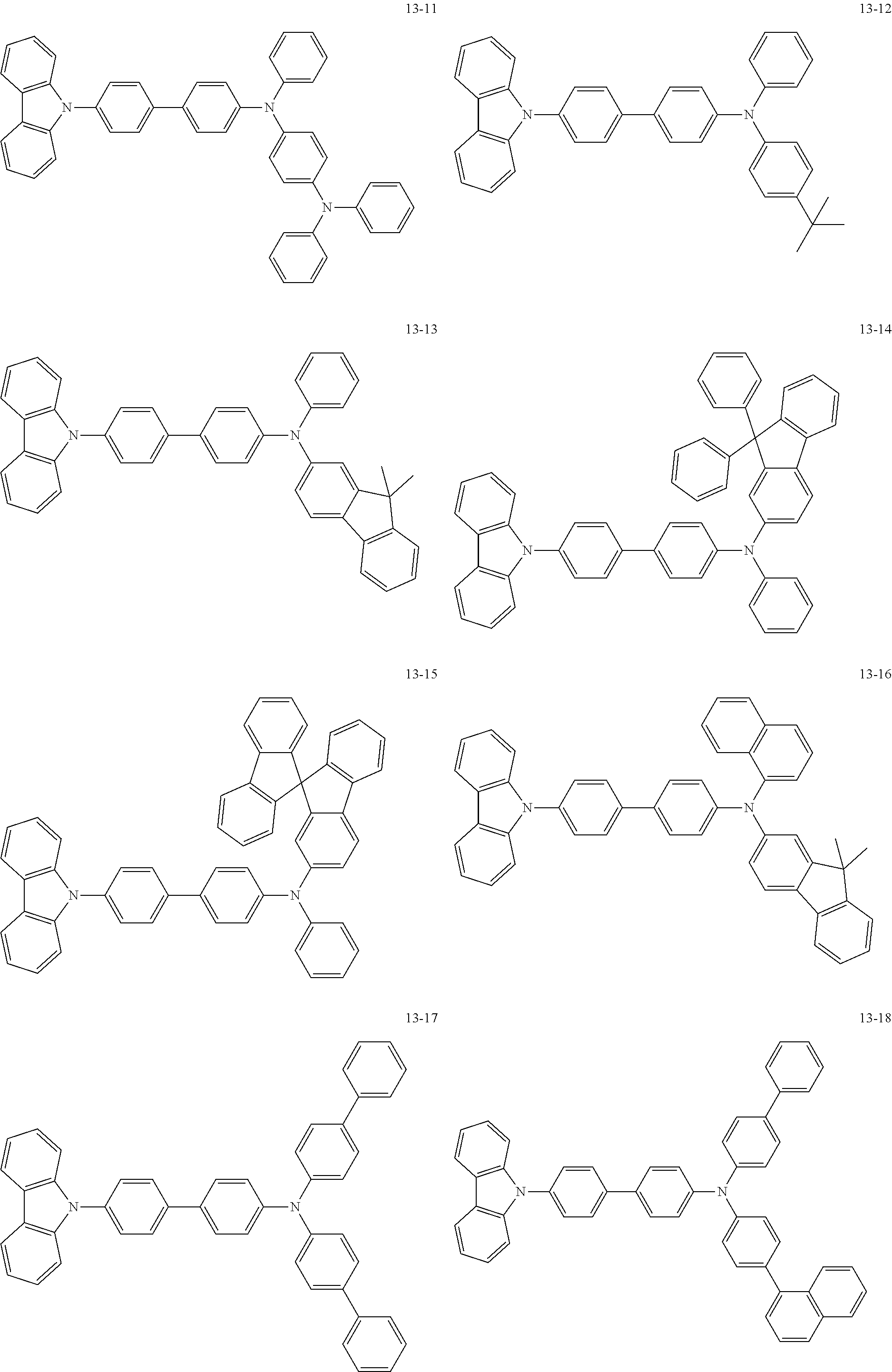

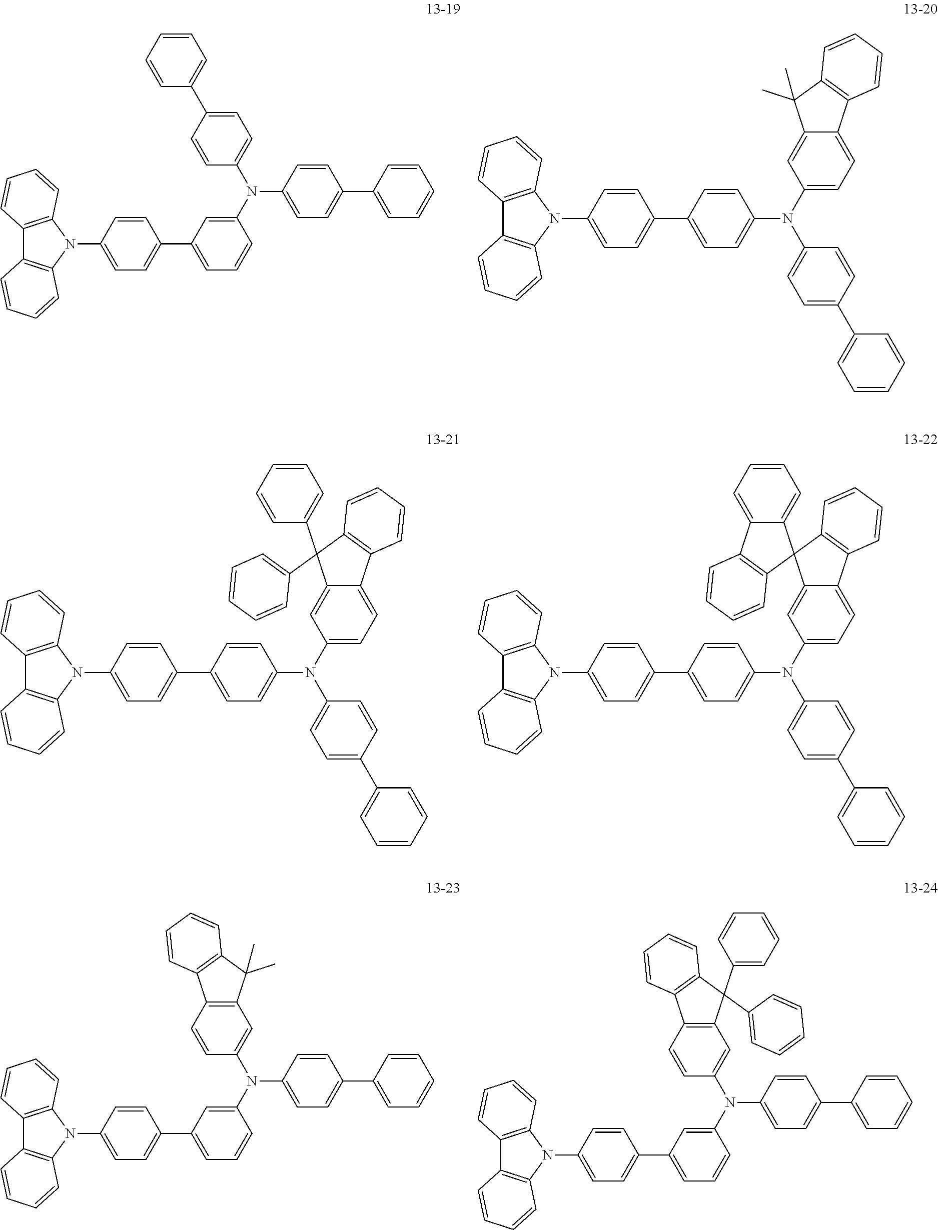

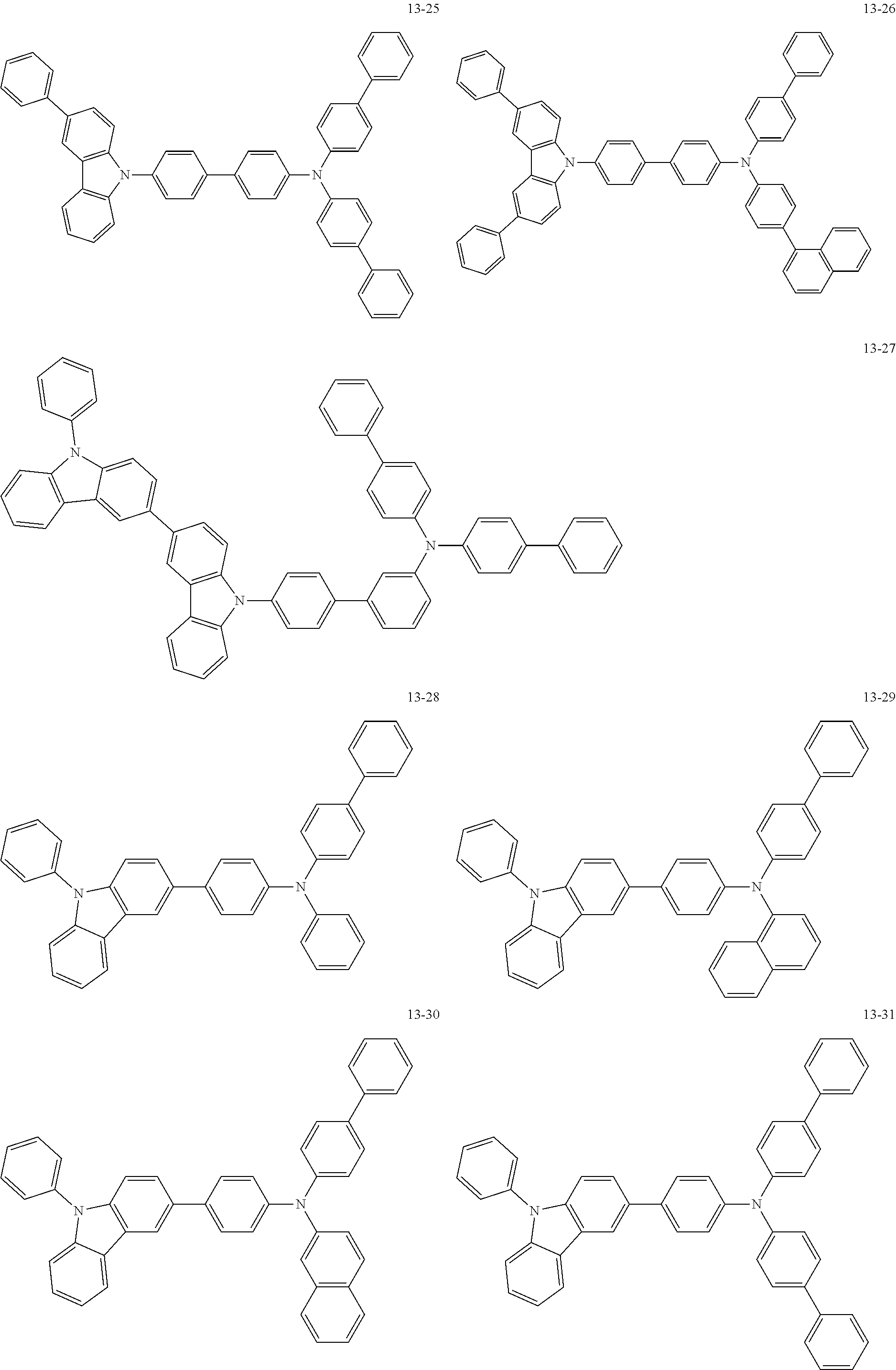

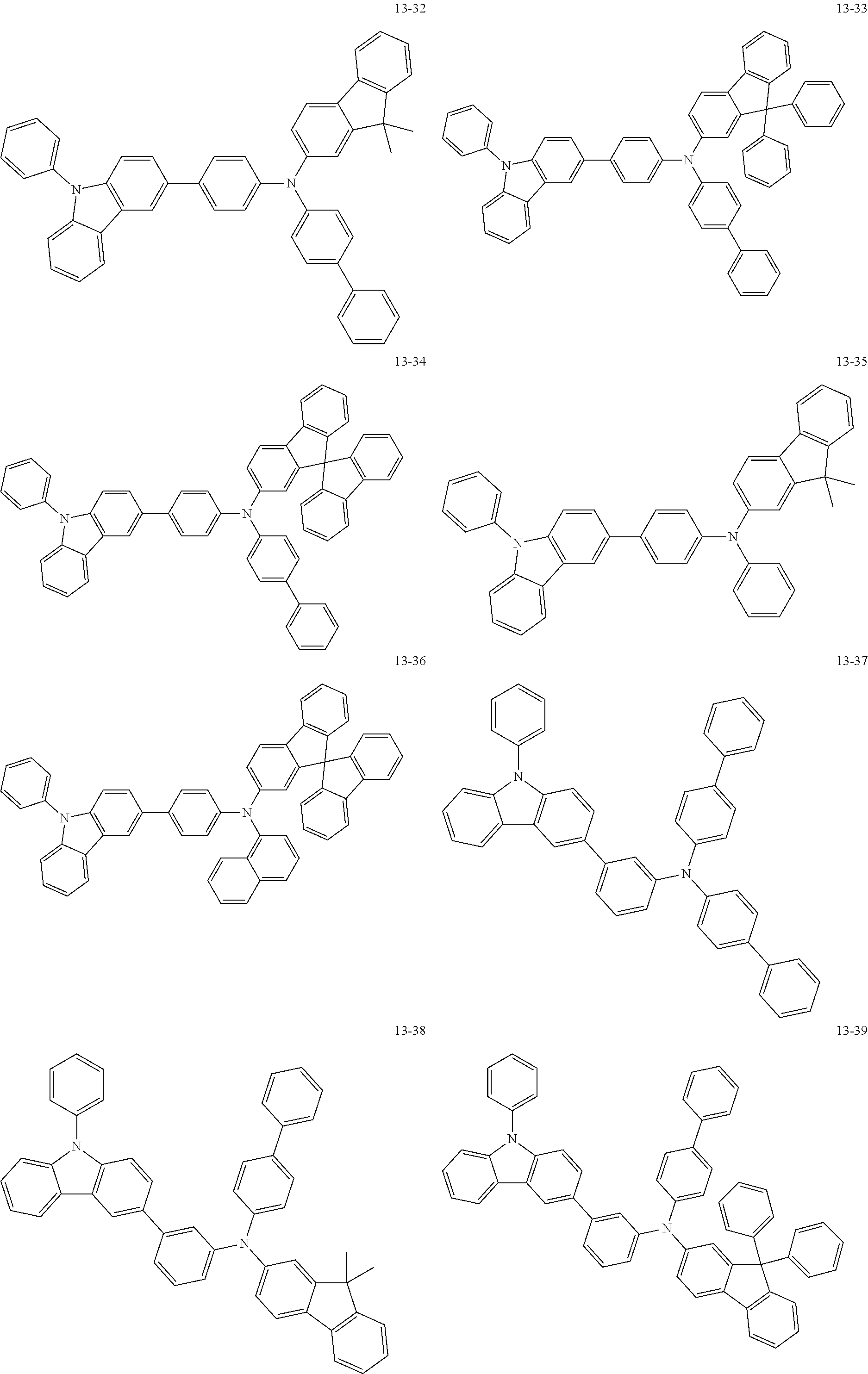

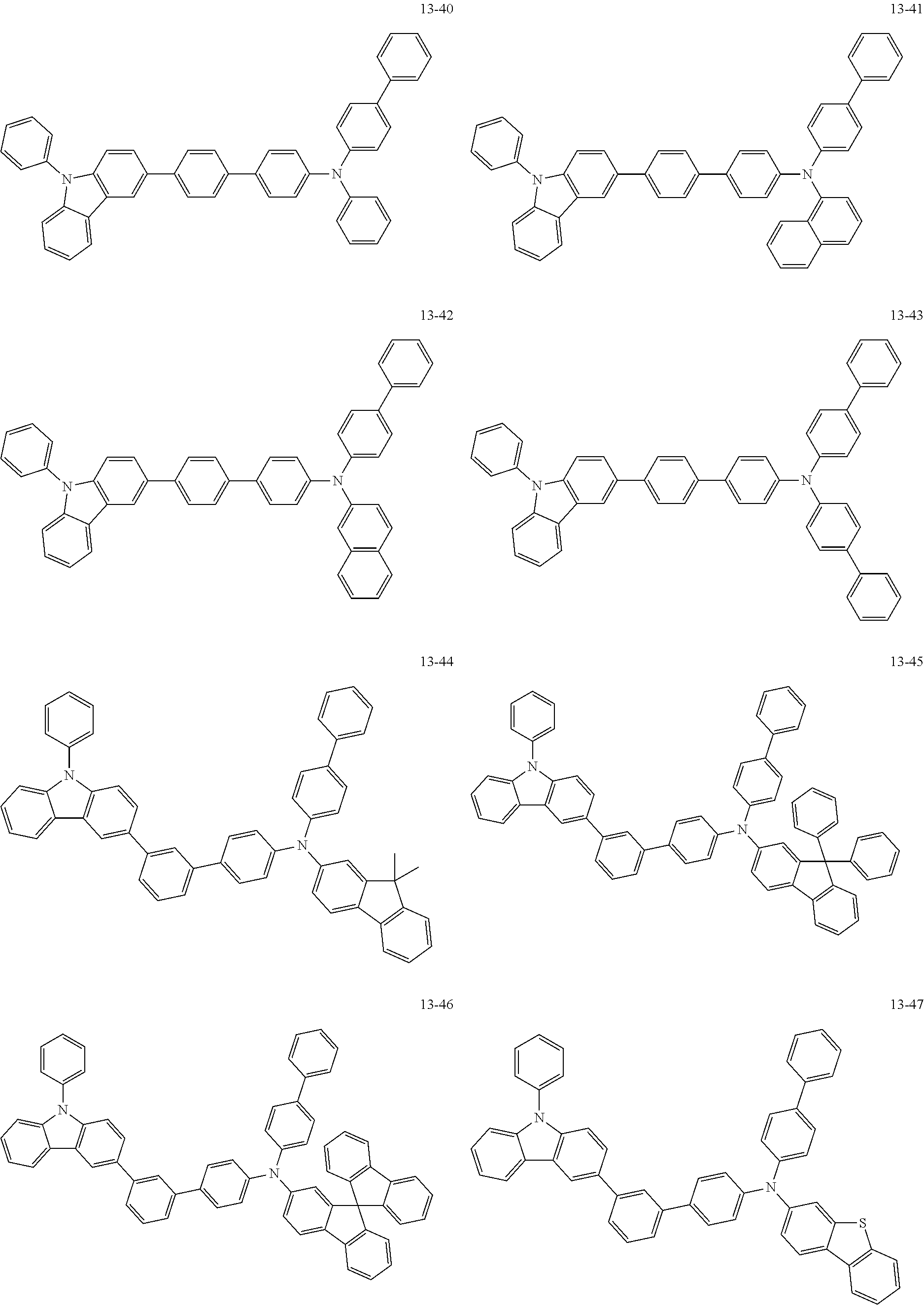

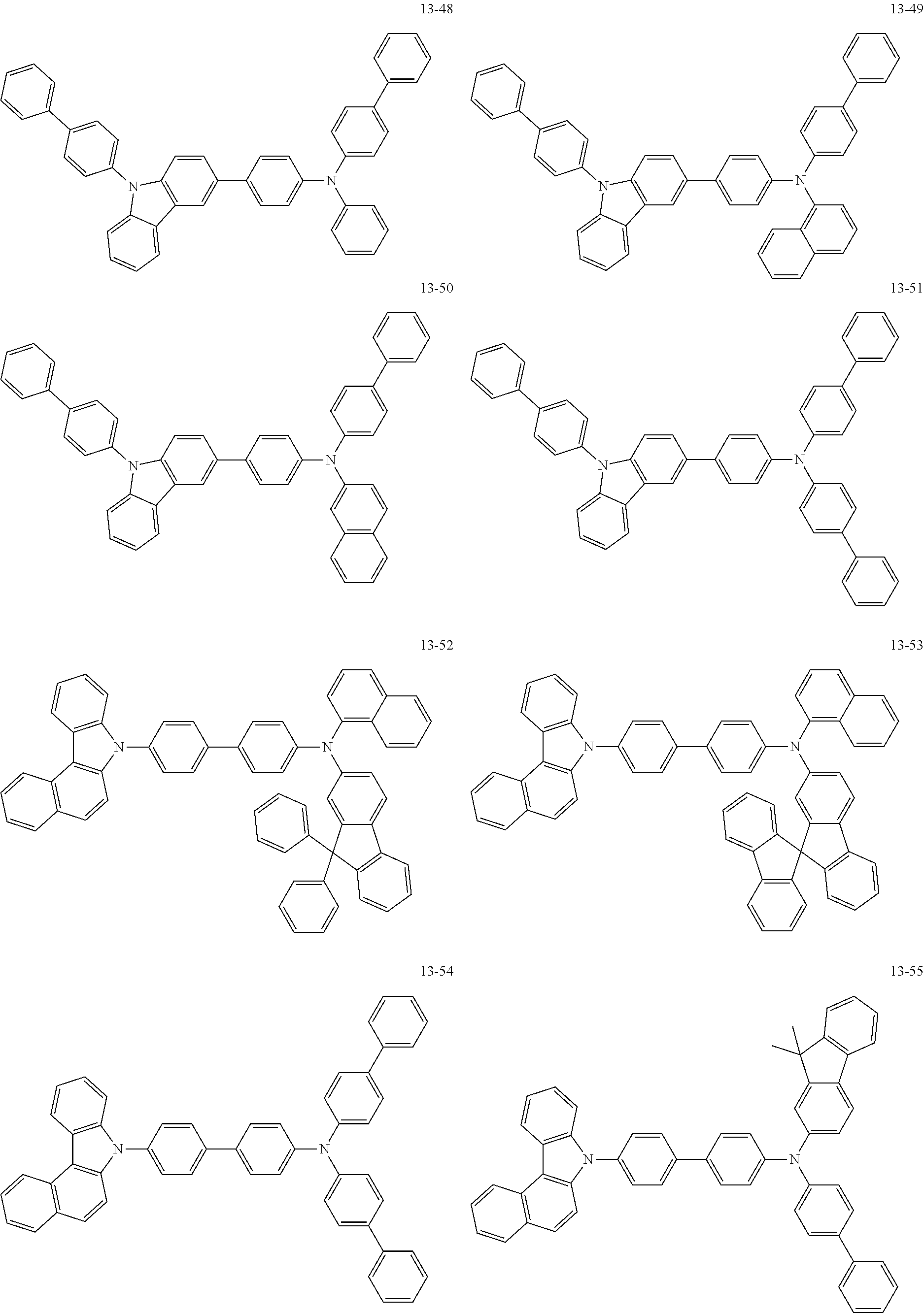

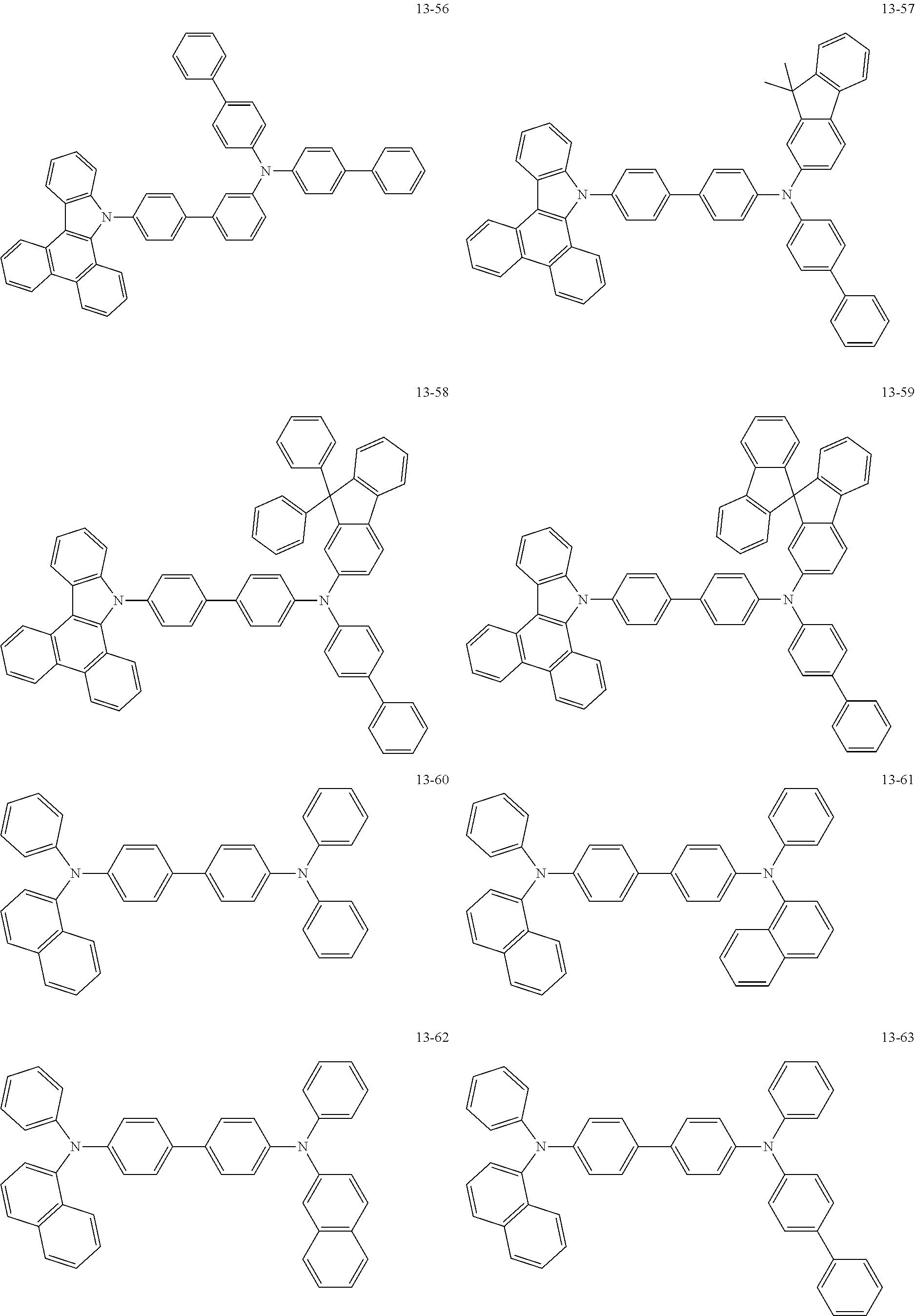

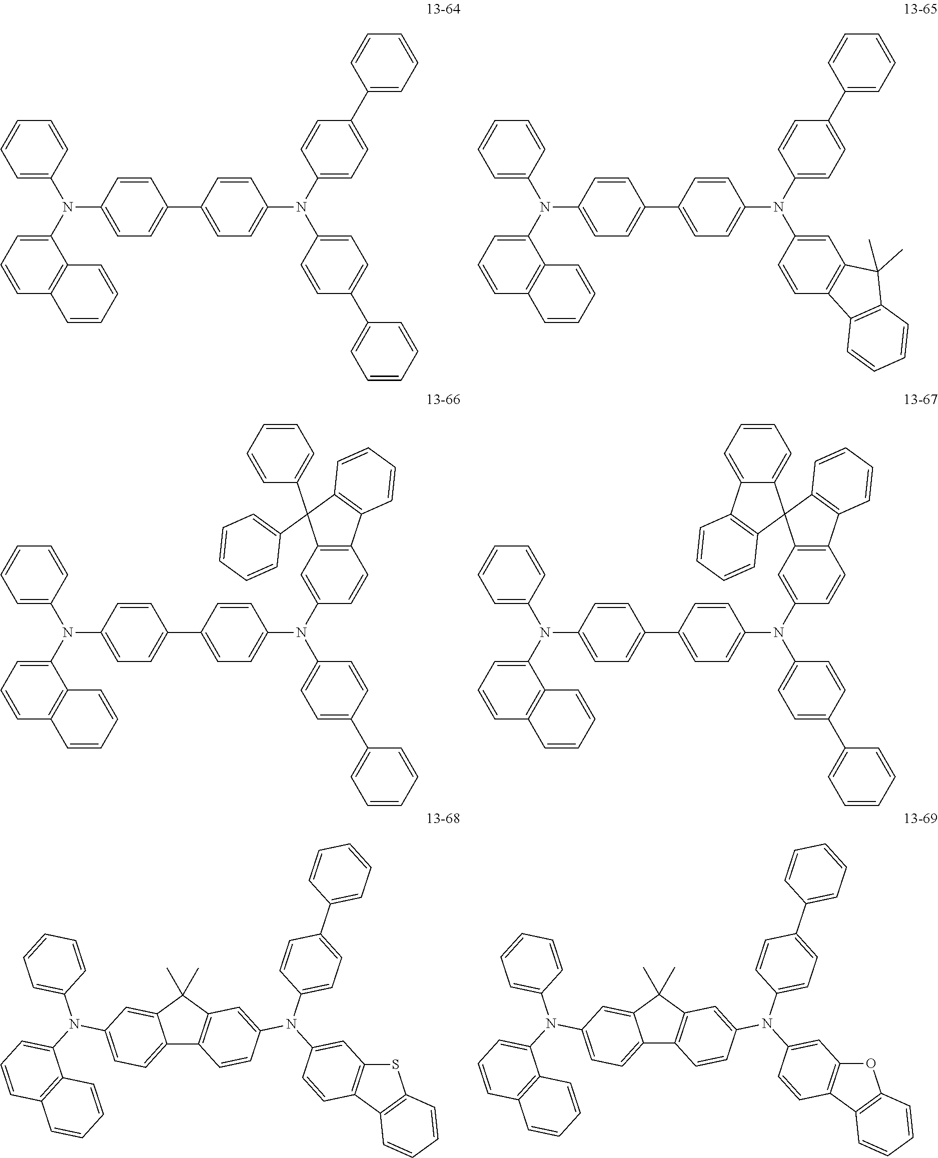

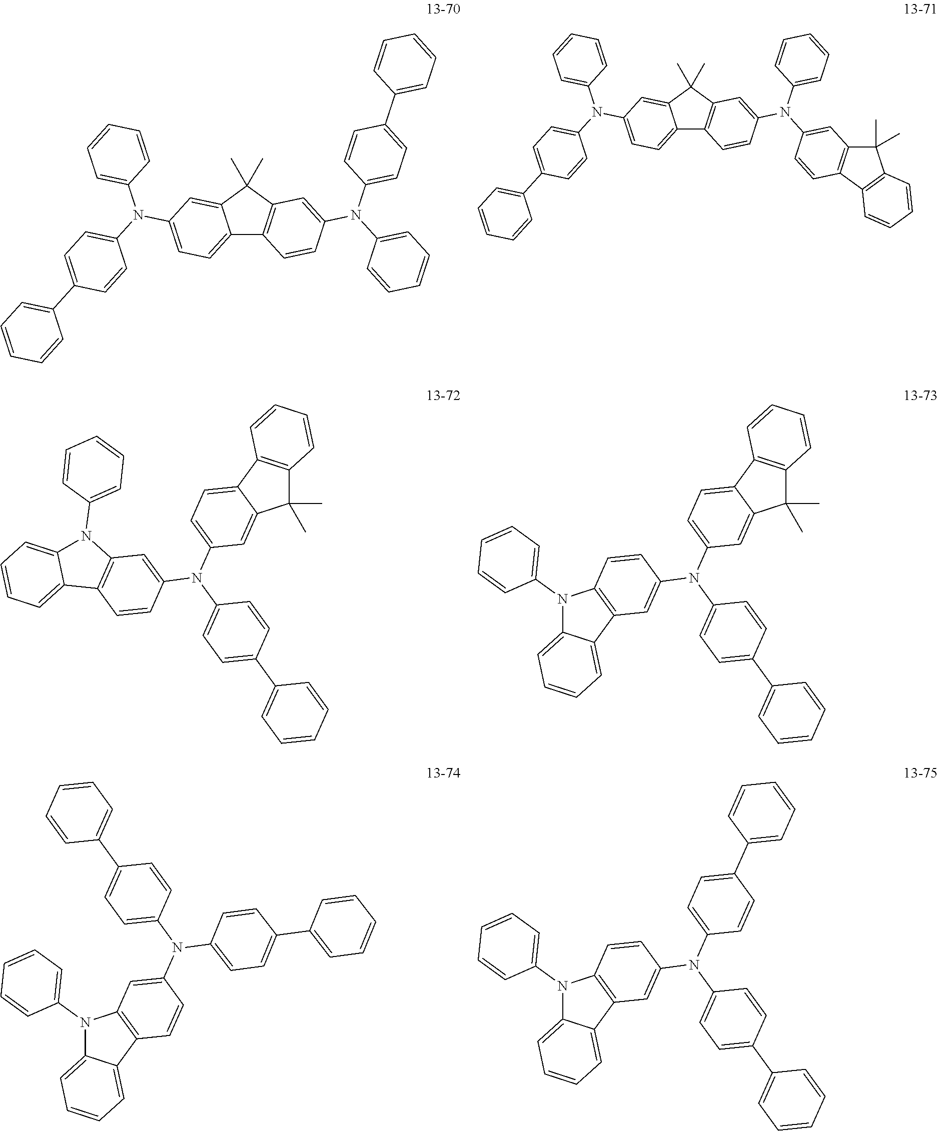

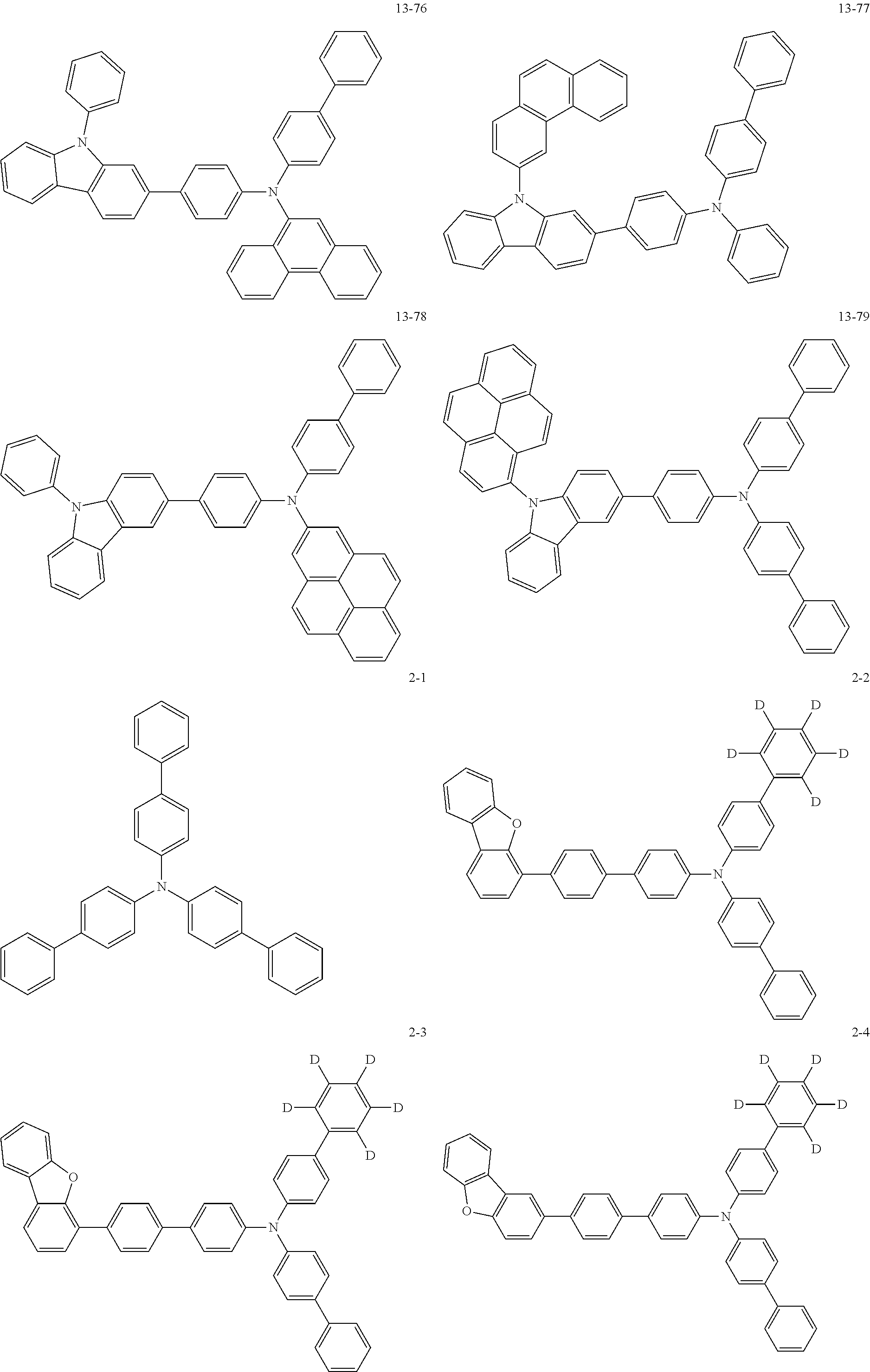

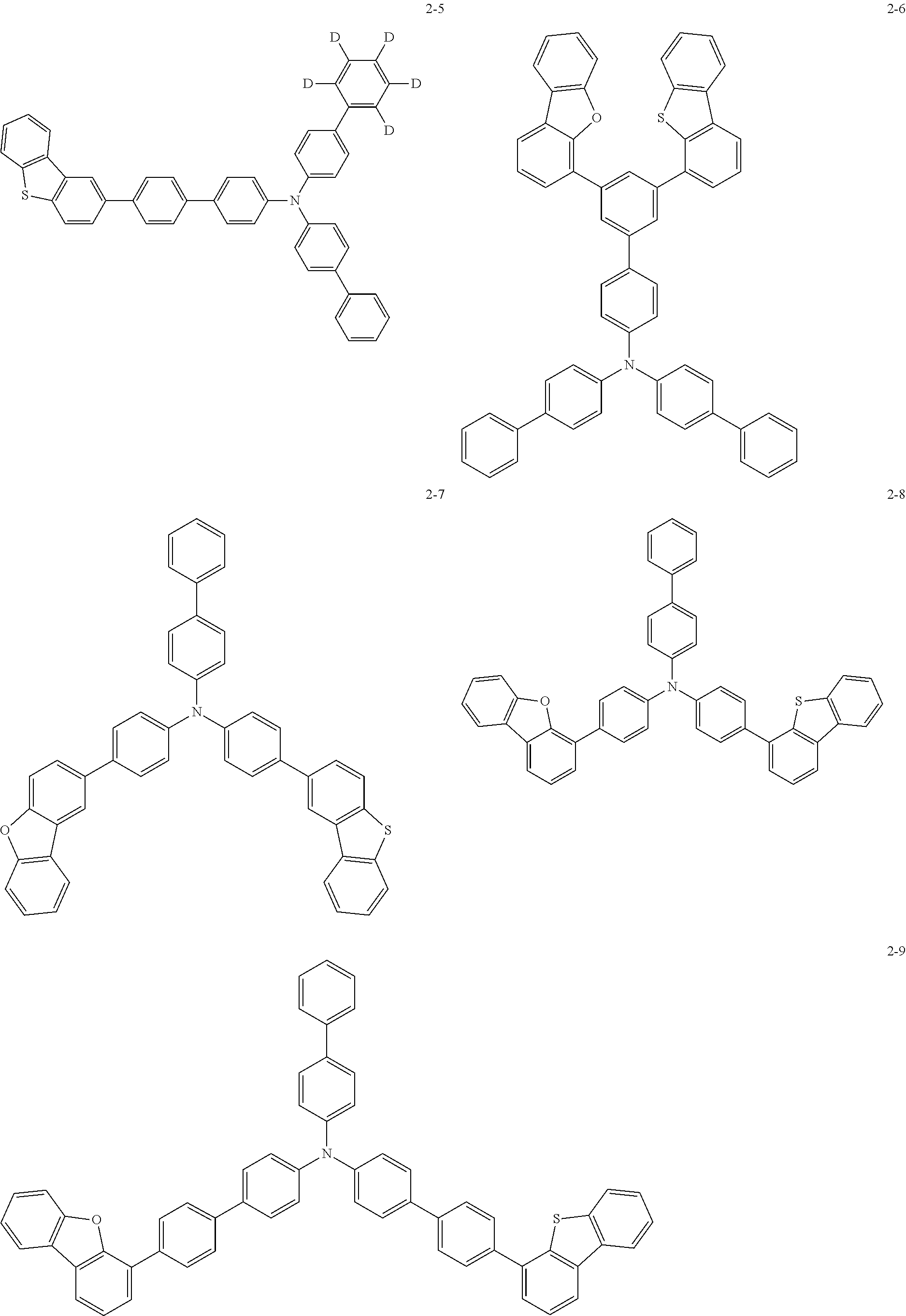

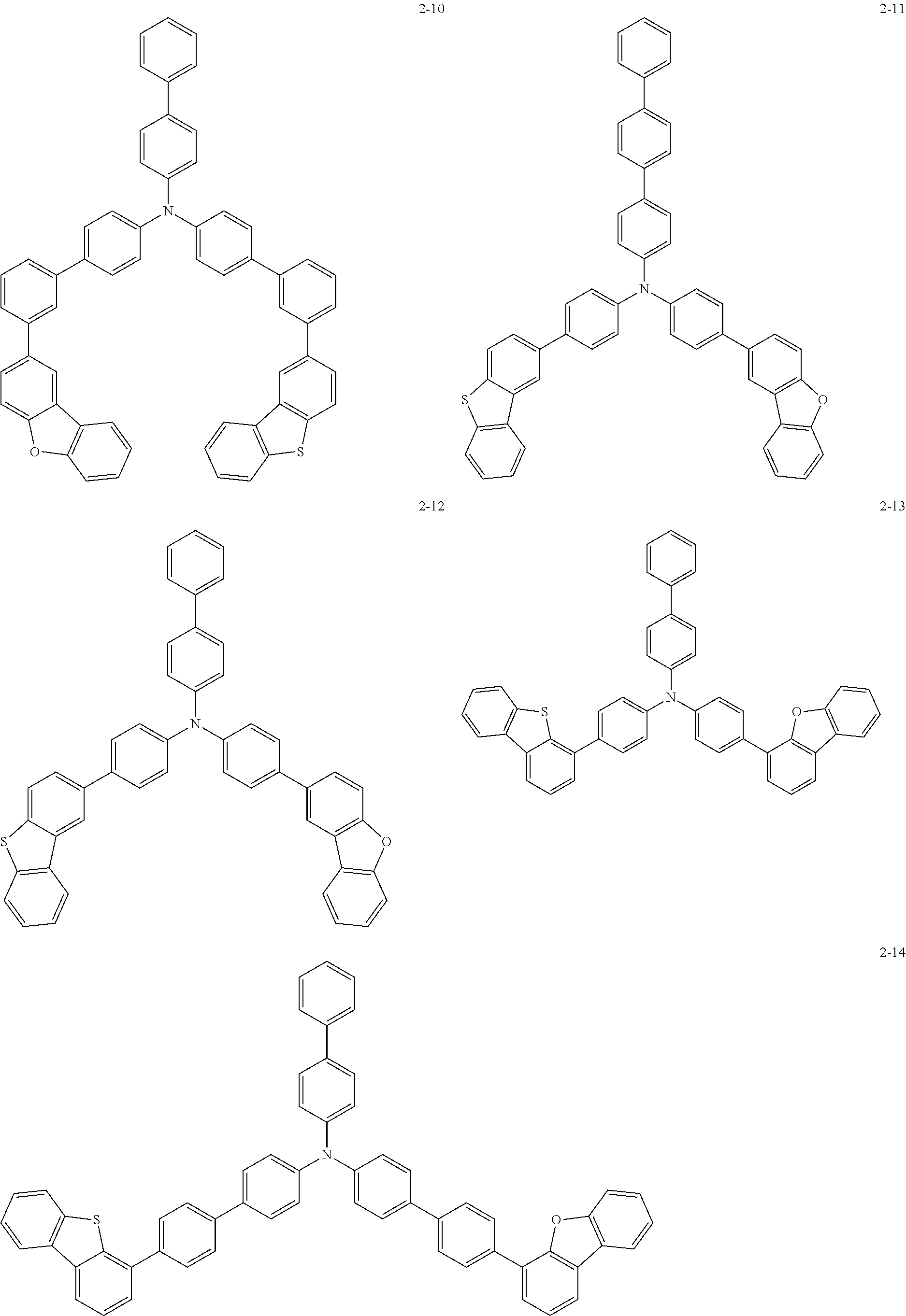

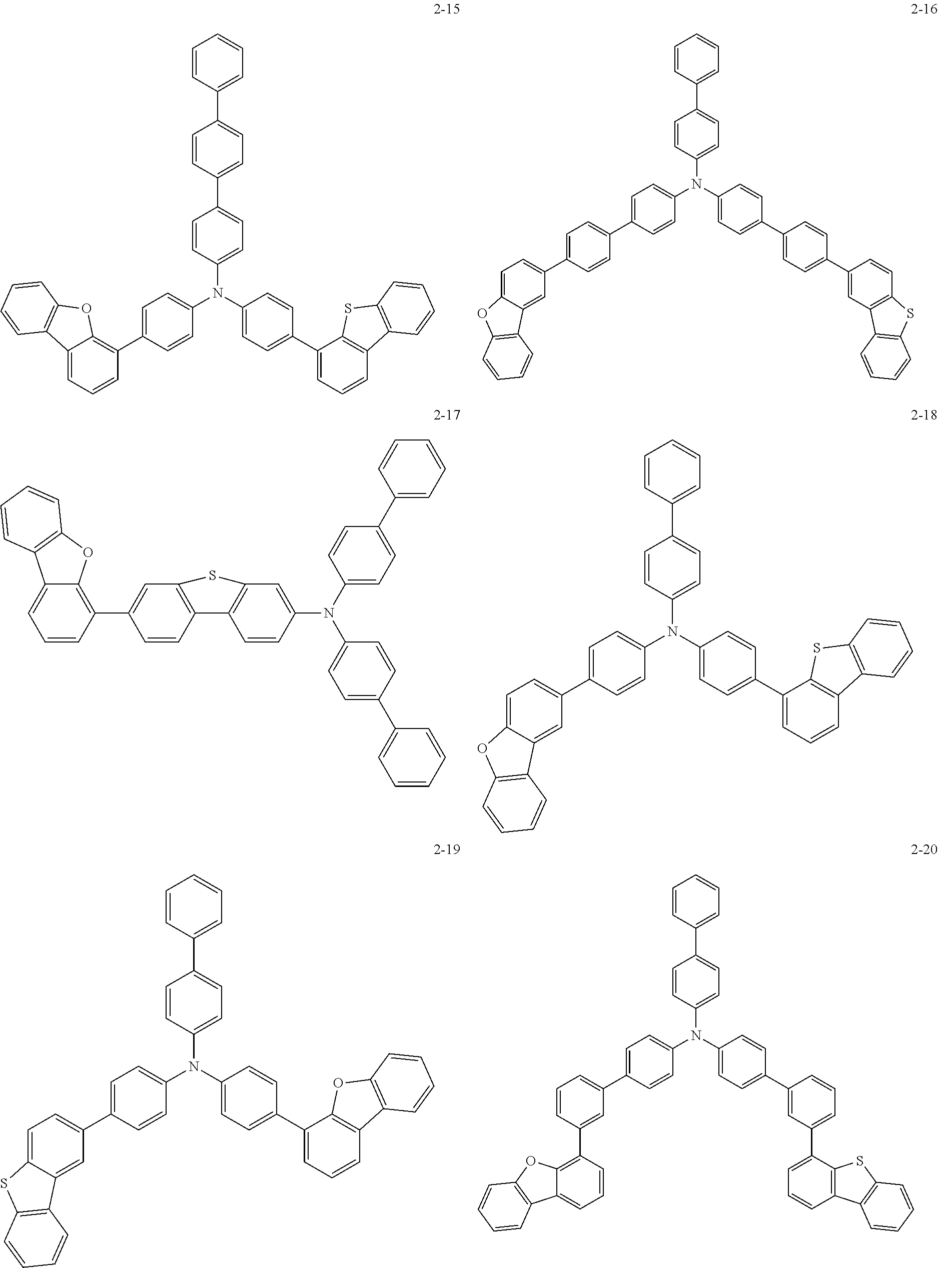

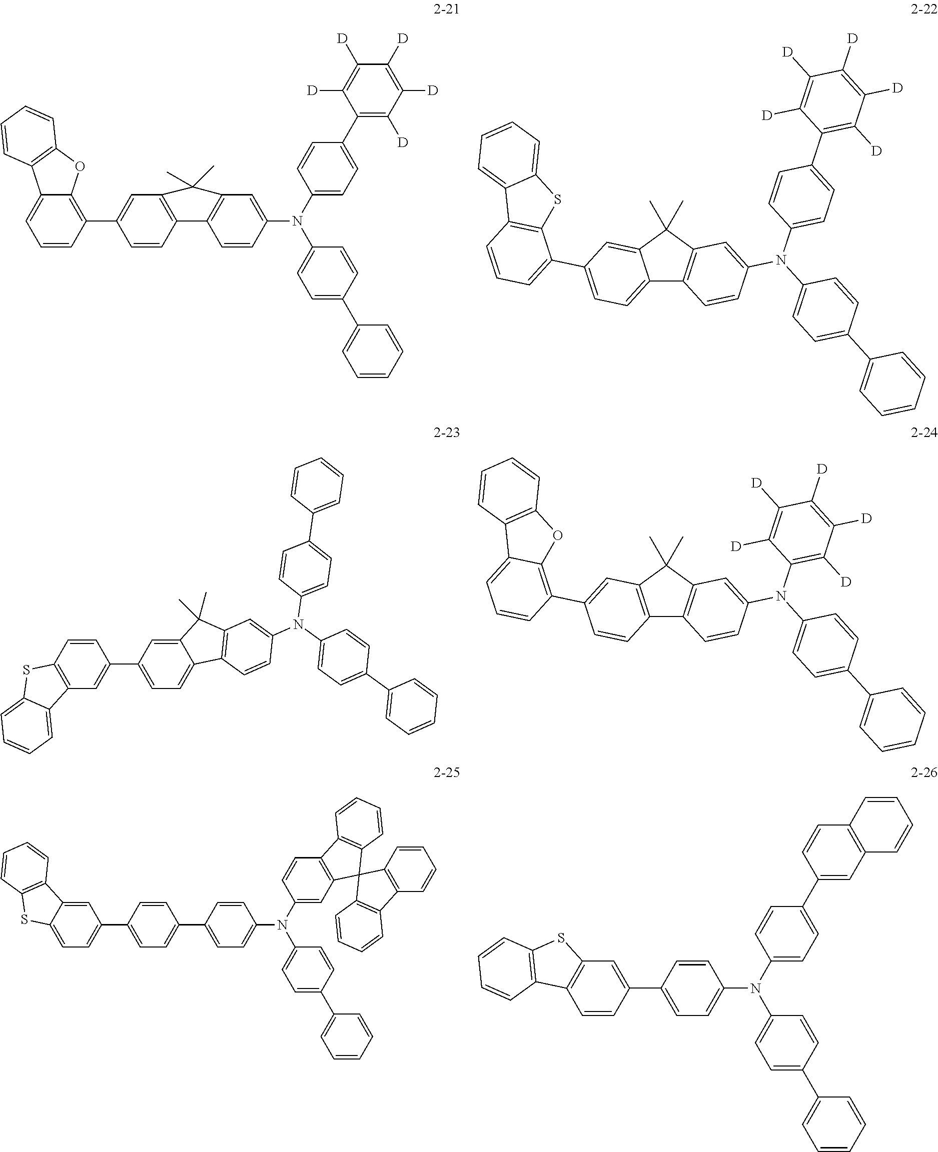

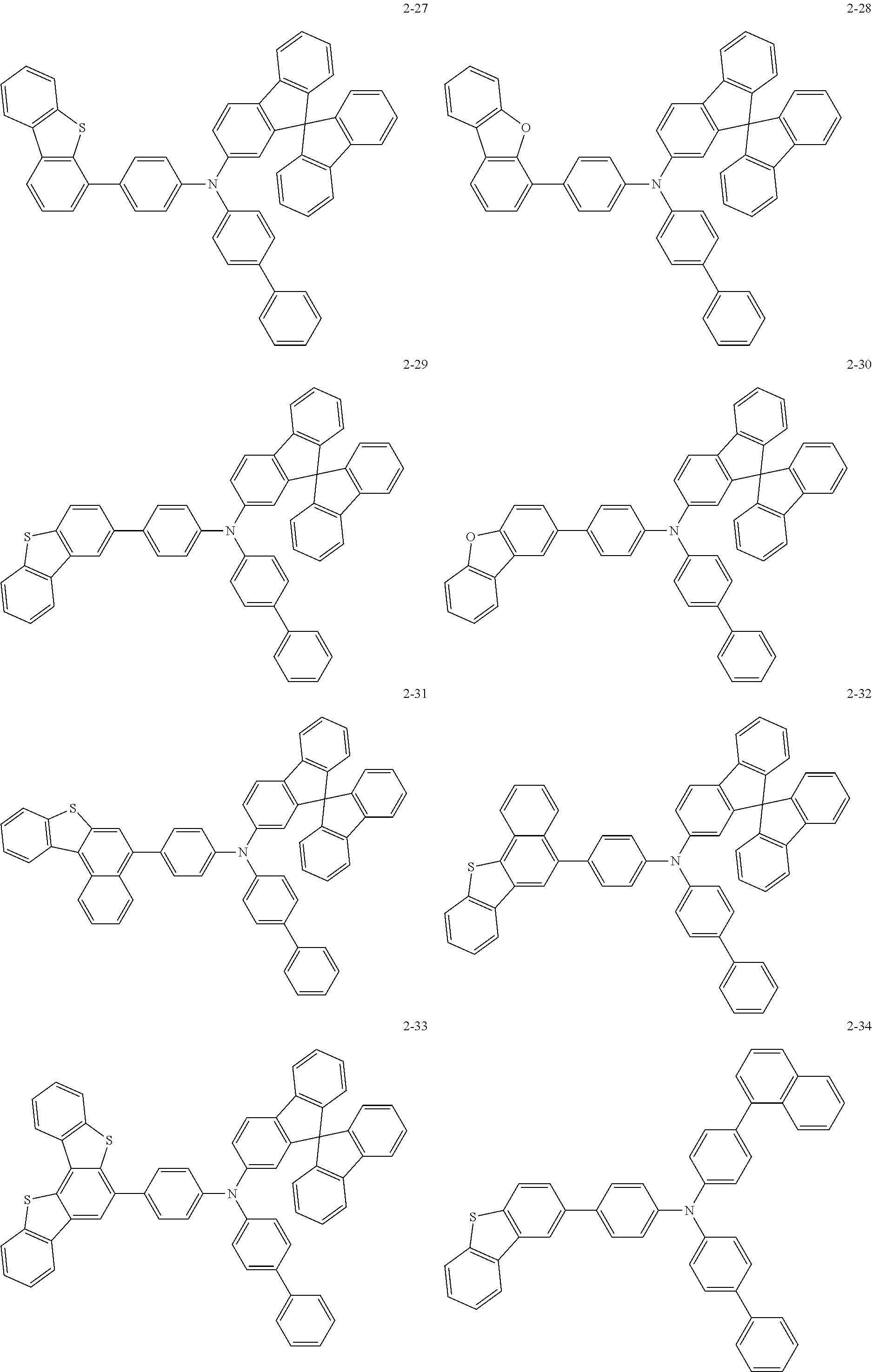

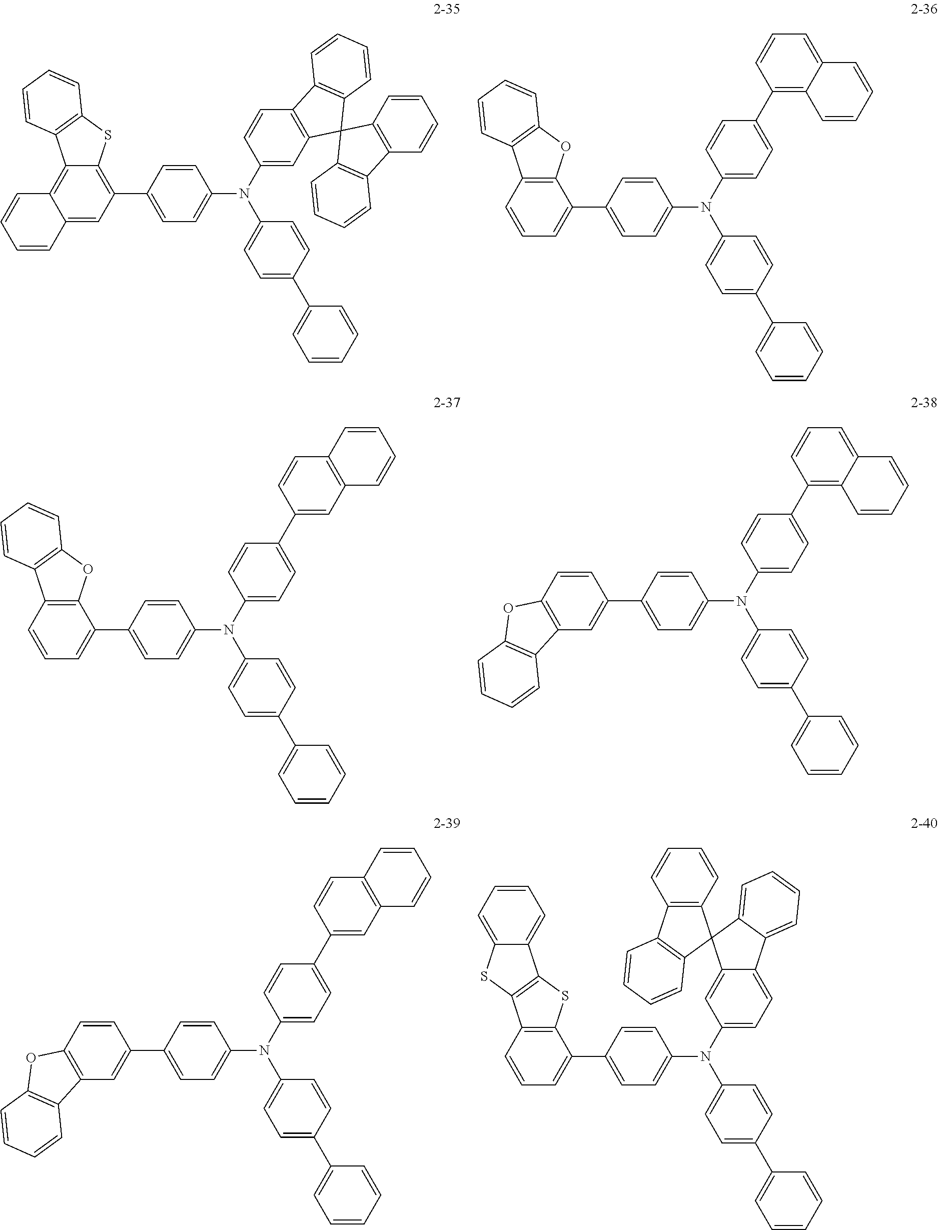

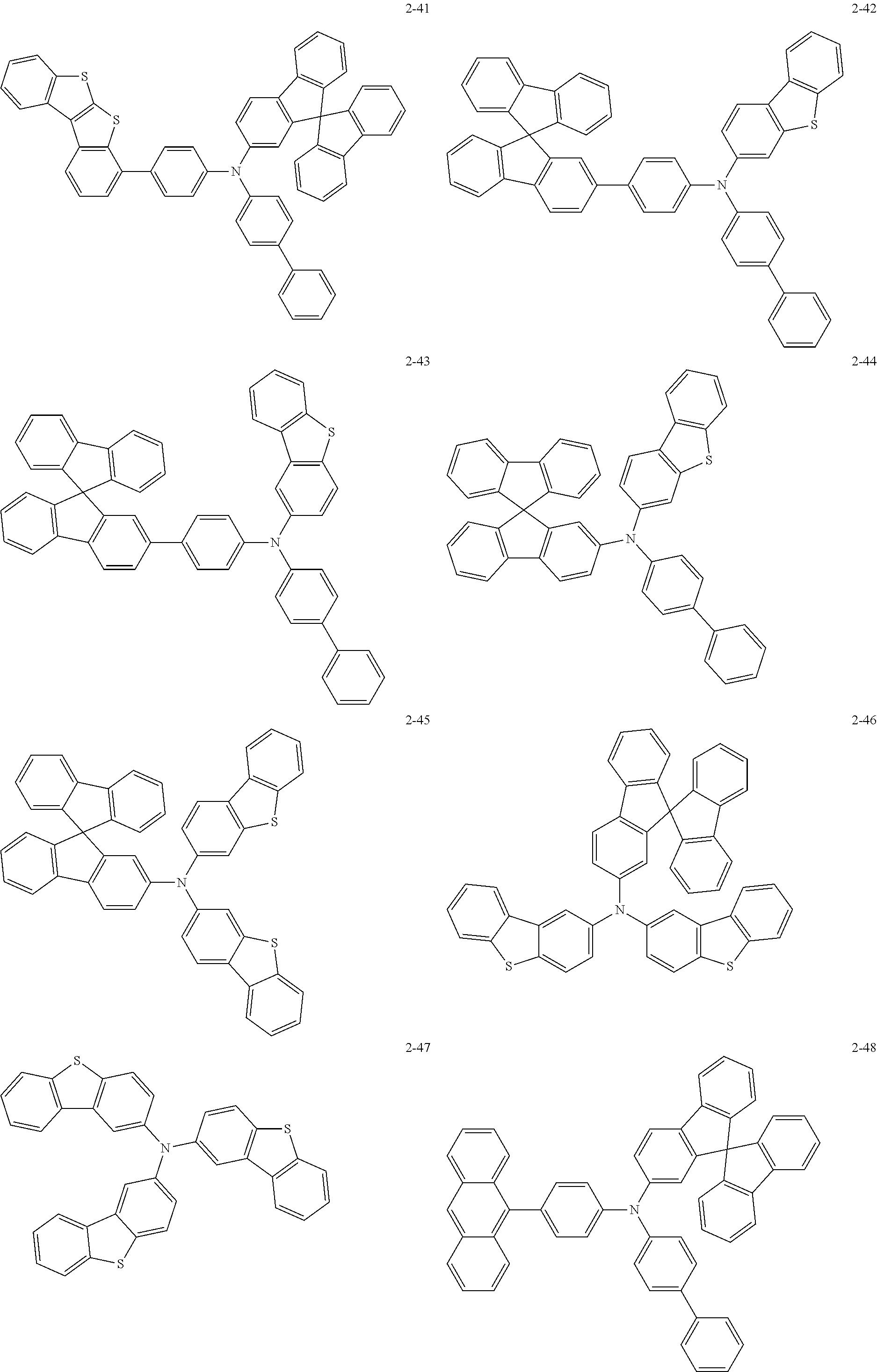

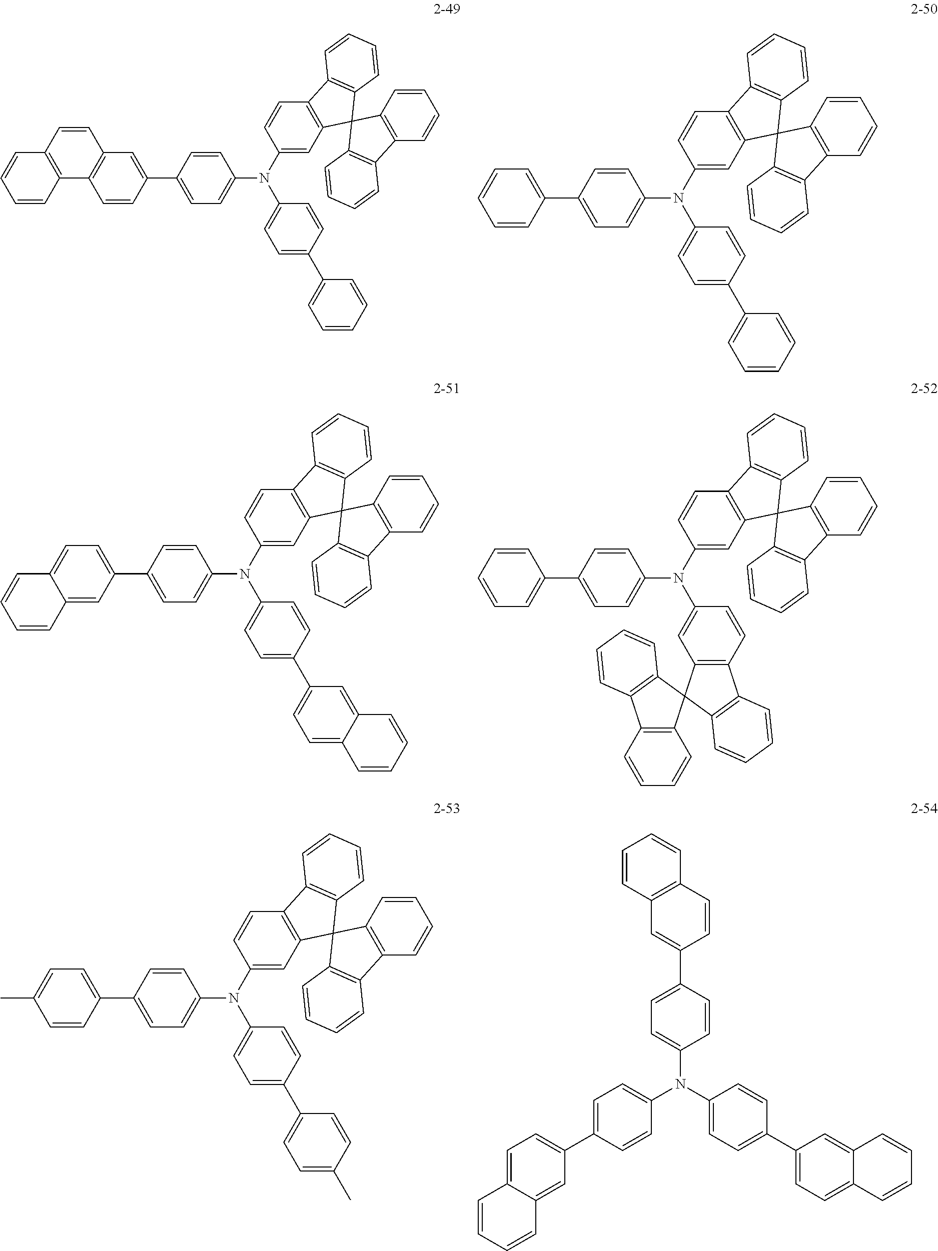

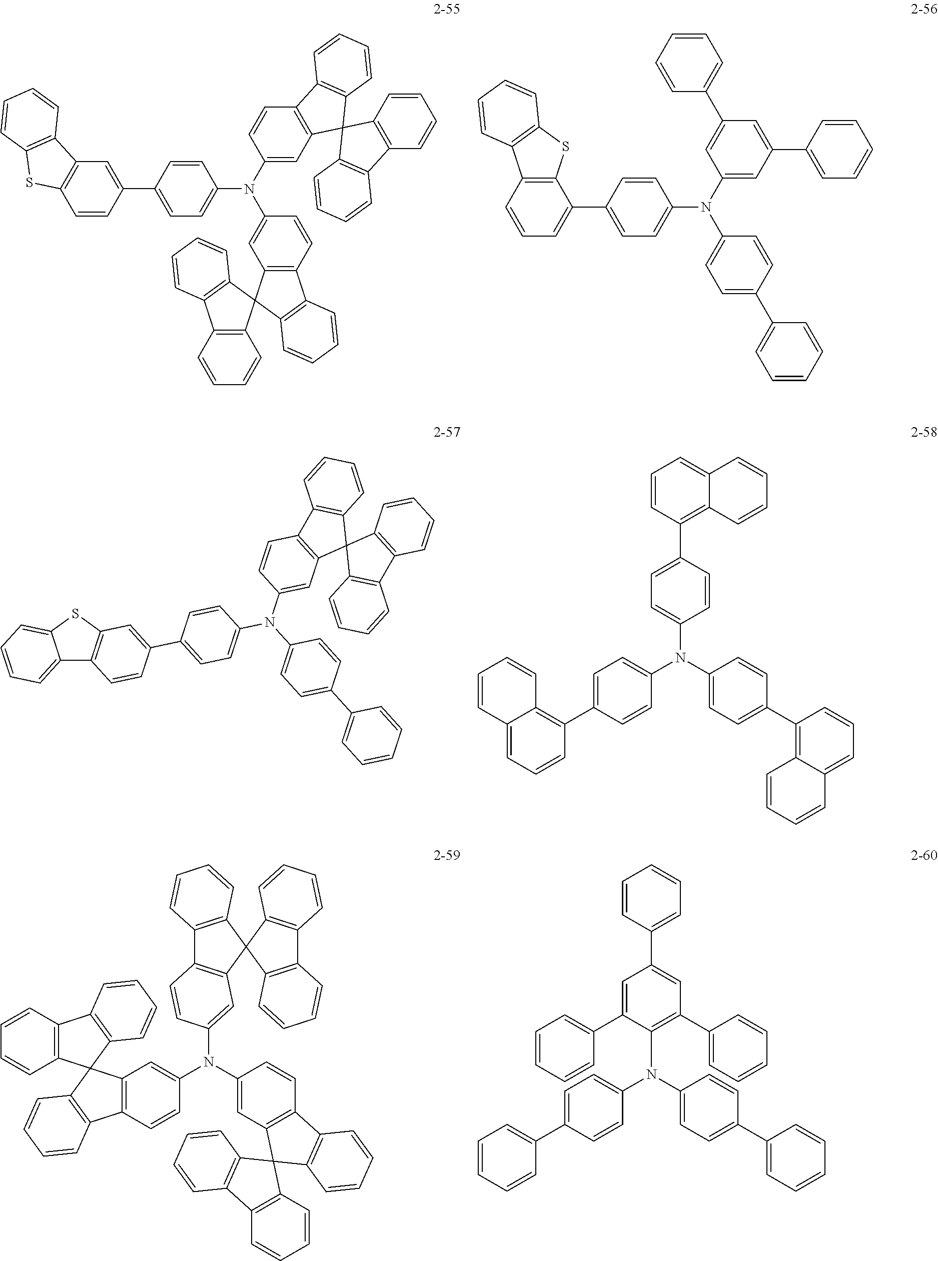

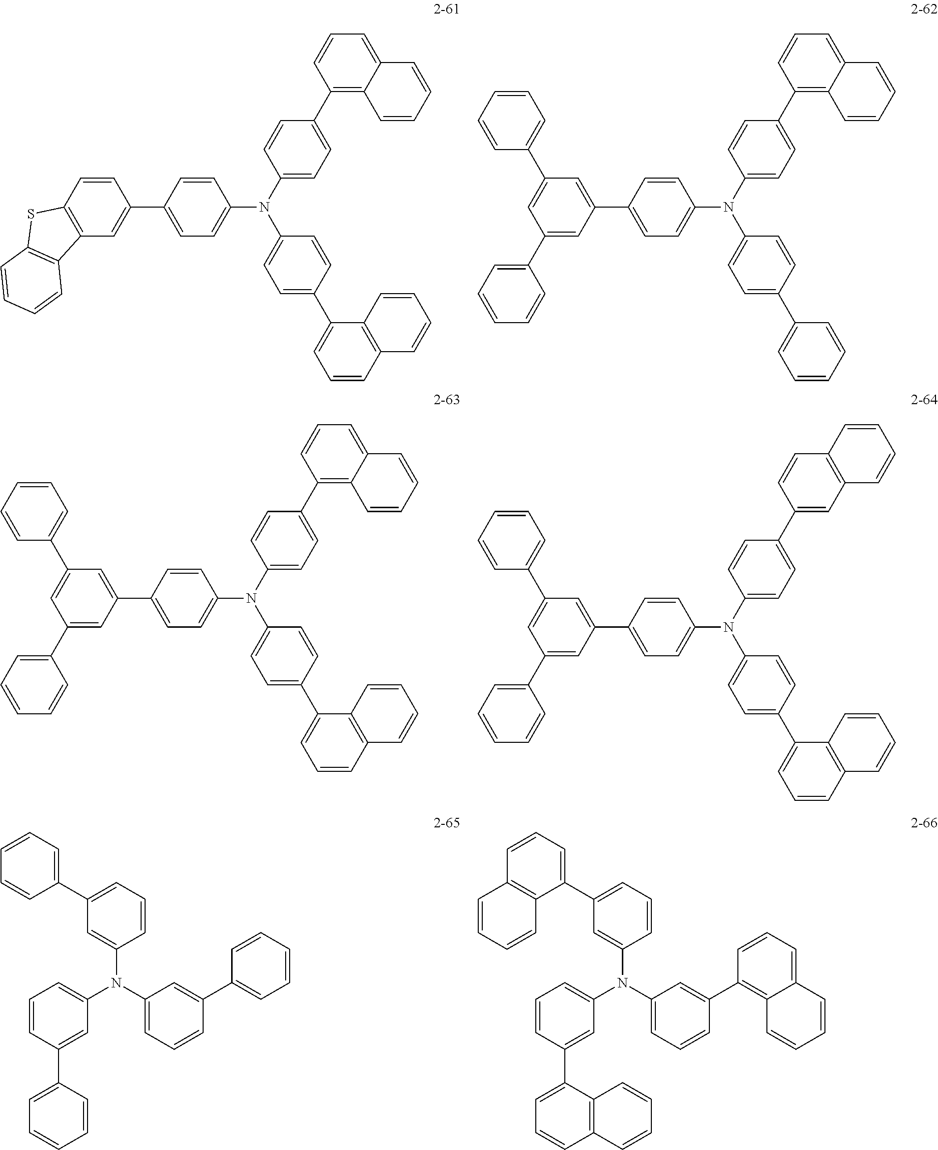

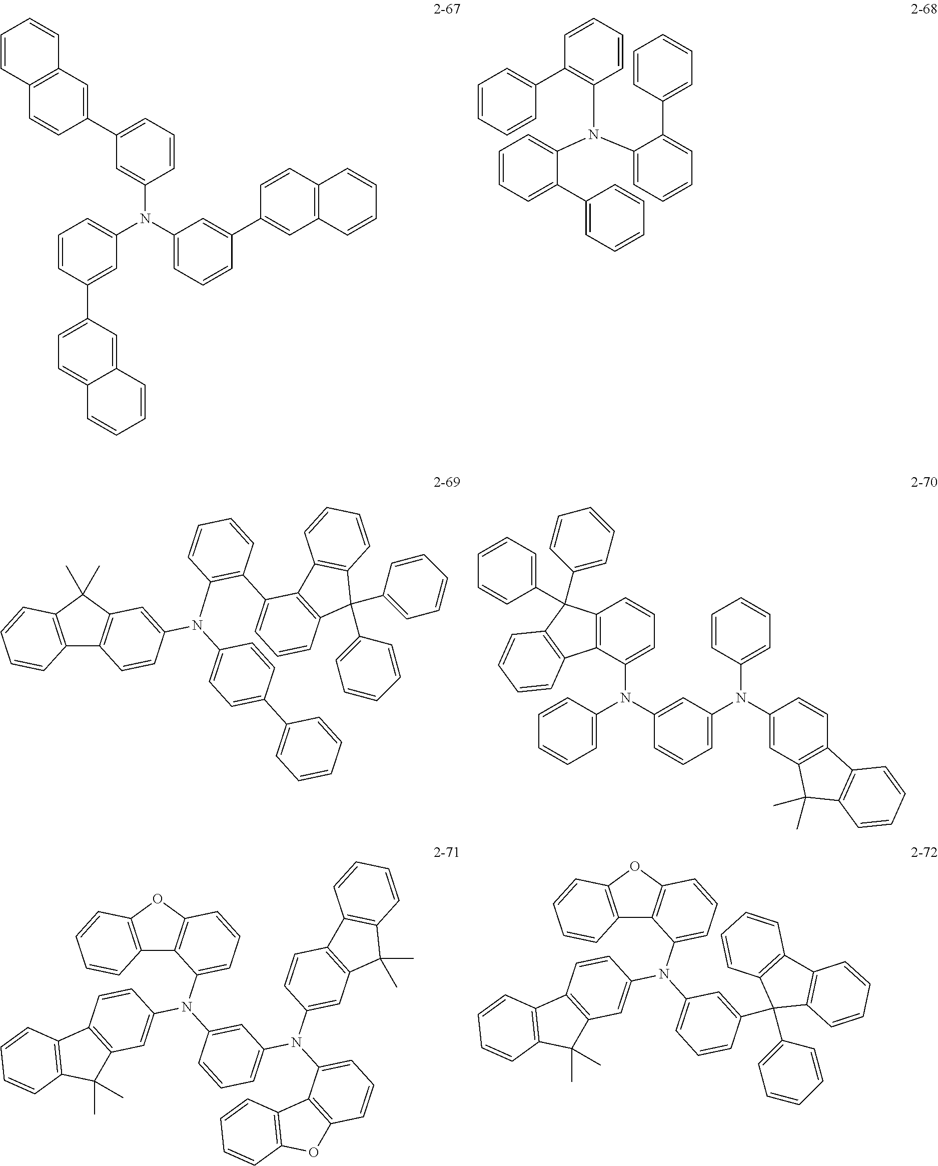

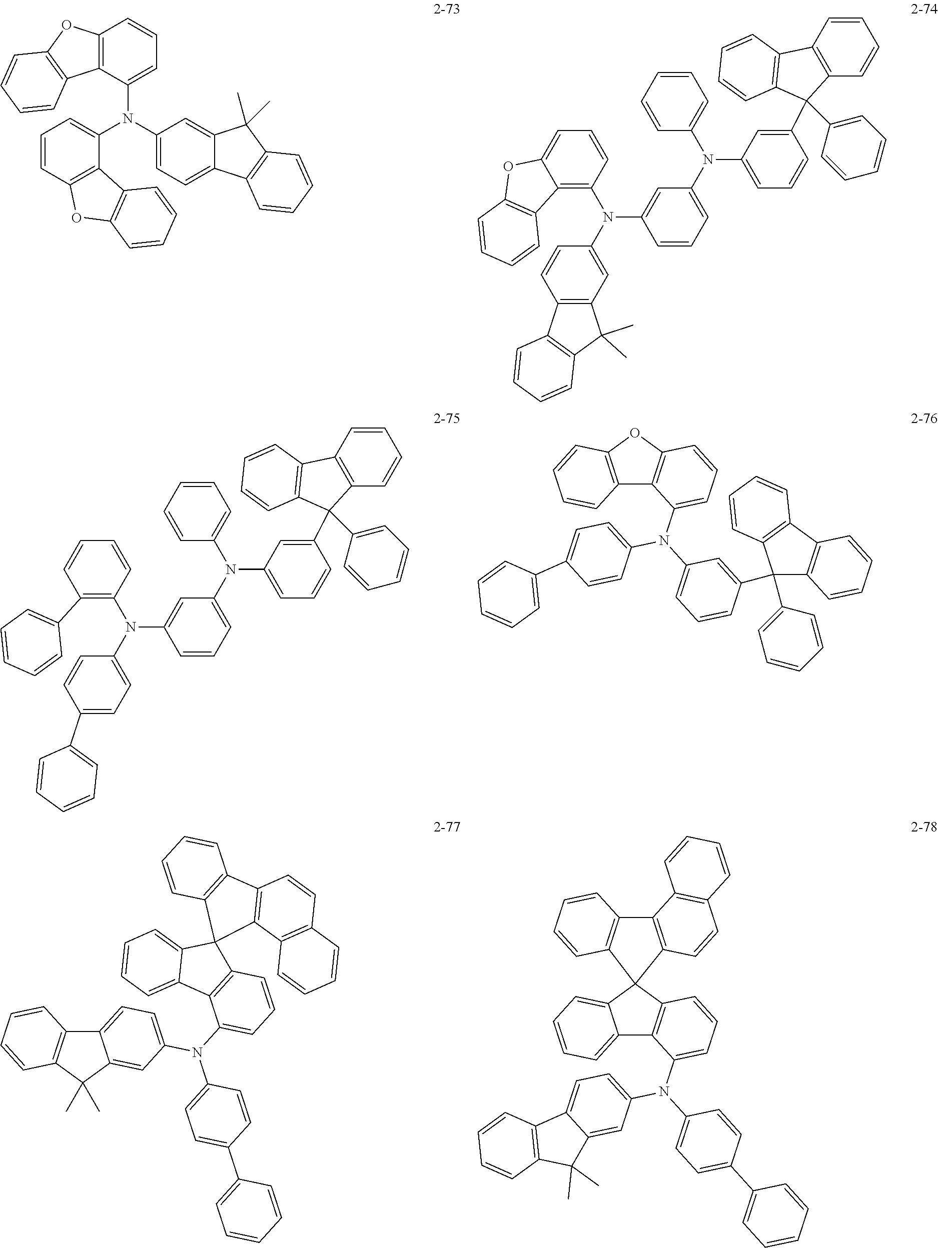

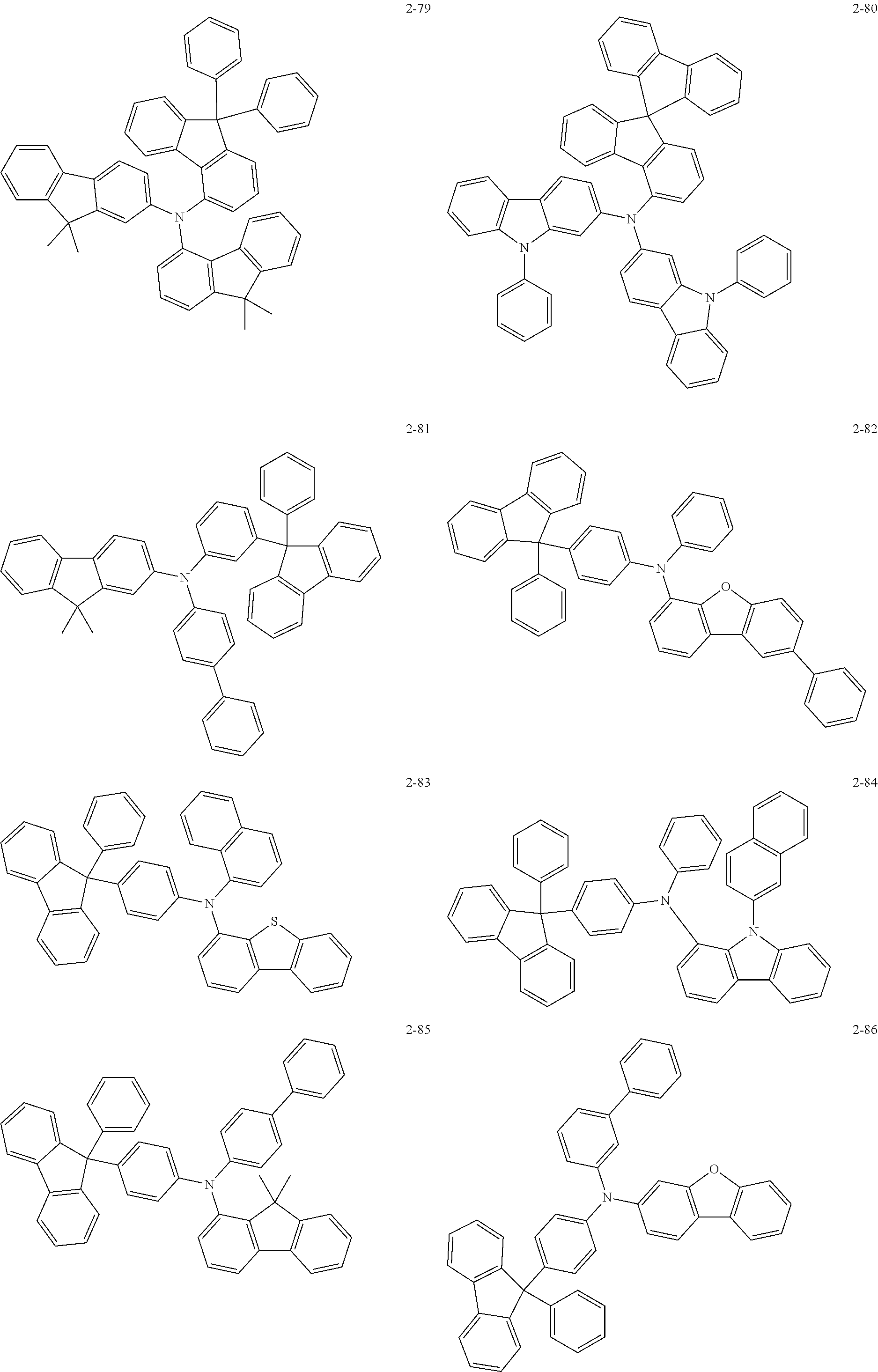

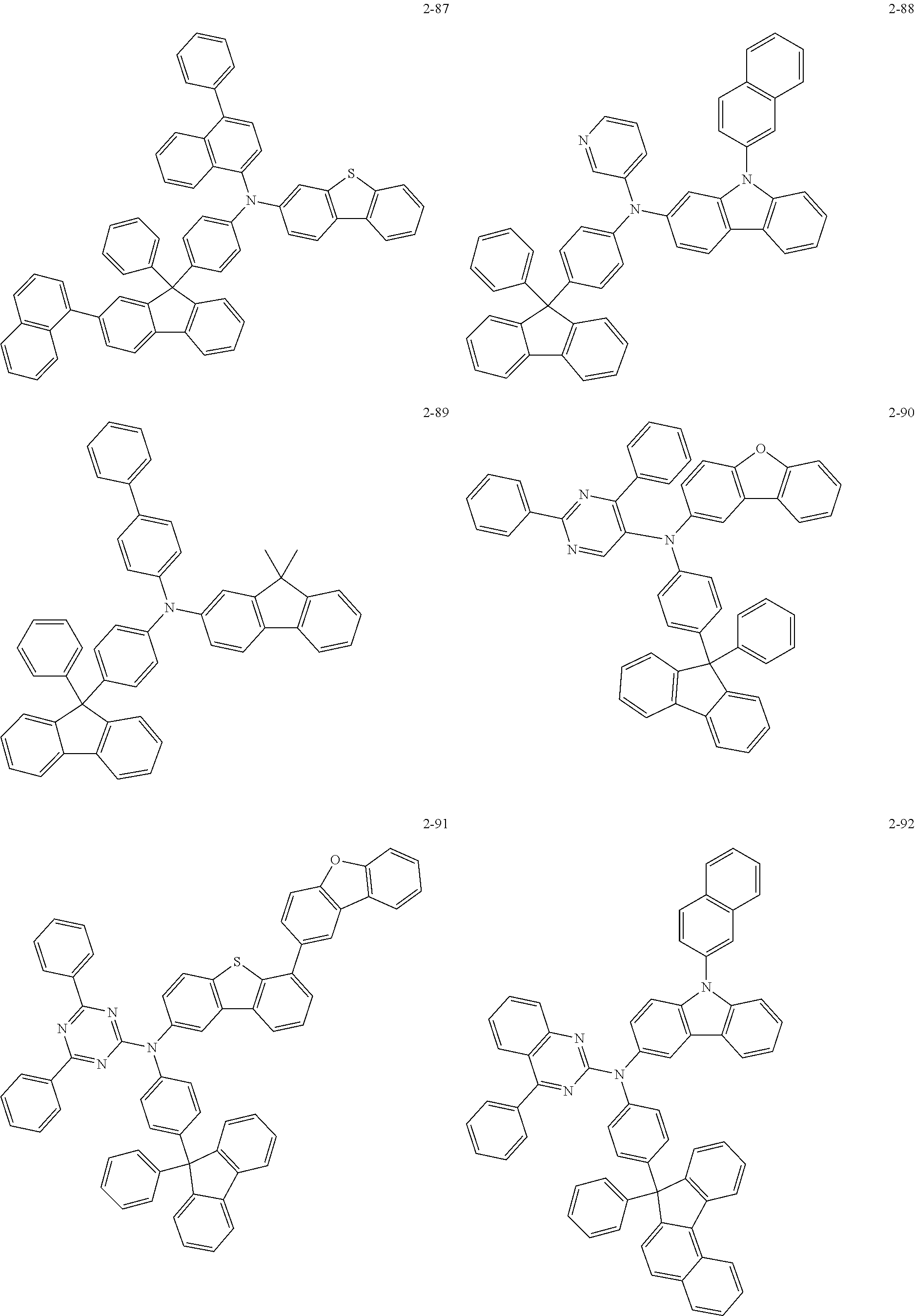

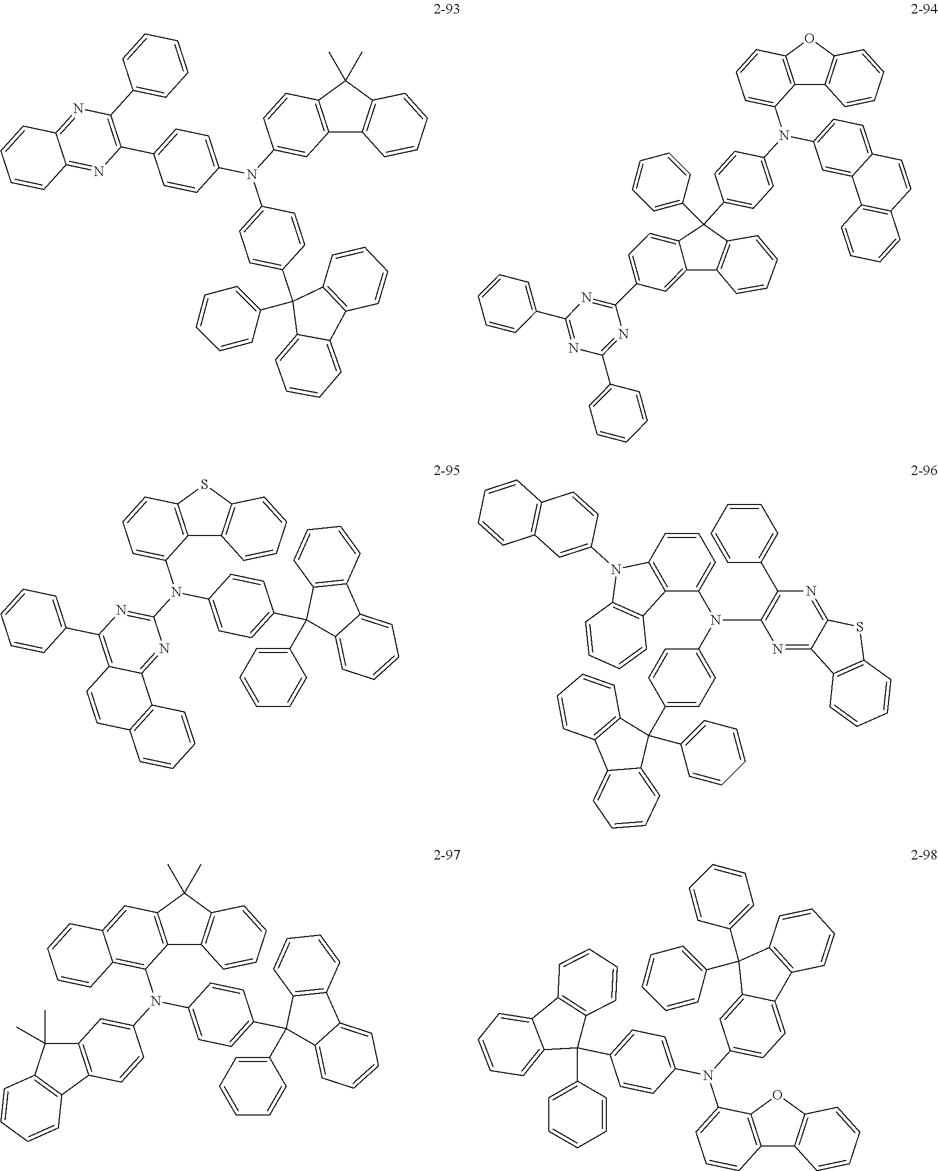

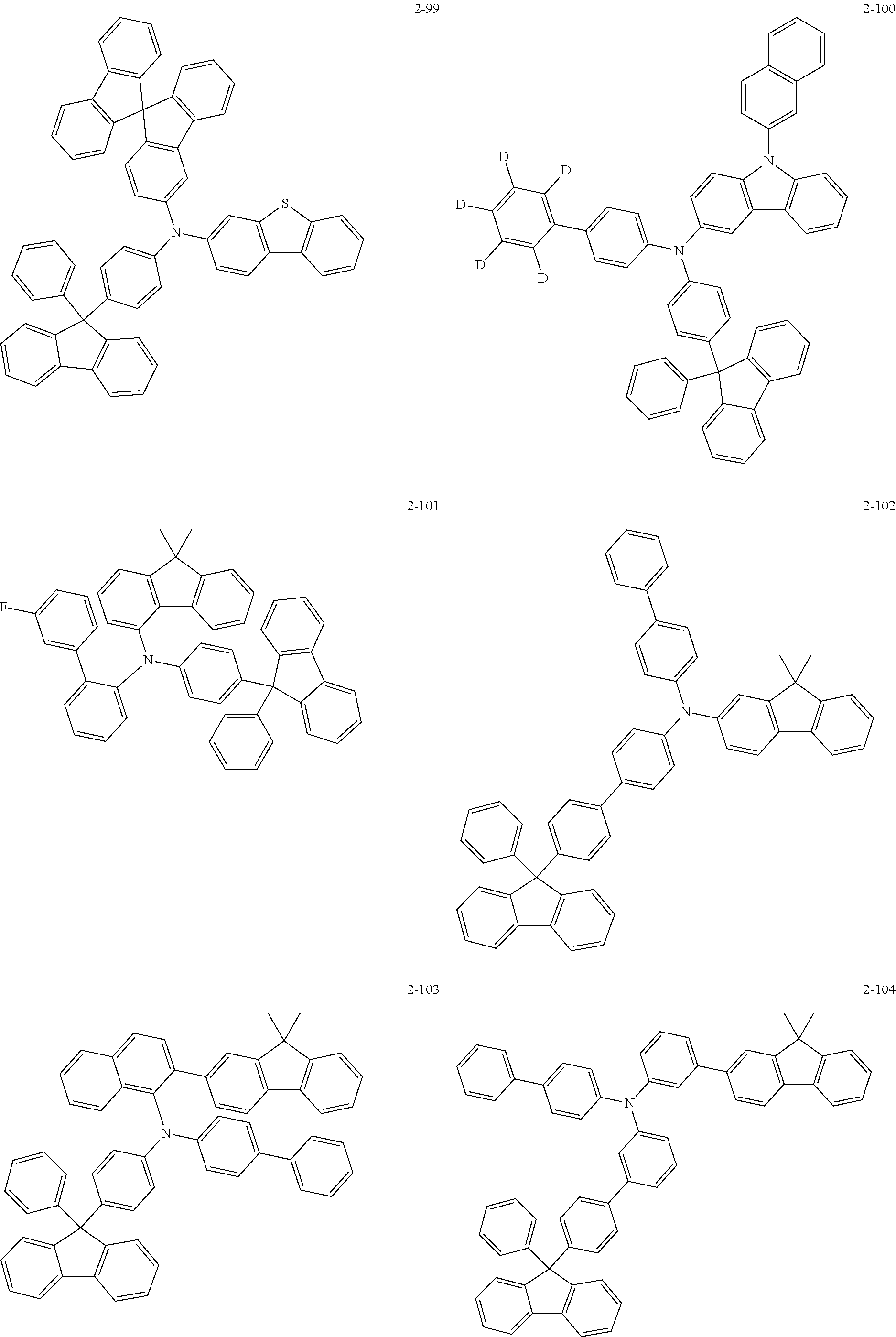

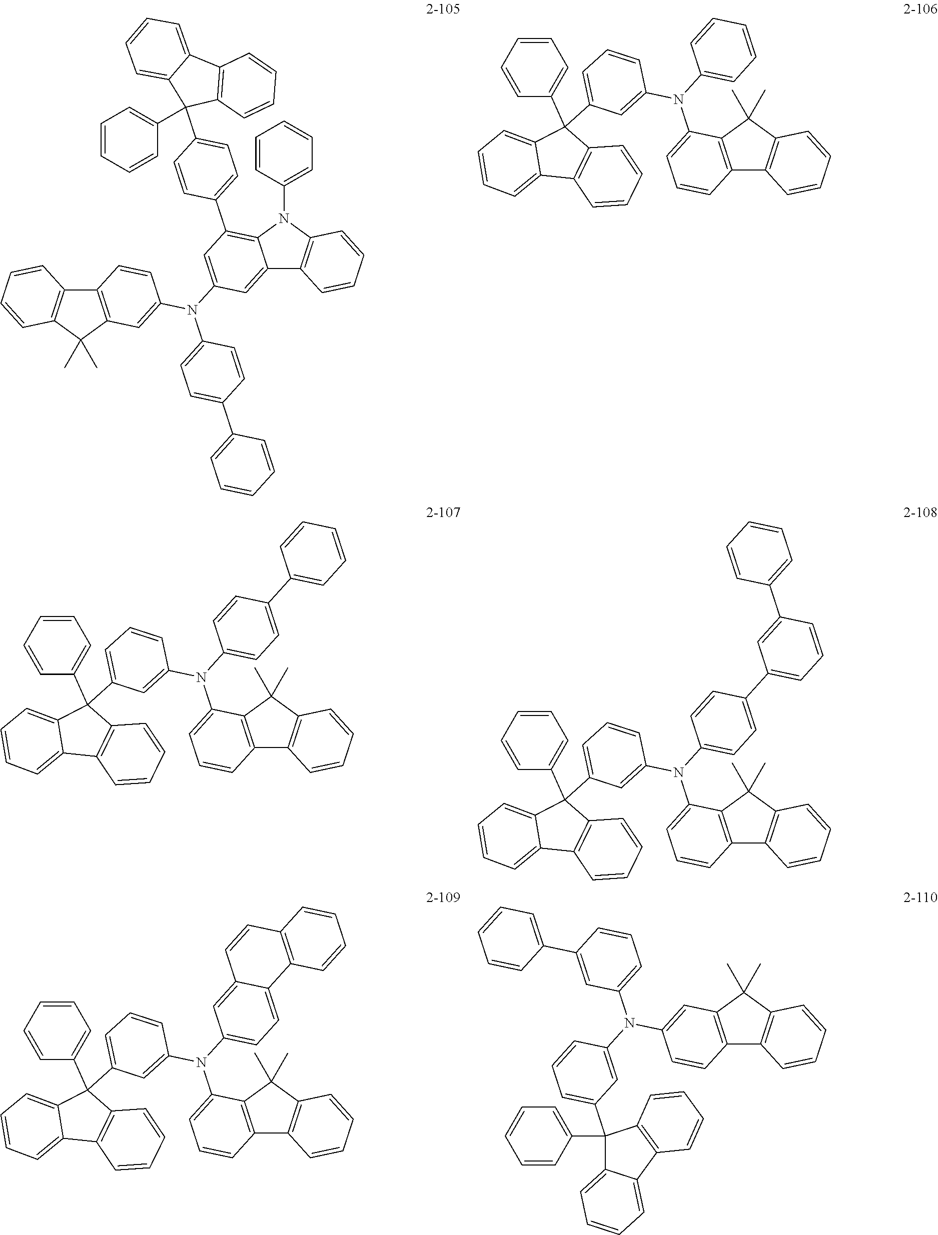

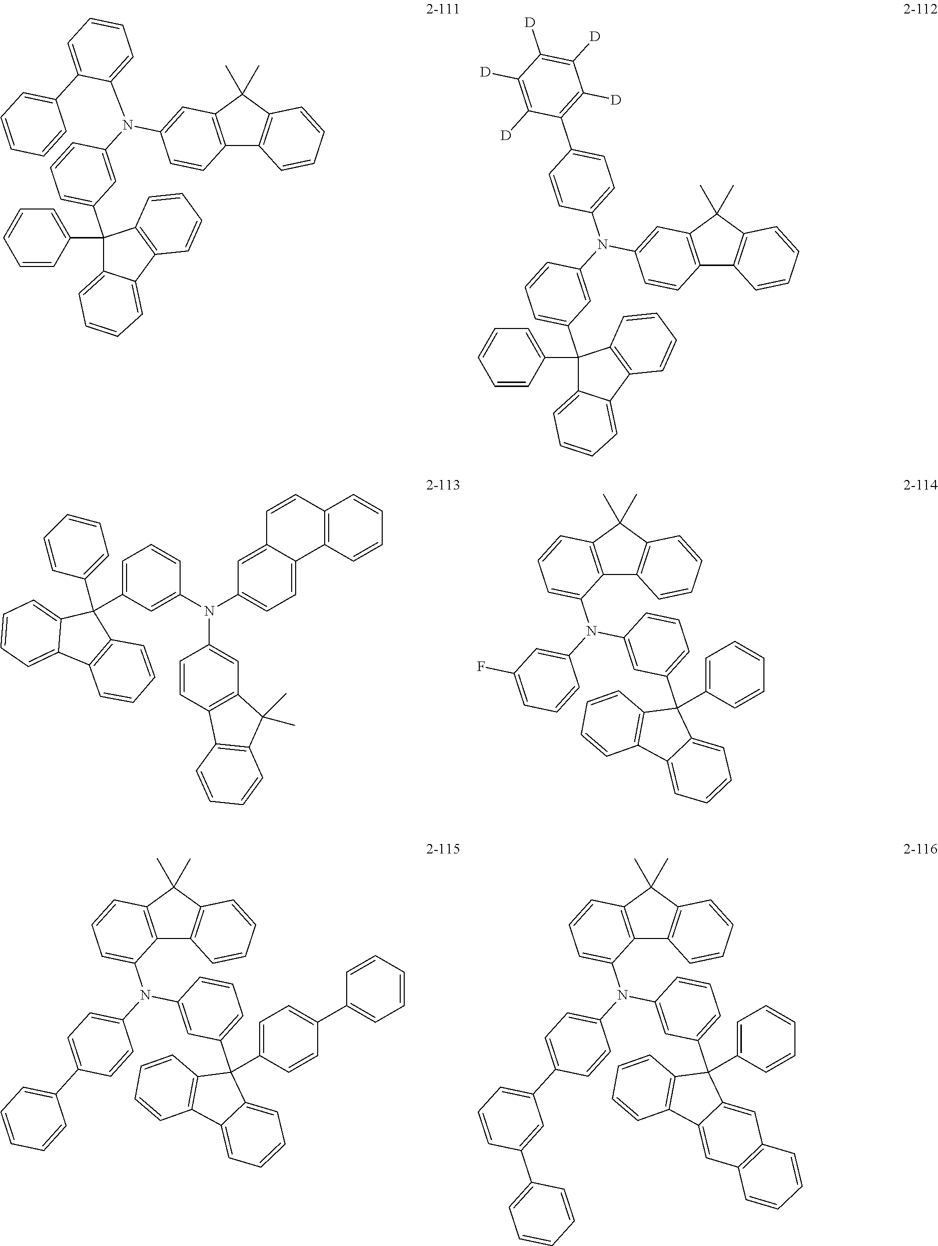

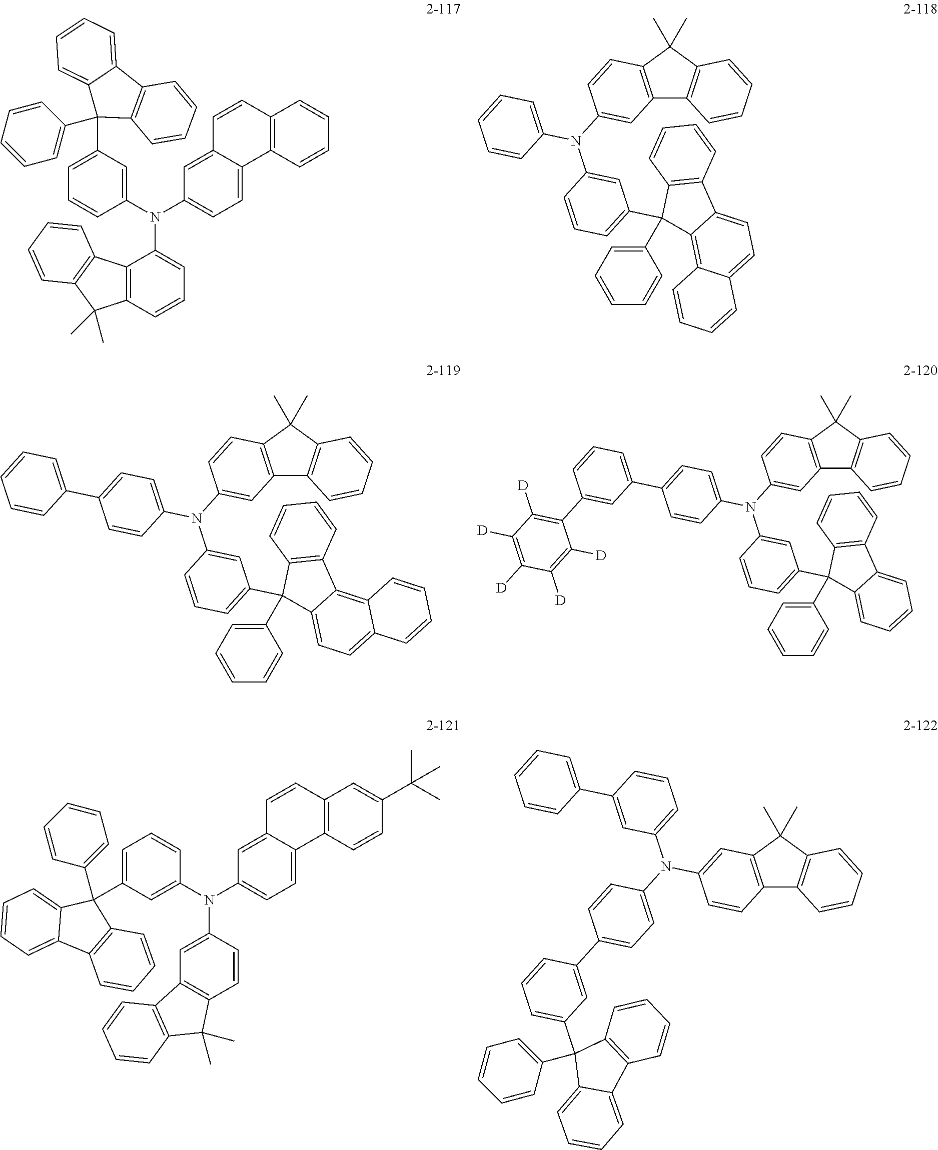

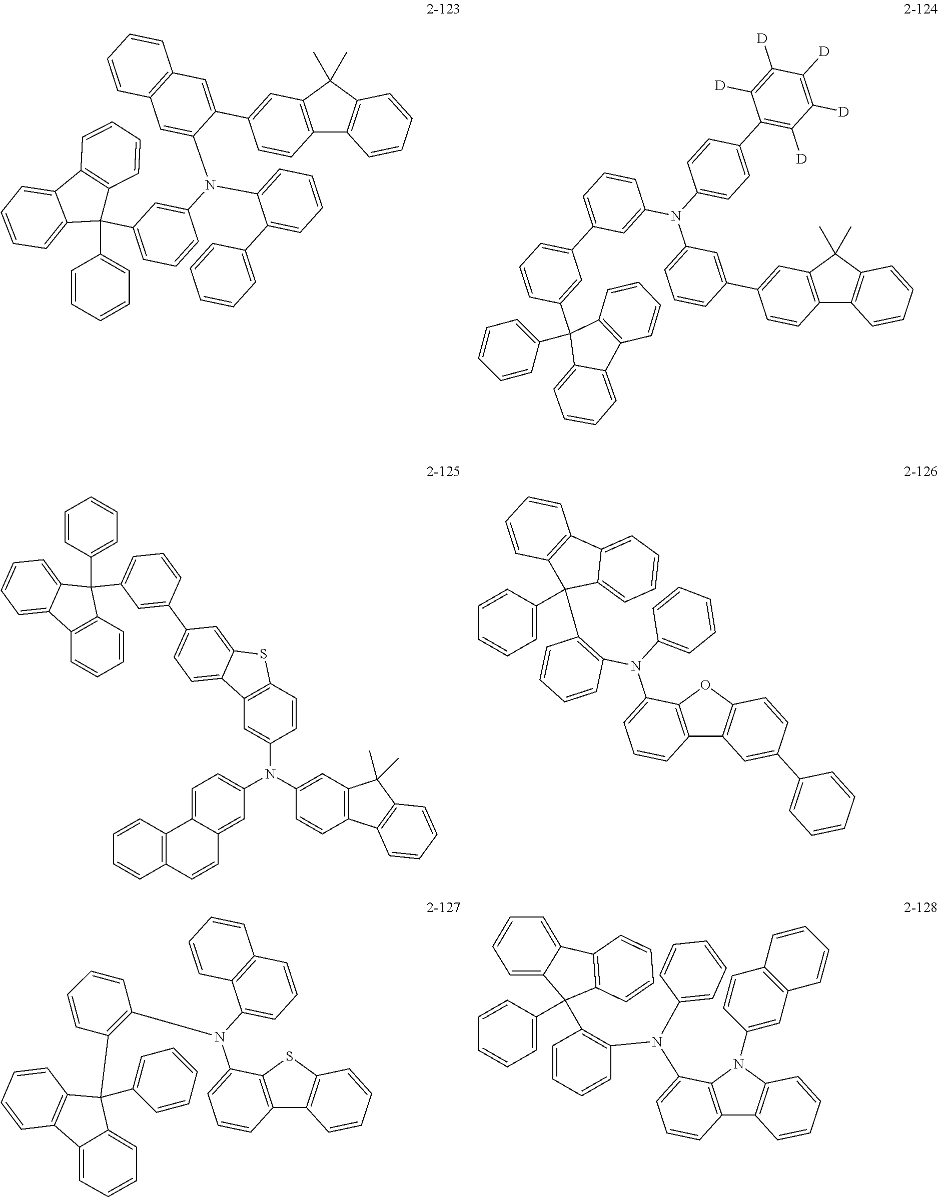

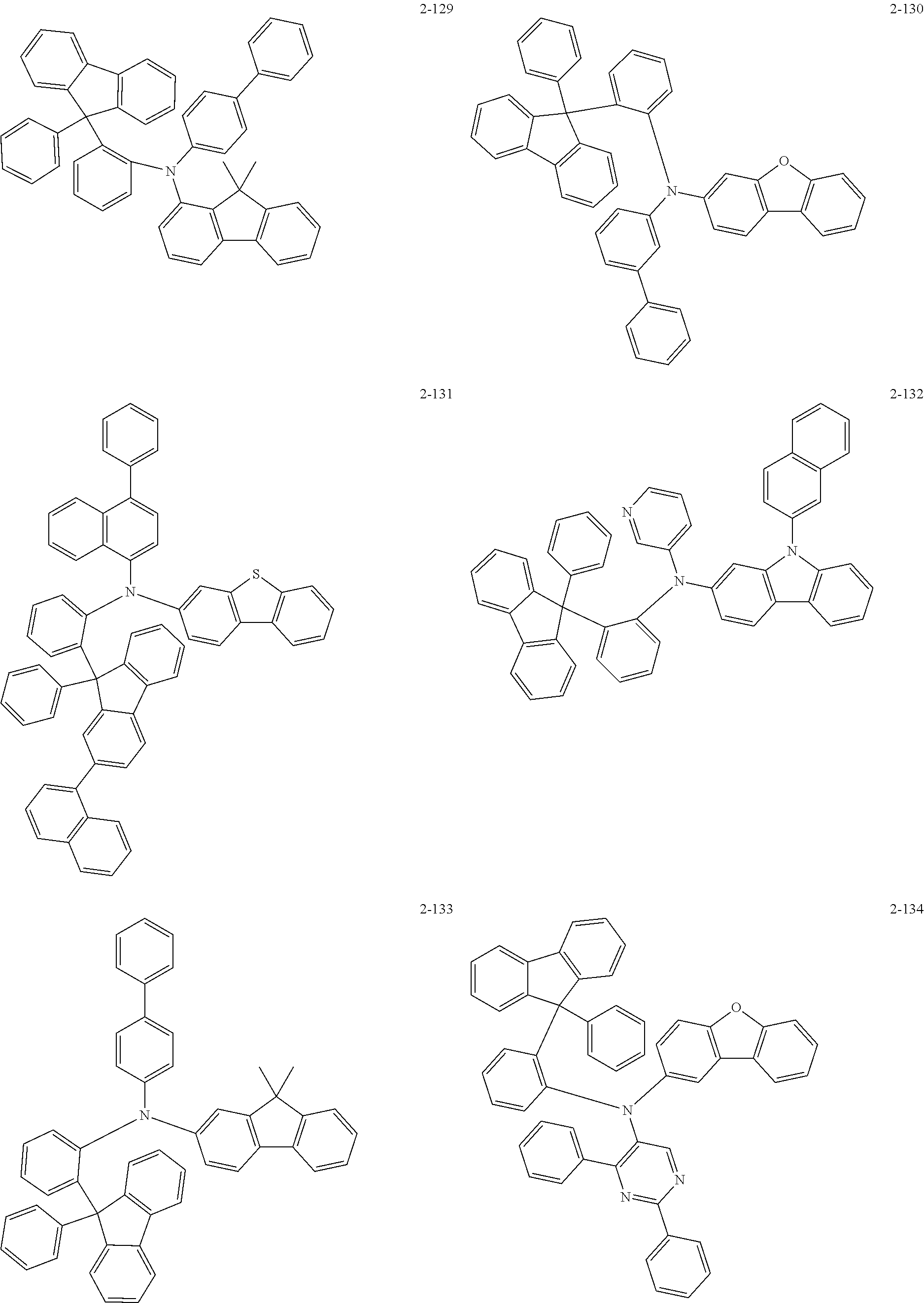

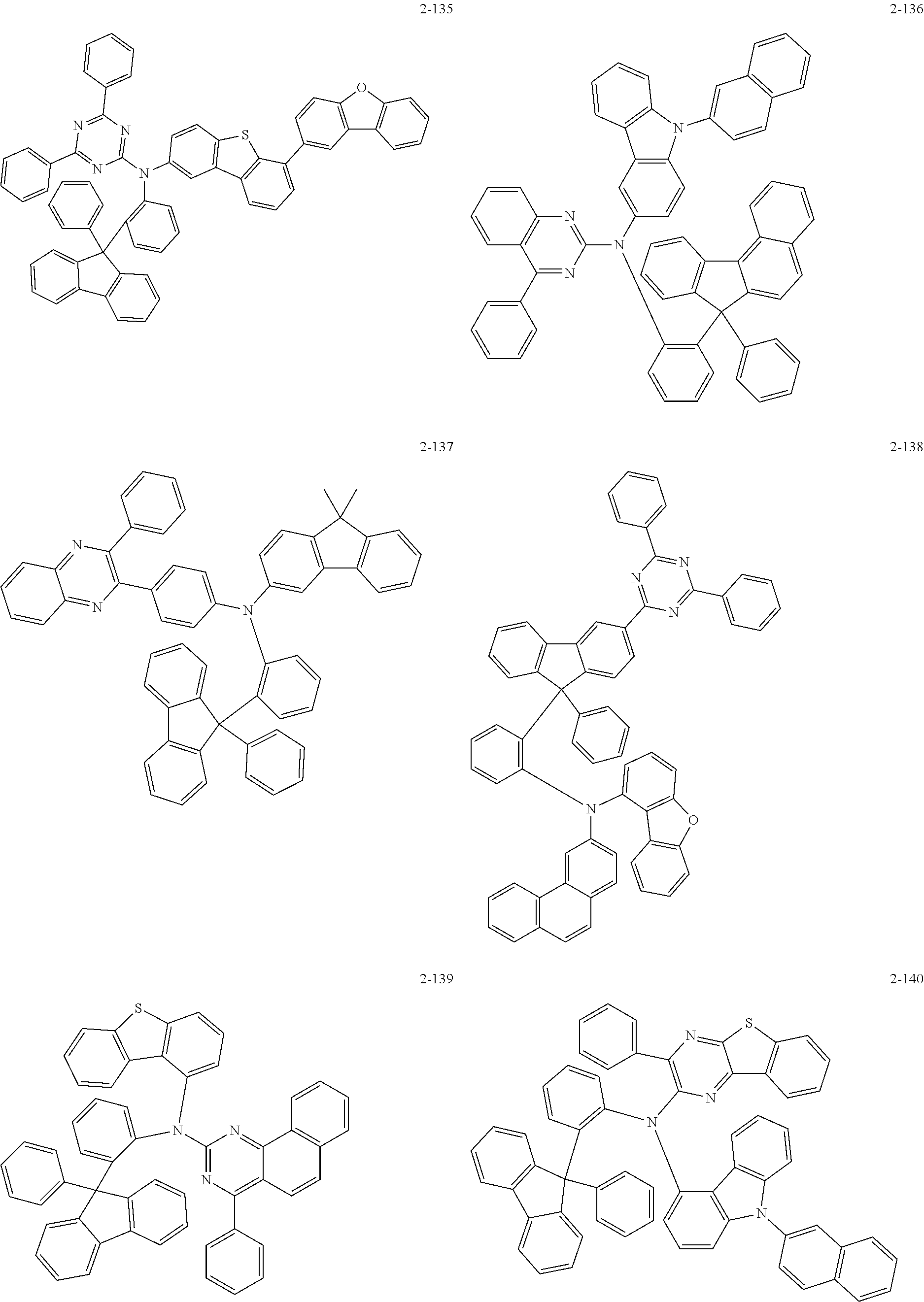

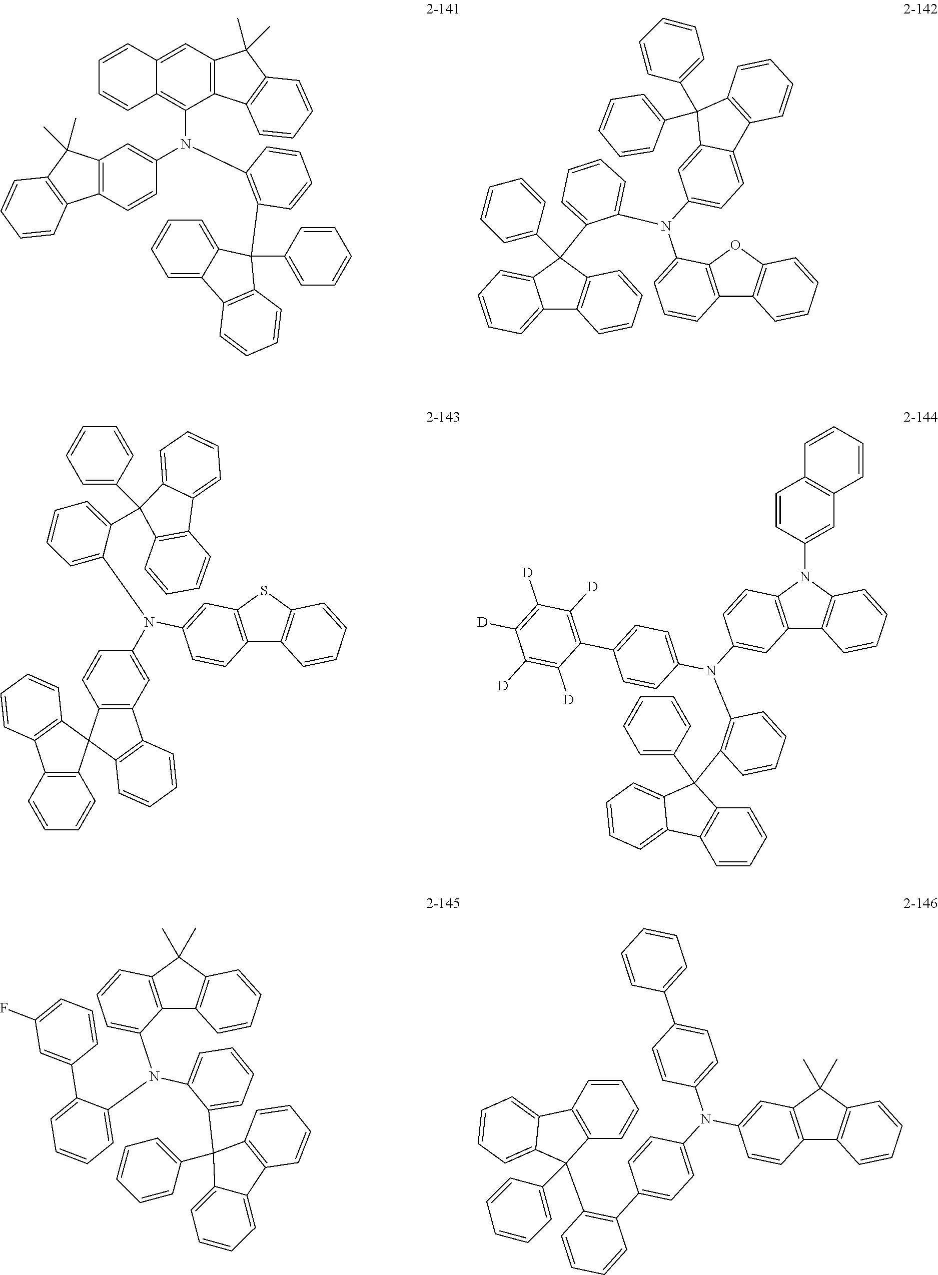

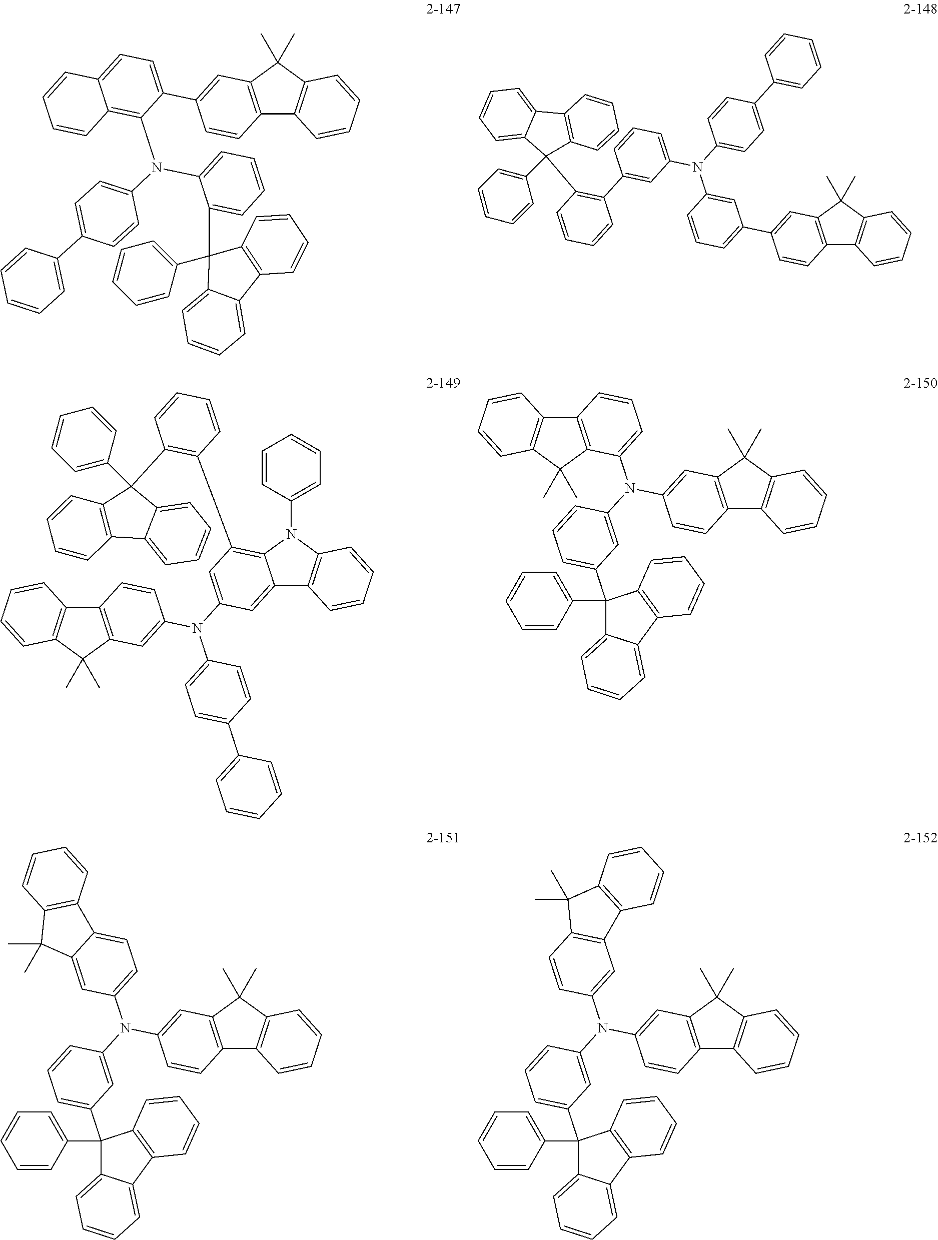

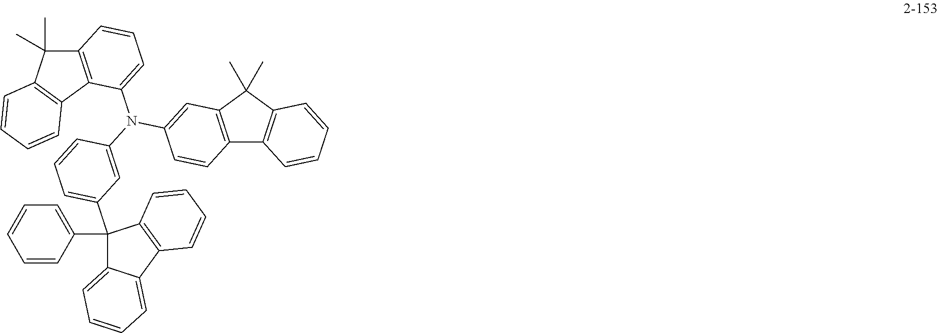 [SMILES: CC1(C)c2ccccc2-c2ccc(N(c3cccc(C4(c5ccccc5)c5ccccc5-c5ccccc54)c3)c3cccc4c3-c3ccccc3C4(C)C)cc21]

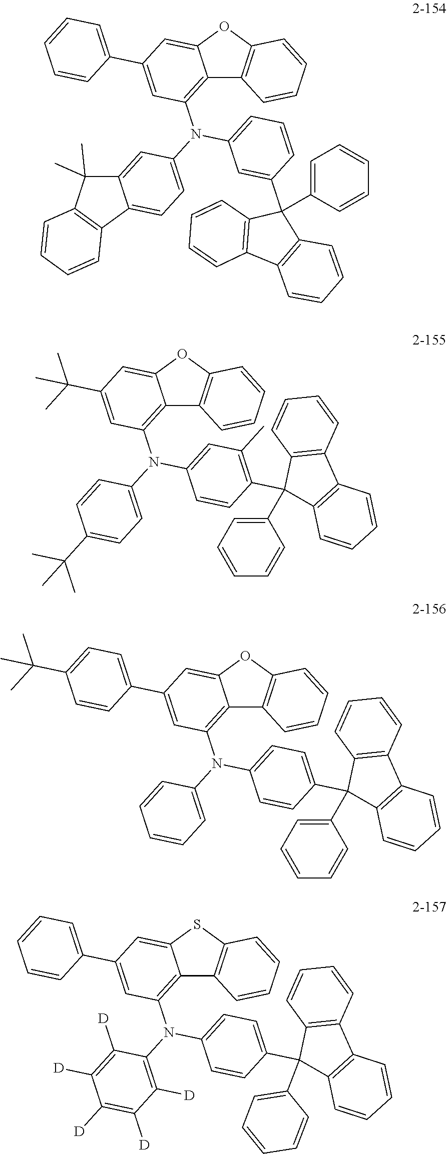 [SMILES: CC1(C)c2ccccc2-c2ccc(N(c3cccc(C4(c5ccccc5)c5ccccc5-c5ccccc54)c3)c3cc(-c4ccccc4)cc4oc5ccccc5c34)cc21 ; Cc1cc(N(c2ccc(C(C)(C)C)cc2)c2cc(C(C)(C)C)cc3oc4ccccc4c23)ccc1C1(c2ccccc2)c2ccccc2-c2ccccc21 ; CC(C)(C)c1ccc(-c2cc(N(c3ccccc3)c3ccc(C4(c5ccccc5)c5ccccc5-c5ccccc54)cc3)c3c(c2)oc2ccccc23)cc1 ; [2H]c1c([2H])c([2H])c(N(c2ccc(C3(c4ccccc4)c4ccccc4-c4ccccc43)cc2)c2cc(-c3ccccc3)cc3sc4ccccc4c23)c([2H])c1[2H]]

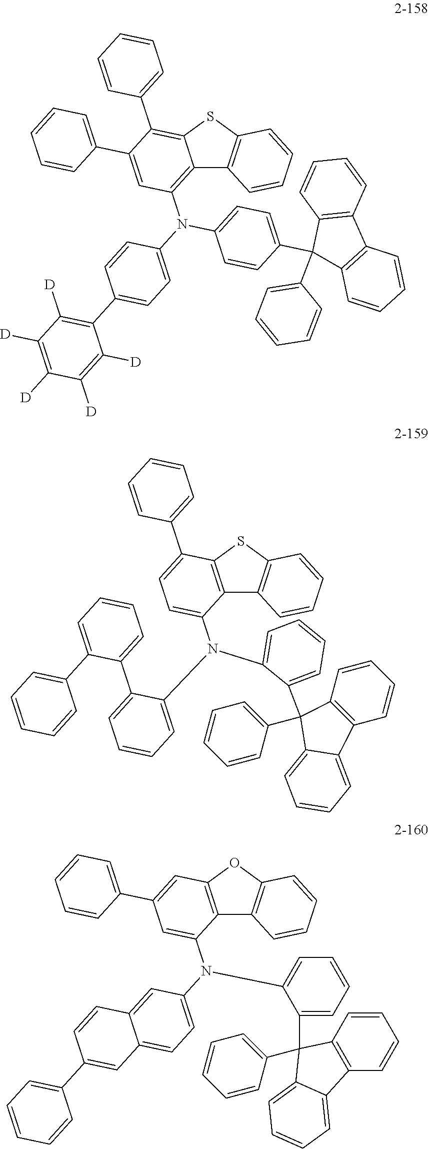 [SMILES: [2H]c1c([2H])c([2H])c(-c2ccc(N(c3ccc(C4(c5ccccc5)c5ccccc5-c5ccccc54)cc3)c3cc(-c4ccccc4)c(-c4ccccc4)c4sc5ccccc5c34)cc2)c([2H])c1[2H] ; c1ccc(-c2ccccc2-c2ccccc2N(c2ccccc2C2(c3ccccc3)c3ccccc3-c3ccccc32)c2ccc(-c3ccccc3)c3sc4ccccc4c23)cc1 ; c1ccc(-c2ccc3cc(N(c4ccccc4C4(c5ccccc5)c5ccccc5-c5ccccc54)c4cc(-c5ccccc5)cc5oc6ccccc6c45)ccc3c2)cc1]

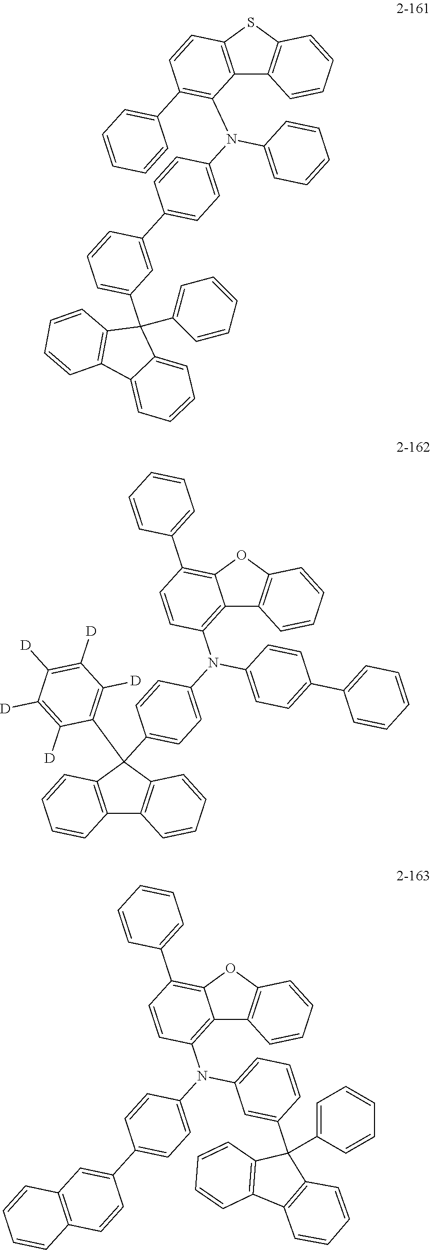 [SMILES: c1ccc(-c2ccc3sc4ccccc4c3c2N(c2ccccc2)c2ccc(-c3cccc(C4(c5ccccc5)c5ccccc5-c5ccccc54)c3)cc2)cc1 ; [2H]c1c([2H])c([2H])c(C2(c3ccc(N(c4ccc(-c5ccccc5)cc4)c4ccc(-c5ccccc5)c5oc6ccccc6c45)cc3)c3ccccc3-c3ccccc32)c([2H])c1[2H] ; c1ccc(-c2ccc(N(c3ccc(-c4ccc5ccccc5c4)cc3)c3cccc(C4(c5ccccc5)c5ccccc5-c5ccccc54)c3)c3c2oc2ccccc23)cc1]

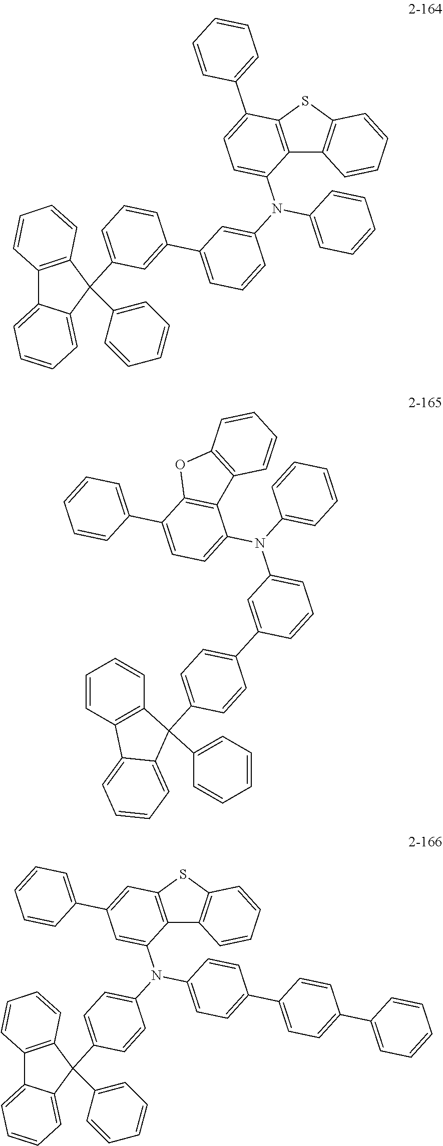 [SMILES: c1ccc(-c2ccc(N(c3ccccc3)c3cccc(-c4cccc(C5(c6ccccc6)c6ccccc6-c6ccccc65)c4)c3)c3c2sc2ccccc23)cc1 ; c1ccc(-c2ccc(N(c3ccccc3)c3cccc(-c4ccc(C5(c6ccccc6)c6ccccc6-c6ccccc65)cc4)c3)c3c2oc2ccccc23)cc1 ; c1ccc(-c2ccc(-c3ccc(N(c4ccc(C5(c6ccccc6)c6ccccc6-c6ccccc65)cc4)c4cc(-c5ccccc5)cc5sc6ccccc6c45)cc3)cc2)cc1]

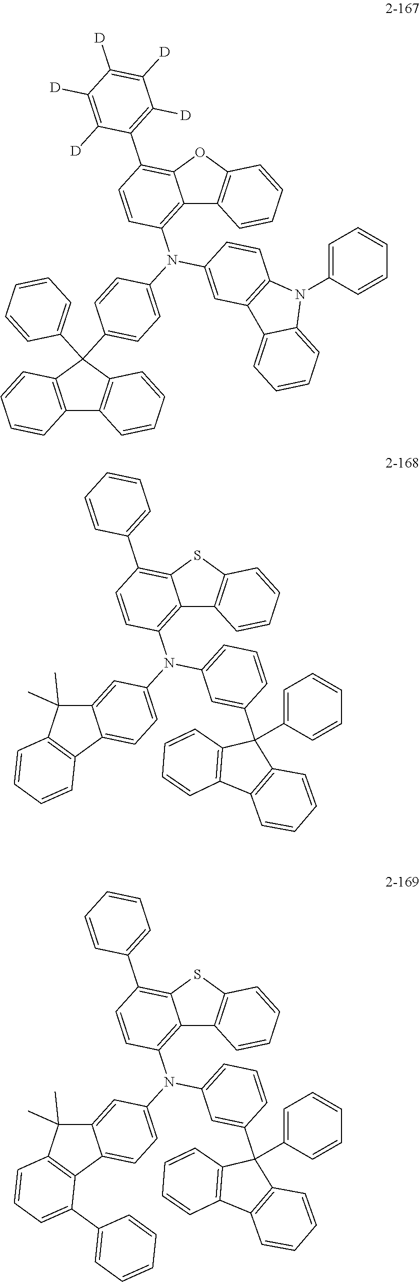

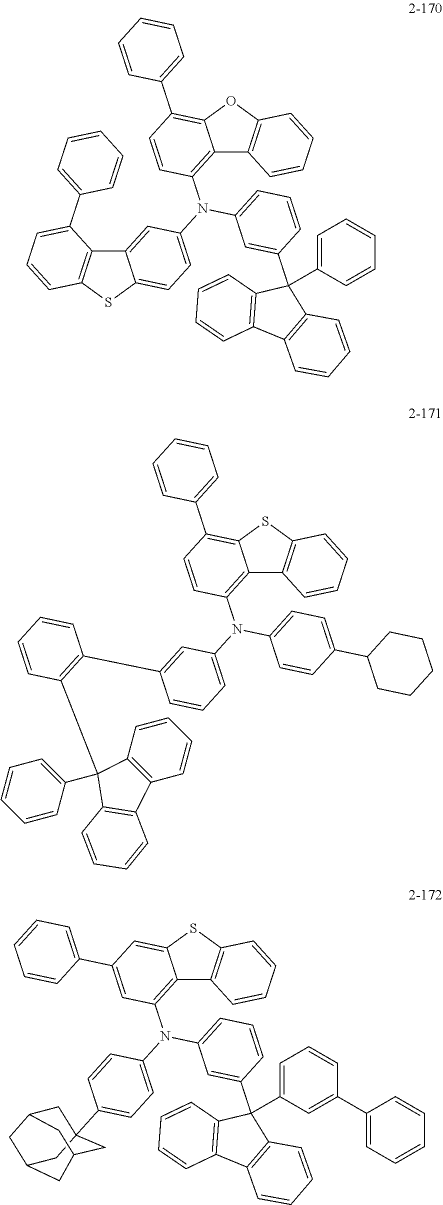 [SMILES: c1ccc(-c2ccc(N(c3cccc(C4(c5ccccc5)c5ccccc5-c5ccccc54)c3)c3ccc4sc5cccc(-c6ccccc6)c5c4c3)c3c2oc2ccccc23)cc1 ; c1ccc(-c2ccc(N(c3ccc(C4CCCCC4)cc3)c3cccc(-c4ccccc4C4(c5ccccc5)c5ccccc5-c5ccccc54)c3)c3c2sc2ccccc23)cc1 ; c1ccc(-c2cccc(C3(c4cccc(N(c5ccc(C67CC8CC(CC(C8)C6)C7)cc5)c5cc(-c6ccccc6)cc6sc7ccccc7c56)c4)c4ccccc4-c4ccccc43)c2)cc1]

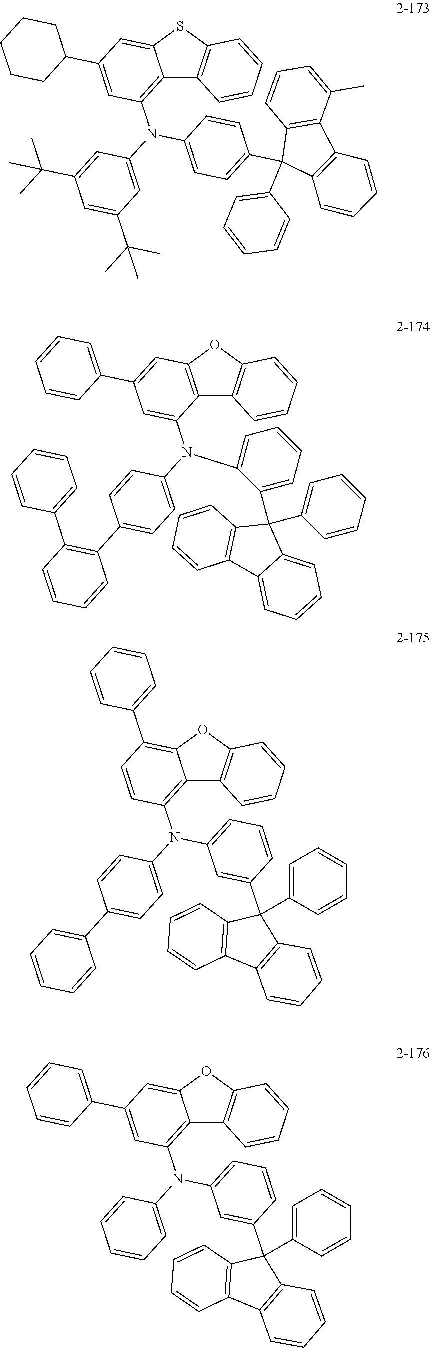 [SMILES: Cc1cccc2c1-c1ccccc1C2(c1ccccc1)c1ccc(N(c2cc(C(C)(C)C)cc(C(C)(C)C)c2)c2cc(C3CCCCC3)cc3sc4ccccc4c23)cc1 ; c1ccc(-c2cc(N(c3ccc(-c4ccccc4-c4ccccc4)cc3)c3ccccc3C3(c4ccccc4)c4ccccc4-c4ccccc43)c3c(c2)oc2ccccc23)cc1 ; c1ccc(-c2ccc(N(c3cccc(C4(c5ccccc5)c5ccccc5-c5ccccc54)c3)c3ccc(-c4ccccc4)c4oc5ccccc5c34)cc2)cc1 ; c1ccc(-c2cc(N(c3ccccc3)c3cccc(C4(c5ccccc5)c5ccccc5-c5ccccc54)c3)c3c(c2)oc2ccccc23)cc1]

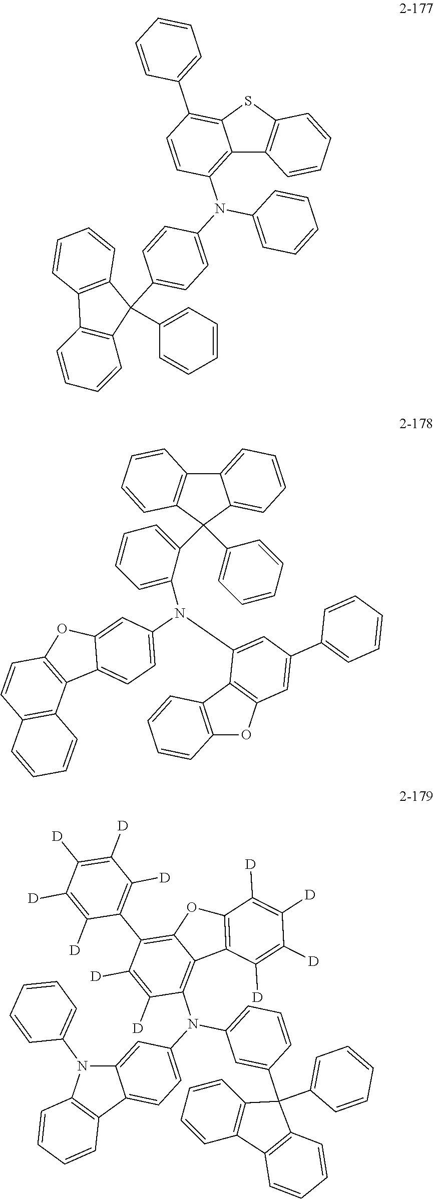 [SMILES: c1ccc(-c2ccc(N(c3ccccc3)c3ccc(C4(c5ccccc5)c5ccccc5-c5ccccc54)cc3)c3c2sc2ccccc23)cc1 ; c1ccc(-c2cc(N(c3ccc4c(c3)oc3ccc5ccccc5c34)c3ccccc3C3(c4ccccc4)c4ccccc4-c4ccccc43)c3c(c2)oc2ccccc23)cc1 ; [2H]c1c([2H])c([2H])c(-c2c([2H])c([2H])c(N(c3cccc(C4(c5ccccc5)c5ccccc5-c5ccccc54)c3)c3ccc4c5ccccc5n(-c5ccccc5)c4c3)c3c2oc2c([2H])c([2H])c([2H])c([2H])c23)c([2H])c1[2H]]

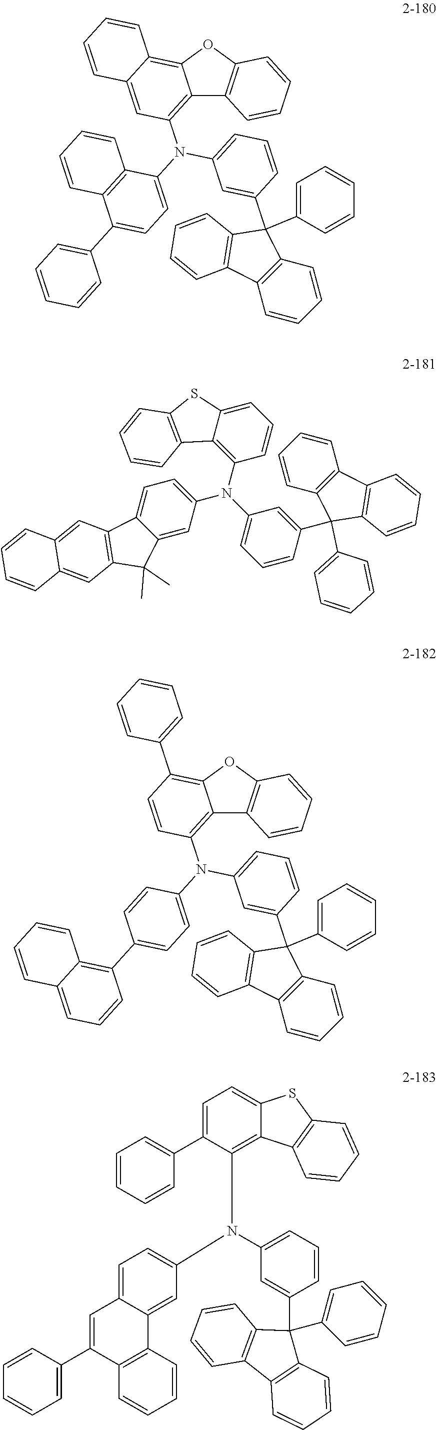 [SMILES: c1ccc(-c2ccc(N(c3cccc(C4(c5ccccc5)c5ccccc5-c5ccccc54)c3)c3cc4ccccc4c4oc5ccccc5c34)c3ccccc23)cc1 ; CC1(C)c2cc(N(c3cccc(C4(c5ccccc5)c5ccccc5-c5ccccc54)c3)c3cccc4sc5ccccc5c34)ccc2-c2cc3ccccc3cc21 ; c1ccc(-c2ccc(N(c3ccc(-c4cccc5ccccc45)cc3)c3cccc(C4(c5ccccc5)c5ccccc5-c5ccccc54)c3)c3c2oc2ccccc23)cc1 ; c1ccc(-c2ccc3sc4ccccc4c3c2N(c2cccc(C3(c4ccccc4)c4ccccc4-c4ccccc43)c2)c2ccc3cc(-c4ccccc4)c4ccccc4c3c2)cc1]

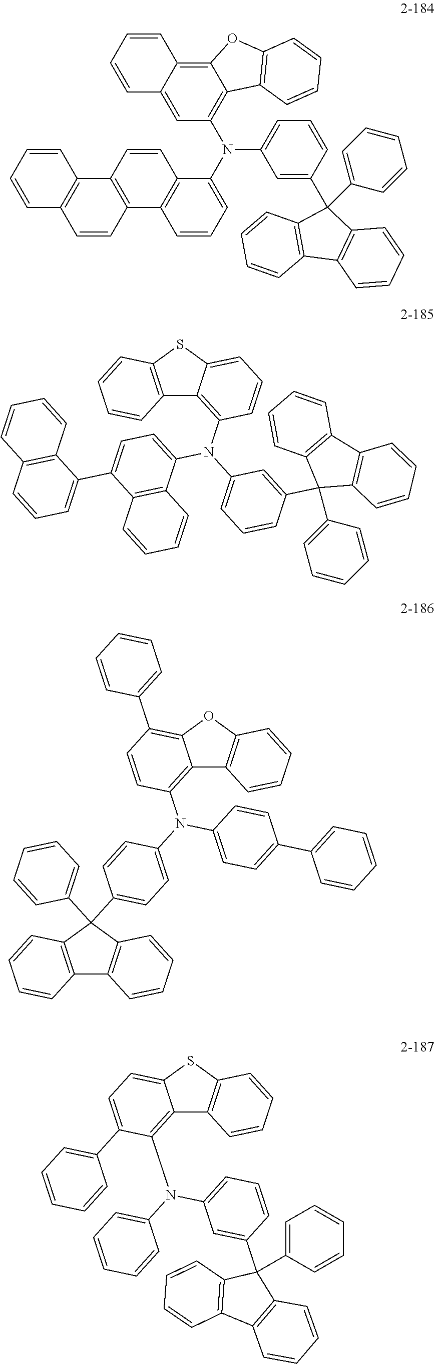 [SMILES: c1ccc(C2(c3cccc(N(c4cccc5c4ccc4c6ccccc6ccc54)c4cc5ccccc5c5oc6ccccc6c45)c3)c3ccccc3-c3ccccc32)cc1 ; c1ccc(C2(c3cccc(N(c4ccc(-c5cccc6ccccc56)c5ccccc45)c4cccc5sc6ccccc6c45)c3)c3ccccc3-c3ccccc32)cc1 ; c1ccc(-c2ccc(N(c3ccc(C4(c5ccccc5)c5ccccc5-c5ccccc54)cc3)c3ccc(-c4ccccc4)c4oc5ccccc5c34)cc2)cc1 ; c1ccc(-c2ccc3sc4ccccc4c3c2N(c2ccccc2)c2cccc(C3(c4ccccc4)c4ccccc4-c4ccccc43)c2)cc1]

As another example, the present invention provides an organic electronic element comprising an anode; a cathode; an organic material layer formed between the anode and the cathode; wherein the organic material layer includes an emitting layer, an hole transport layer formed between the anode and the emitting layer; an emitting auxiliary layer or an electron blocking layer (EBL) formed between the emitting layer and the hole transport layer; wherein the emitting auxiliary layer or the electron blocking layer comprises a compound represented by Formula (30).

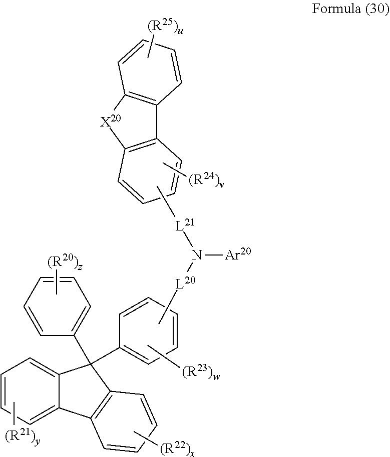

In Formula (30),
R²⁰, R²¹, R²², R²³, R²⁴, and R²⁵ are each independently selected from the group consisting of hydrogen; deuterium; halogen; a C₆-C₅₀ aryl group; a fluorenyl group; a C₂-C₅₀ heterocyclic group including at least one heteroatom of O, N, S, Si or P; a fused ring group of a C₃-C₅₀ aliphatic ring and a C₆-C₅₀ aromatic ring; a C₁-C₃₀ alkyl group; a C₂-C₂₀ alkenyl group; a C₂-C₂₀ alkynyl group; a C₁-C₃₀ alkoxyl group; a C₆-C₃₀ aryloxy group; or plurality of R²⁰, plurality of R²², plurality of R²³, plurality of R²⁴, and plurality of R²⁵ may be bonded to each other to form an aromatic ring or and heteroaromatic ring,
v is an integer of 0 to 3,
u, w, x and y are each independently an integer of 0 to 4,
Z is an integer of 0 to 5,
L²⁰ and L²¹ are each independently a single bond; a C₆-C₅₀ arylene group; a C₃-C₃₀ heteroarylene group;
Ar²⁰ is a C₆-C₃₀ aryl group; or a C₃-C₃₀ heteroarylene group;
X²⁰ is O, S, NR′ or CR′R″
R′ and R″ are each independently selected from the group of a C₁-C₃₀ alkyl group; a C₆-C₃₀ aryl group; a C₃-C₅₀ heterocyclic group including at least one heteroatom of O, N, S, Si, or P; and R′ and R″ may be bonded to each other to form a spiro.
wherein, the aryl group, fluorenyl group, arylene group, heterocyclic group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group and C₈-C₂₀ arylalkenyl group, wherein the substituents may be bonded to each other to form a ring, wherein the term ‘ring’ means a C₃-C₆₀ aliphatic ring or a C₆-C₆₀ aromatic ring or a C₂-C₆₀ heterocyclic group or a fused ring formed by the combination thereof and comprises a saturated or unsaturated ring.

In present invention, the compound represented by Formula (30) is represented by any of the Formulas (31) to (38)

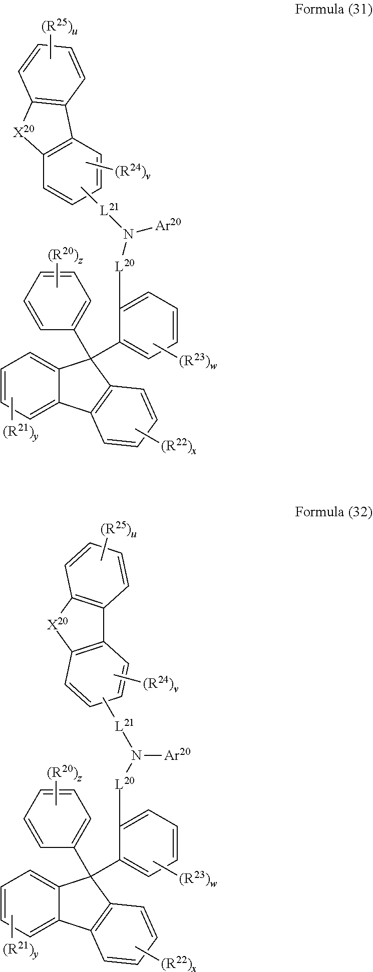

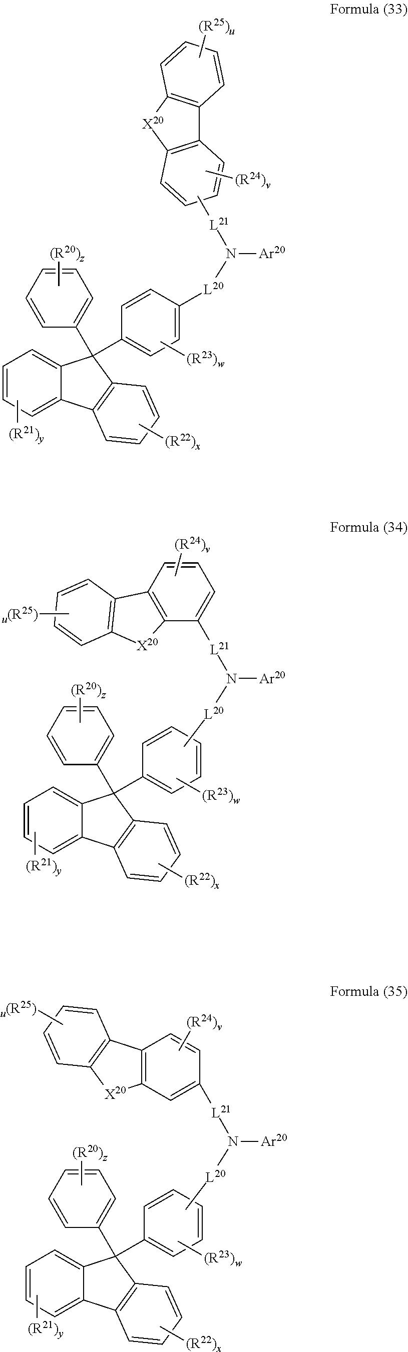

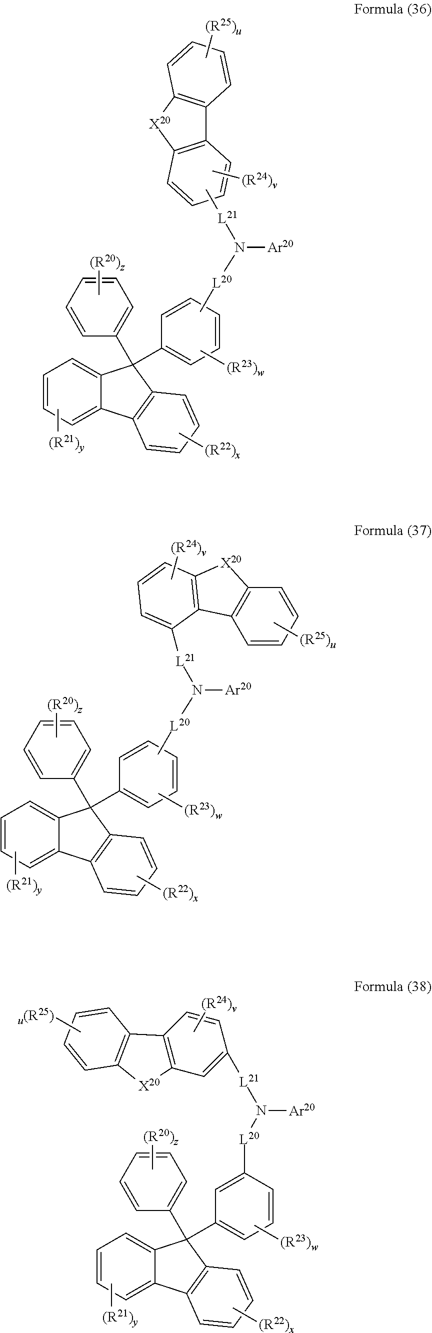

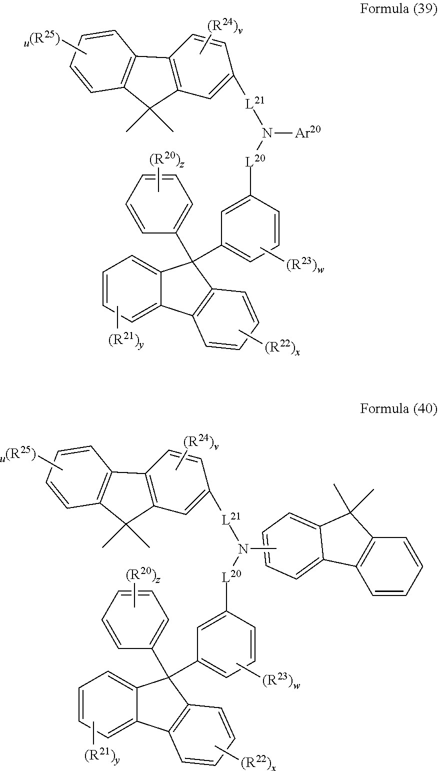

In Formulas (31) to (40),
R²⁰, R²¹, R²², R²³, R²⁴, R²⁵, L²⁰, L²¹, Ar²⁰ and X²⁰, u, v, w, x, y and z are the same as defined above.

In the present invention, the compound represented by Formula (30) comprises the following compounds.

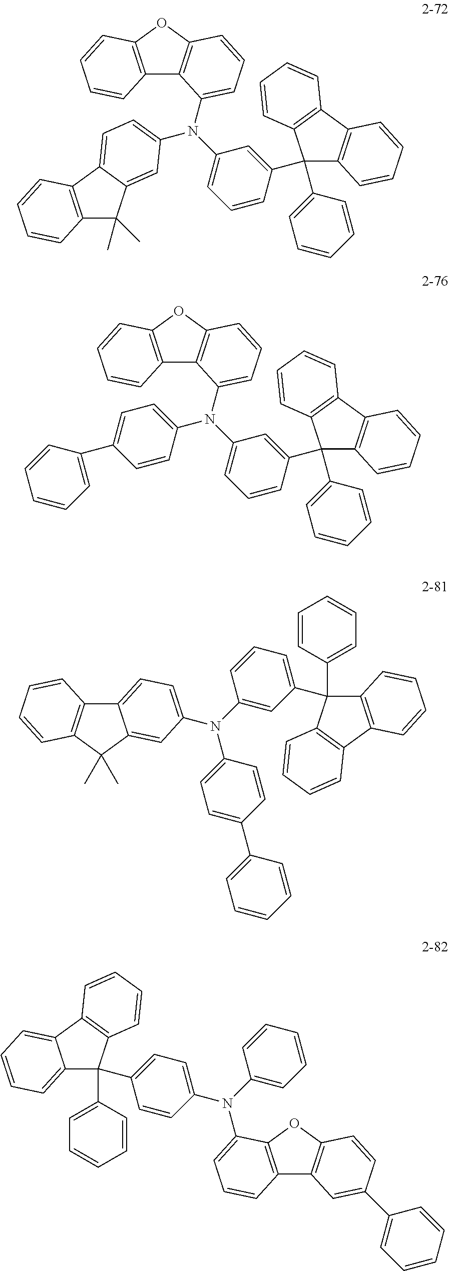 [SMILES: CC1(C)c2ccccc2-c2ccc(N(c3cccc(C4(c5ccccc5)c5ccccc5-c5ccccc54)c3)c3cccc4oc5ccccc5c34)cc21 ; c1ccc(-c2ccc(N(c3cccc(C4(c5ccccc5)c5ccccc5-c5ccccc54)c3)c3cccc4oc5ccccc5c34)cc2)cc1 ; CC1(C)c2ccccc2-c2ccc(N(c3ccc(-c4ccccc4)cc3)c3cccc(C4(c5ccccc5)c5ccccc5-c5ccccc54)c3)cc21 ; c1ccc(-c2ccc3oc4c(N(c5ccccc5)c5ccc(C6(c7ccccc7)c7ccccc7-c7ccccc76)cc5)cccc4c3c2)cc1]

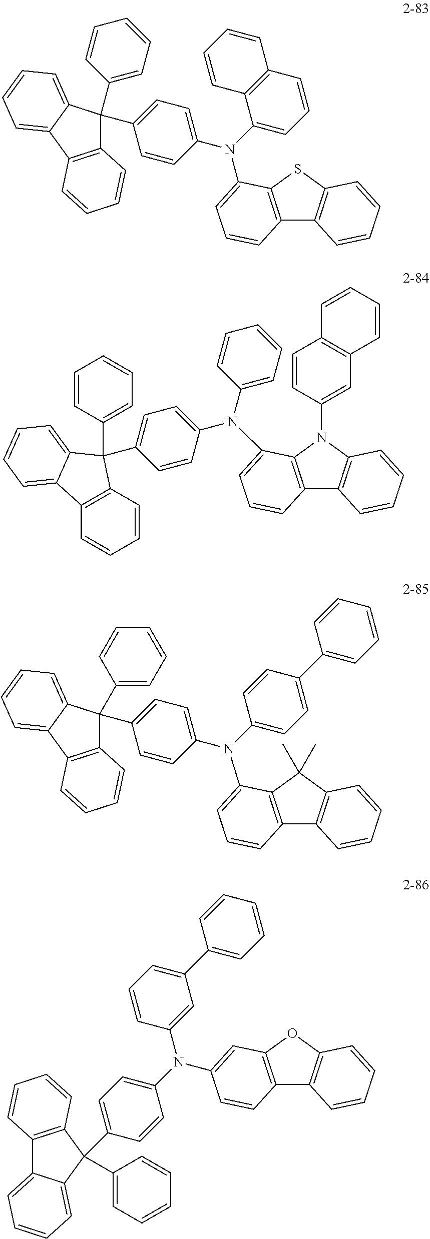

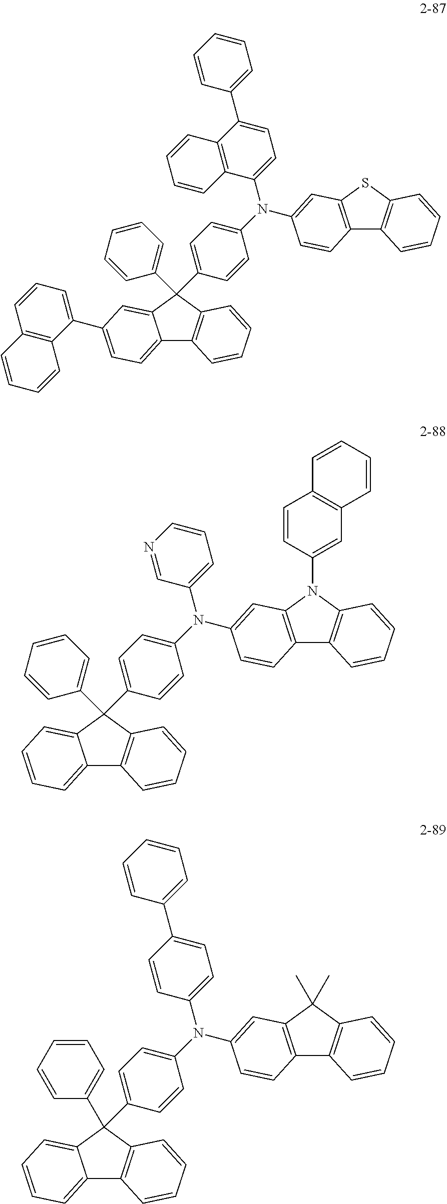 [SMILES: c1ccc(-c2ccc(N(c3ccc(C4(c5ccccc5)c5ccccc5-c5ccc(-c6cccc7ccccc67)cc54)cc3)c3ccc4c(c3)sc3ccccc34)c3ccccc23)cc1 ; c1ccc(C2(c3ccc(N(c4cccnc4)c4ccc5c6ccccc6n(-c6ccc7ccccc7c6)c5c4)cc3)c3ccccc3-c3ccccc32)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

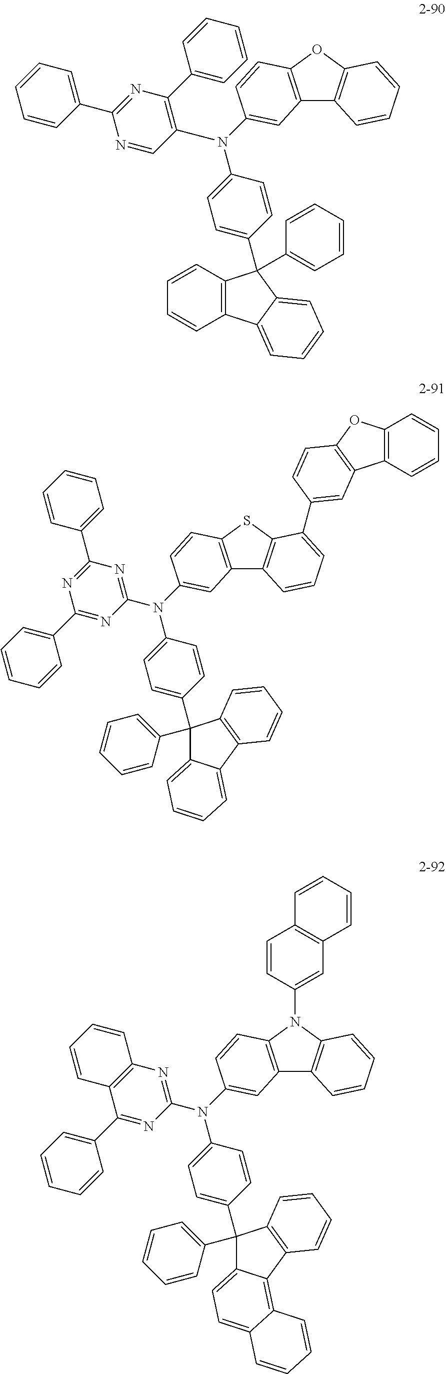 [SMILES: c1ccc(-c2ncc(N(c3ccc(C4(c5ccccc5)c5ccccc5-c5ccccc54)cc3)c3ccc4oc5ccccc5c4c3)c(-c3ccccc3)n2)cc1 ; c1ccc(-c2nc(-c3ccccc3)nc(N(c3ccc(C4(c5ccccc5)c5ccccc5-c5ccccc54)cc3)c3ccc4sc5c(-c6ccc7oc8ccccc8c7c6)cccc5c4c3)n2)cc1 ; c1ccc(-c2nc(N(c3ccc(C4(c5ccccc5)c5ccccc5-c5c4ccc4ccccc54)cc3)c3ccc4c(c3)c3ccccc3n4-c3ccc4ccccc4c3)nc3ccccc23)cc1]

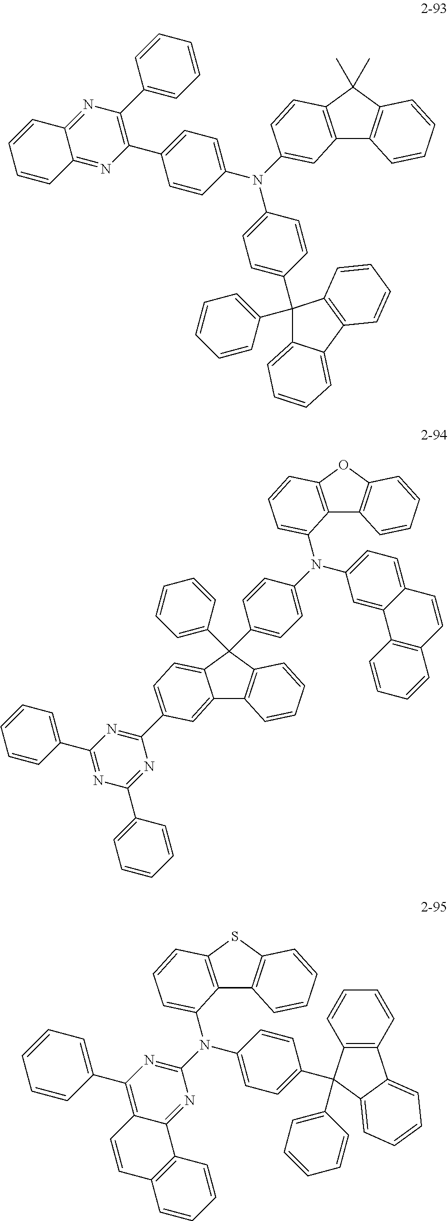 [SMILES: CC1(C)c2ccccc2-c2cc(N(c3ccc(-c4nc5ccccc5nc4-c4ccccc4)cc3)c3ccc(C4(c5ccccc5)c5ccccc5-c5ccccc54)cc3)ccc21 ; c1ccc(-c2nc(-c3ccccc3)nc(-c3ccc4c(c3)-c3ccccc3C4(c3ccccc3)c3ccc(N(c4ccc5ccc6ccccc6c5c4)c4cccc5oc6ccccc6c45)cc3)n2)cc1 ; c1ccc(-c2nc(N(c3ccc(C4(c5ccccc5)c5ccccc5-c5ccccc54)cc3)c3cccc4sc5ccccc5c34)nc3c2ccc2ccccc23)cc1]

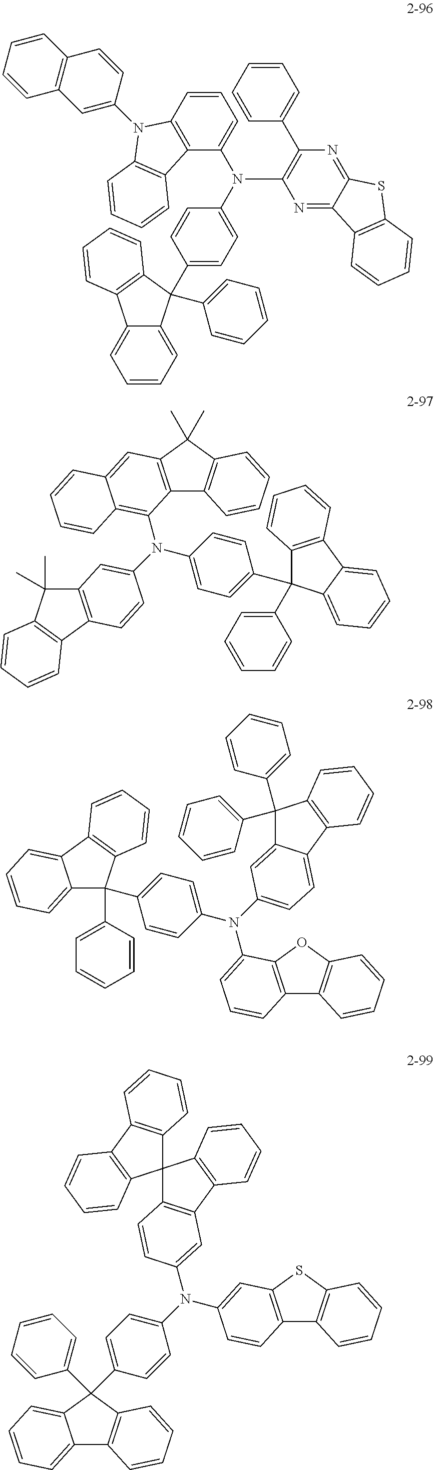

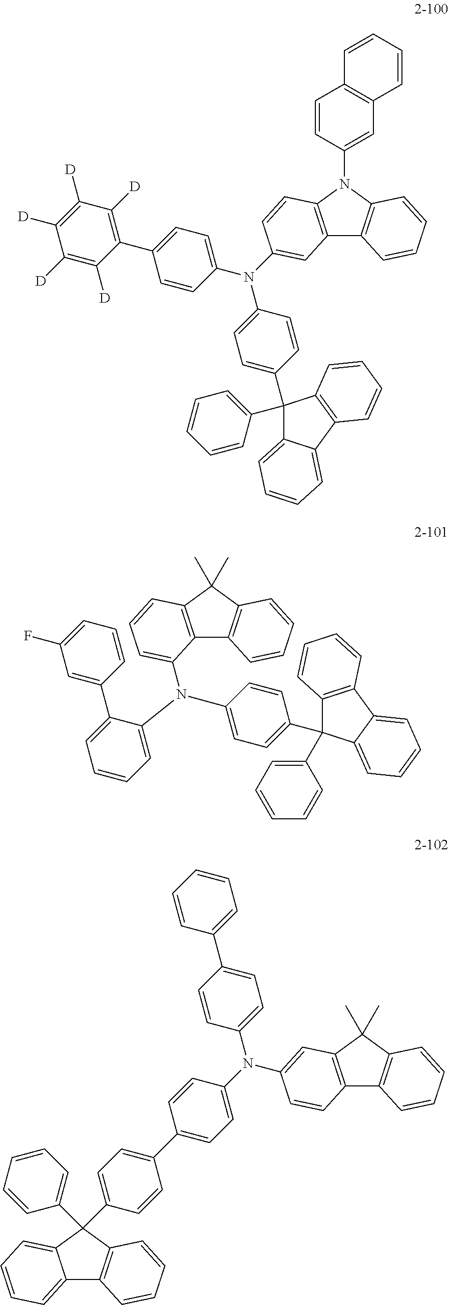 [SMILES: [2H]c1c([2H])c([2H])c(-c2ccc(N(c3ccc(C4(c5ccccc5)c5ccccc5-c5ccccc54)cc3)c3ccc4c(c3)c3ccccc3n4-c3ccc4ccccc4c3)cc2)c([2H])c1[2H] ; CC1(C)c2ccccc2-c2c(N(c3ccc(C4(c5ccccc5)c5ccccc5-c5ccccc54)cc3)c3ccccc3-c3cccc(F)c3)cccc21 ; CC1(C)c2ccccc2-c2ccc(N(c3ccc(-c4ccccc4)cc3)c3ccc(-c4ccc(C5(c6ccccc6)c6ccccc6-c6ccccc65)cc4)cc3)cc21]

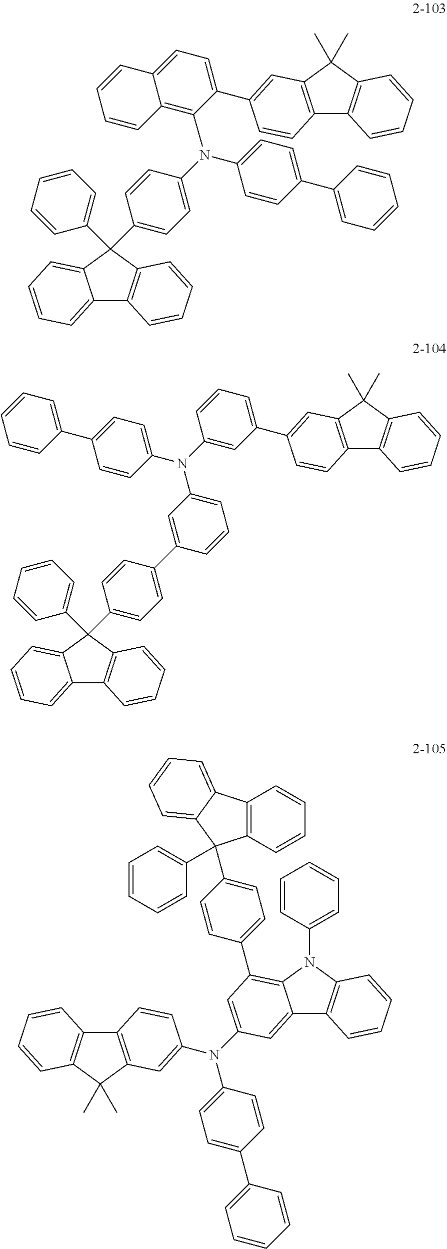 [SMILES: CC1(C)c2ccccc2-c2ccc(-c3ccc4ccccc4c3N(c3ccc(-c4ccccc4)cc3)c3ccc(C4(c5ccccc5)c5ccccc5-c5ccccc54)cc3)cc21 ; CC1(C)c2ccccc2-c2ccc(-c3cccc(N(c4ccc(-c5ccccc5)cc4)c4cccc(-c5ccc(C6(c7ccccc7)c7ccccc7-c7ccccc76)cc5)c4)c3)cc21 ; CC1(C)c2ccccc2-c2ccc(N(c3ccc(-c4ccccc4)cc3)c3cc(-c4ccc(C5(c6ccccc6)c6ccccc6-c6ccccc65)cc4)c4c(c3)c3ccccc3n4-c3ccccc3)cc21]

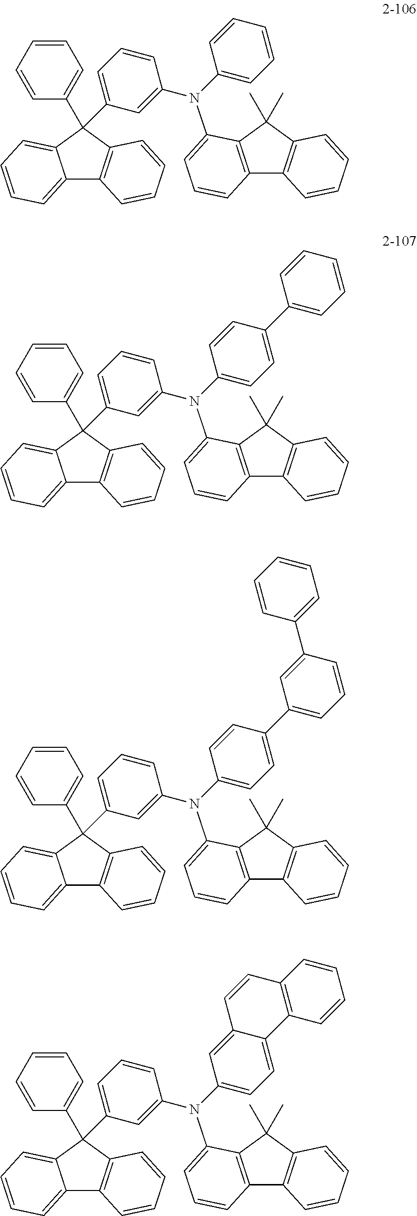 [SMILES: CC1(C)c2ccccc2-c2cccc(N(c3ccccc3)c3cccc(C4(c5ccccc5)c5ccccc5-c5ccccc54)c3)c21 ; CC1(C)c2ccccc2-c2cccc(N(c3ccc(-c4ccccc4)cc3)c3cccc(C4(c5ccccc5)c5ccccc5-c5ccccc54)c3)c21 ; CC1(C)c2ccccc2-c2cccc(N(c3ccc(-c4cccc(-c5ccccc5)c4)cc3)c3cccc(C4(c5ccccc5)c5ccccc5-c5ccccc54)c3)c21 ; CC1(C)c2ccccc2-c2cccc(N(c3cccc(C4(c5ccccc5)c5ccccc5-c5ccccc54)c3)c3ccc4c(ccc5ccccc54)c3)c21]

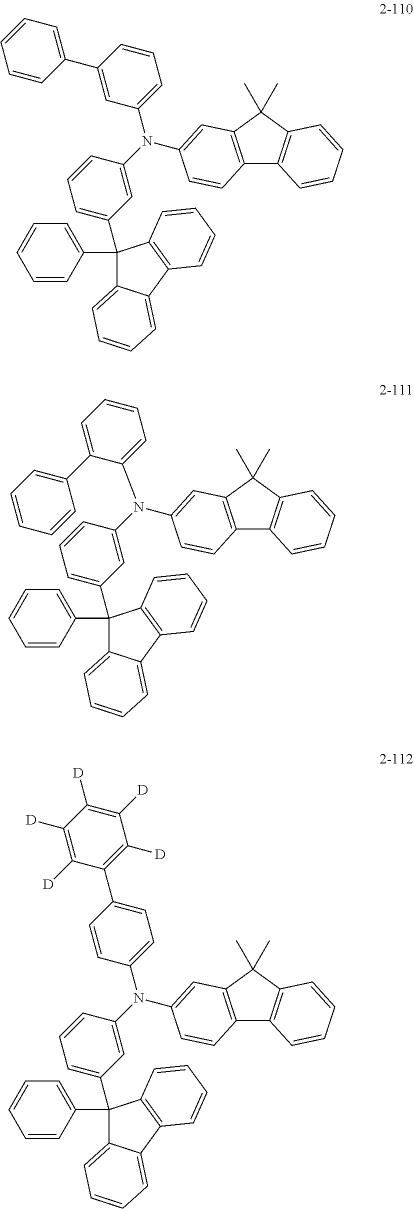

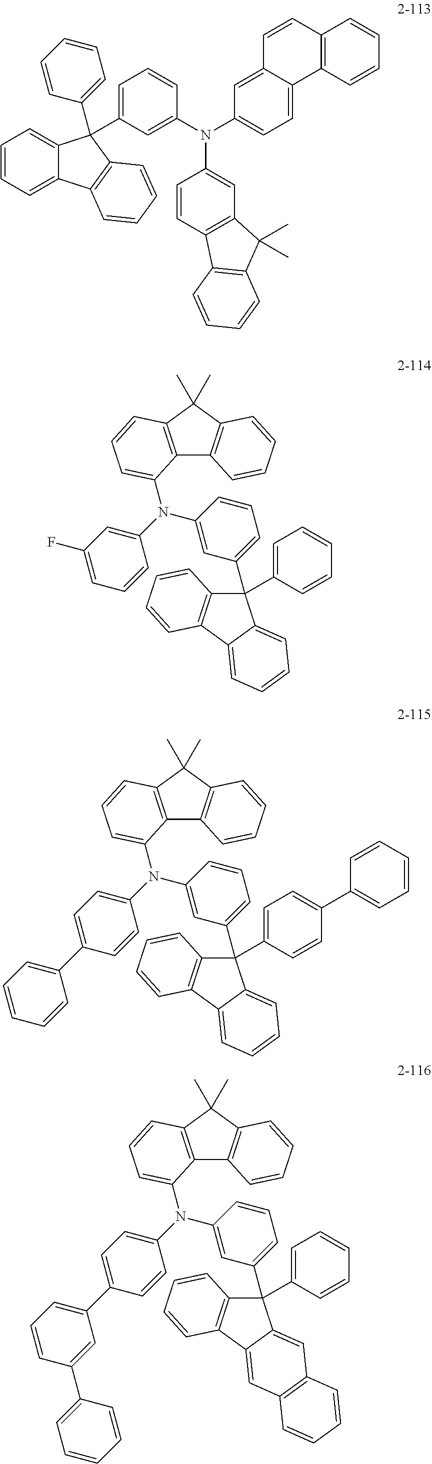

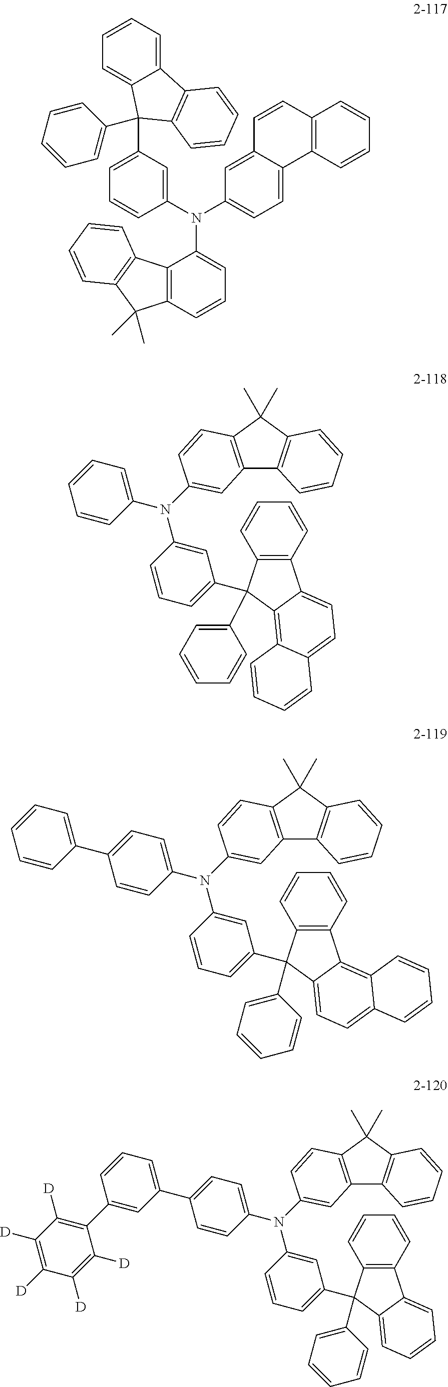 [SMILES: CC1(C)c2ccccc2-c2c(N(c3cccc(C4(c5ccccc5)c5ccccc5-c5ccccc54)c3)c3ccc4c(ccc5ccccc54)c3)cccc21 ; CC1(C)c2ccccc2-c2cc(N(c3ccccc3)c3cccc(C4(c5ccccc5)c5ccccc5-c5ccc6ccccc6c54)c3)ccc21 ; CC1(C)c2ccccc2-c2cc(N(c3ccc(-c4ccccc4)cc3)c3cccc(C4(c5ccccc5)c5ccccc5-c5c4ccc4ccccc54)c3)ccc21 ; [2H]c1c([2H])c([2H])c(-c2cccc(-c3ccc(N(c4cccc(C5(c6ccccc6)c6ccccc6-c6ccccc65)c4)c4ccc5c(c4)-c4ccccc4C5(C)C)cc3)c2)c([2H])c1[2H]]

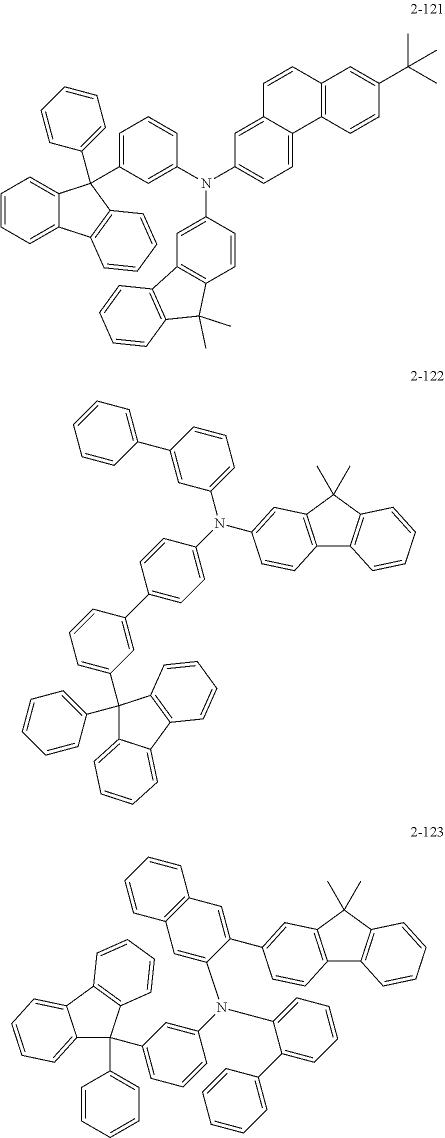 [SMILES: CC(C)(C)c1ccc2c(ccc3cc(N(c4cccc(C5(c6ccccc6)c6ccccc6-c6ccccc65)c4)c4ccc5c(c4)-c4ccccc4C5(C)C)ccc32)c1 ; CC1(C)c2ccccc2-c2ccc(N(c3ccc(-c4cccc(C5(c6ccccc6)c6ccccc6-c6ccccc65)c4)cc3)c3cccc(-c4ccccc4)c3)cc21 ; CC1(C)c2ccccc2-c2ccc(-c3cc4ccccc4cc3N(c3cccc(C4(c5ccccc5)c5ccccc5-c5ccccc54)c3)c3ccccc3-c3ccccc3)cc21]

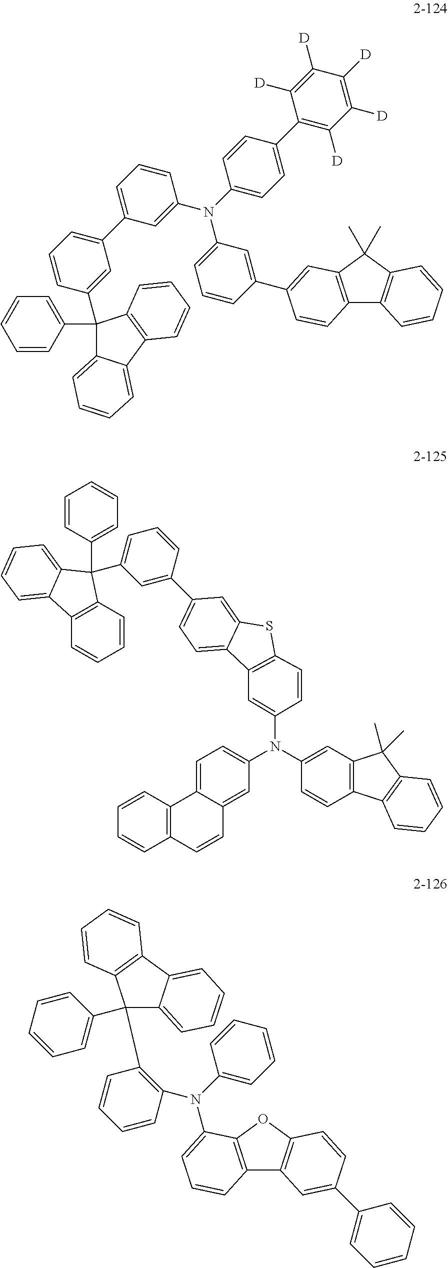 [SMILES: [2H]c1c([2H])c([2H])c(-c2ccc(N(c3cccc(-c4cccc(C5(c6ccccc6)c6ccccc6-c6ccccc65)c4)c3)c3cccc(-c4ccc5c(c4)C(C)(C)c4ccccc4-5)c3)cc2)c([2H])c1[2H] ; CC1(C)c2ccccc2-c2ccc(N(c3ccc4c(ccc5ccccc54)c3)c3ccc4sc5cc(-c6cccc(C7(c8ccccc8)c8ccccc8-c8ccccc87)c6)ccc5c4c3)cc21 ; c1ccc(-c2ccc3oc4c(N(c5ccccc5)c5ccccc5C5(c6ccccc6)c6ccccc6-c6ccccc65)cccc4c3c2)cc1]

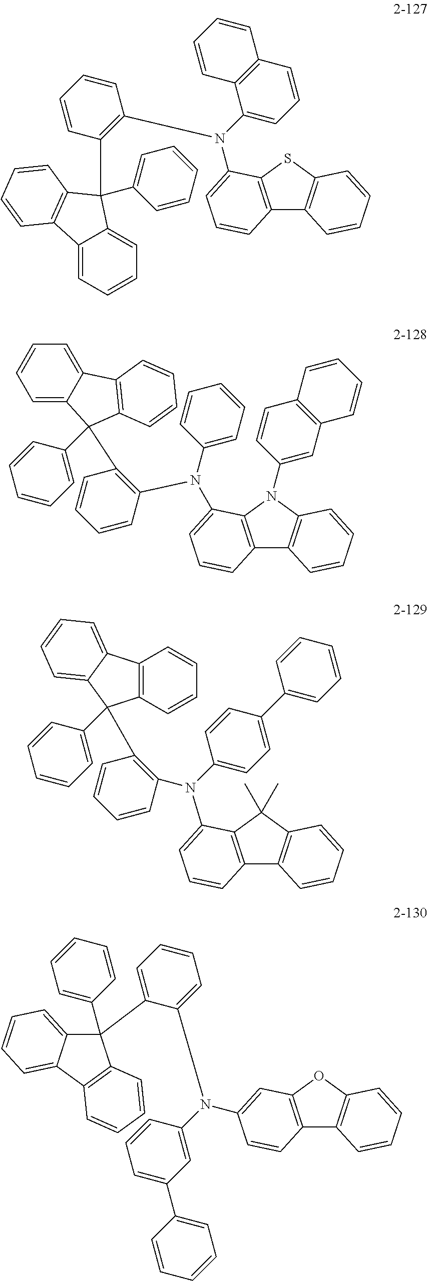 [SMILES: c1ccc(C2(c3ccccc3N(c3cccc4ccccc34)c3cccc4c3sc3ccccc34)c3ccccc3-c3ccccc32)cc1 ; c1ccc(N(c2ccccc2C2(c3ccccc3)c3ccccc3-c3ccccc32)c2cccc3c4ccccc4n(-c4ccc5ccccc5c4)c23)cc1 ; CC1(C)c2ccccc2-c2cccc(N(c3ccc(-c4ccccc4)cc3)c3ccccc3C3(c4ccccc4)c4ccccc4-c4ccccc43)c21 ; c1ccc(-c2cccc(N(c3ccc4c(c3)oc3ccccc34)c3ccccc3C3(c4ccccc4)c4ccccc4-c4ccccc43)c2)cc1]

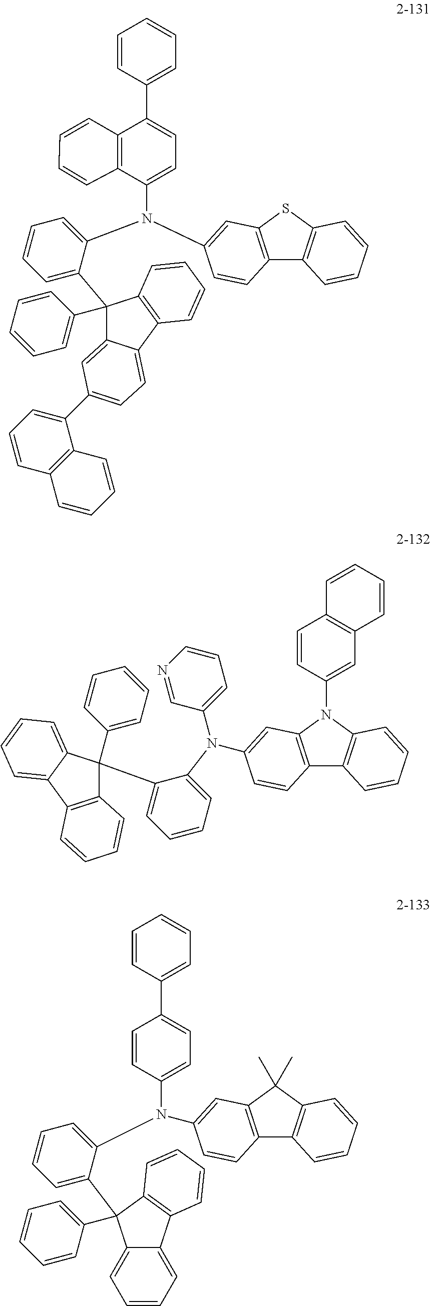 [SMILES: c1ccc(-c2ccc(N(c3ccc4c(c3)sc3ccccc34)c3ccccc3C3(c4ccccc4)c4ccccc4-c4ccc(-c5cccc6ccccc56)cc43)c3ccccc23)cc1 ; c1ccc(C2(c3ccccc3N(c3cccnc3)c3ccc4c5ccccc5n(-c5ccc6ccccc6c5)c4c3)c3ccccc3-c3ccccc32)cc1 ; CC1(C)c2ccccc2-c2ccc(N(c3ccc(-c4ccccc4)cc3)c3ccccc3C3(c4ccccc4)c4ccccc4-c4ccccc43)cc21]

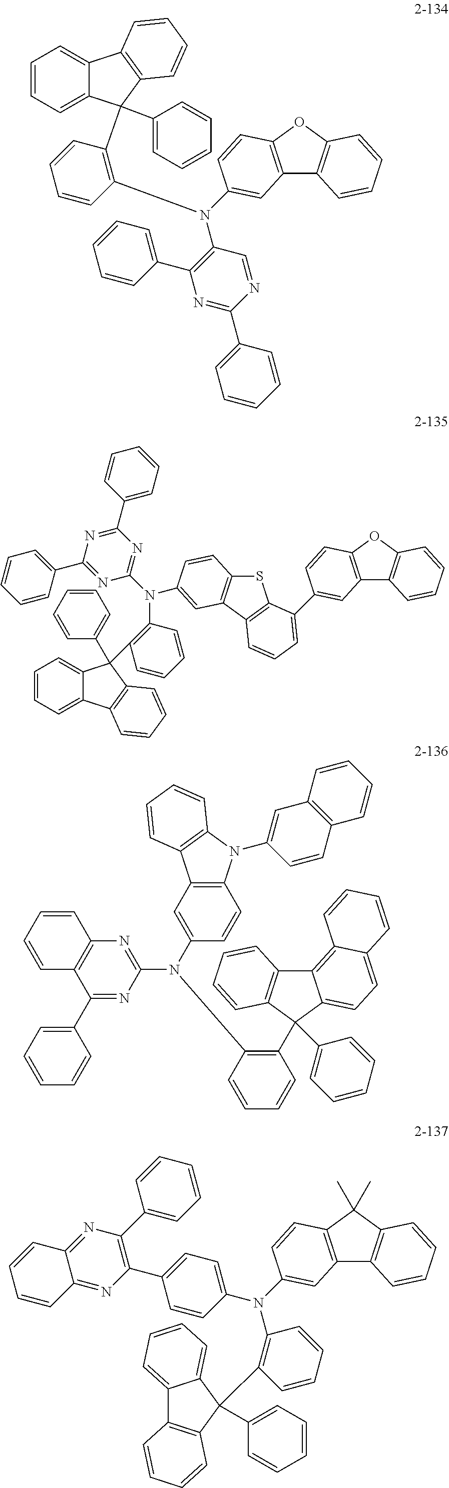 [SMILES: c1ccc(-c2ncc(N(c3ccc4oc5ccccc5c4c3)c3ccccc3C3(c4ccccc4)c4ccccc4-c4ccccc43)c(-c3ccccc3)n2)cc1 ; c1ccc(-c2nc(-c3ccccc3)nc(N(c3ccc4sc5c(-c6ccc7oc8ccccc8c7c6)cccc5c4c3)c3ccccc3C3(c4ccccc4)c4ccccc4-c4ccccc43)n2)cc1 ; c1ccc(-c2nc(N(c3ccc4c(c3)c3ccccc3n4-c3ccc4ccccc4c3)c3ccccc3C3(c4ccccc4)c4ccccc4-c4c3ccc3ccccc43)nc3ccccc23)cc1 ; CC1(C)c2ccccc2-c2cc(N(c3ccc(-c4nc5ccccc5nc4-c4ccccc4)cc3)c3ccccc3C3(c4ccccc4)c4ccccc4-c4ccccc43)ccc21]

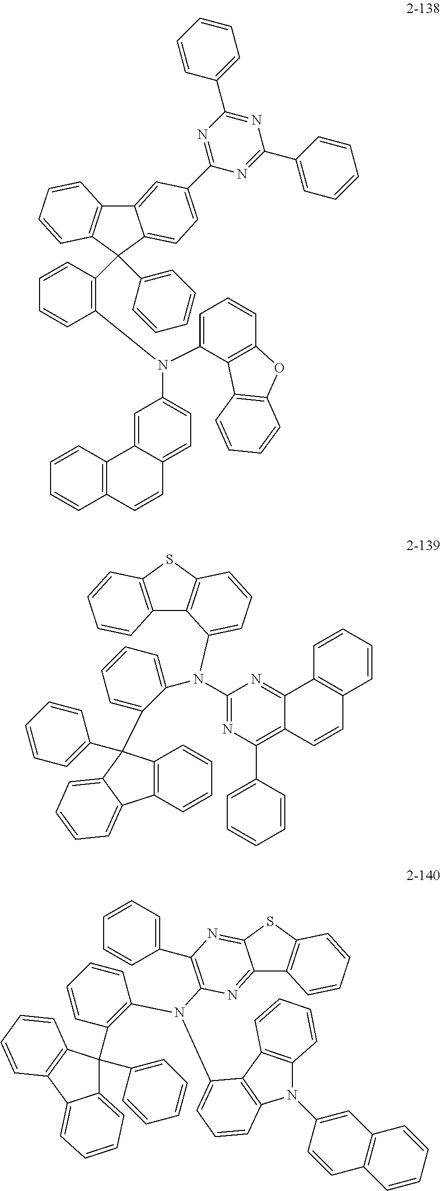 [SMILES: c1ccc(-c2nc(-c3ccccc3)nc(-c3ccc4c(c3)-c3ccccc3C4(c3ccccc3)c3ccccc3N(c3ccc4ccc5ccccc5c4c3)c3cccc4oc5ccccc5c34)n2)cc1 ; c1ccc(-c2nc(N(c3ccccc3C3(c4ccccc4)c4ccccc4-c4ccccc43)c3cccc4sc5ccccc5c34)nc3c2ccc2ccccc23)cc1 ; c1ccc(-c2nc3sc4ccccc4c3nc2N(c2ccccc2C2(c3ccccc3)c3ccccc3-c3ccccc32)c2cccc3c2c2ccccc2n3-c2ccc3ccccc3c2)cc1]

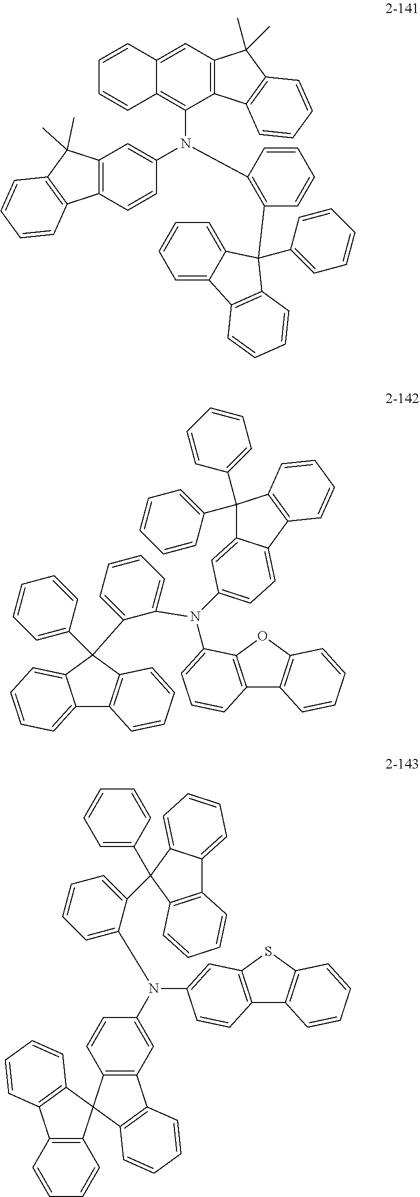 [SMILES: CC1(C)c2ccccc2-c2ccc(N(c3ccccc3C3(c4ccccc4)c4ccccc4-c4ccccc43)c3c4c(cc5ccccc35)C(C)(C)c3ccccc3-4)cc21 ; c1ccc(C2(c3ccccc3)c3ccccc3-c3ccc(N(c4ccccc4C4(c5ccccc5)c5ccccc5-c5ccccc54)c4cccc5c4oc4ccccc45)cc32)cc1 ; c1ccc(C2(c3ccccc3N(c3ccc4c(c3)-c3ccccc3C43c4ccccc4-c4ccccc43)c3ccc4c(c3)sc3ccccc34)c3ccccc3-c3ccccc32)cc1]

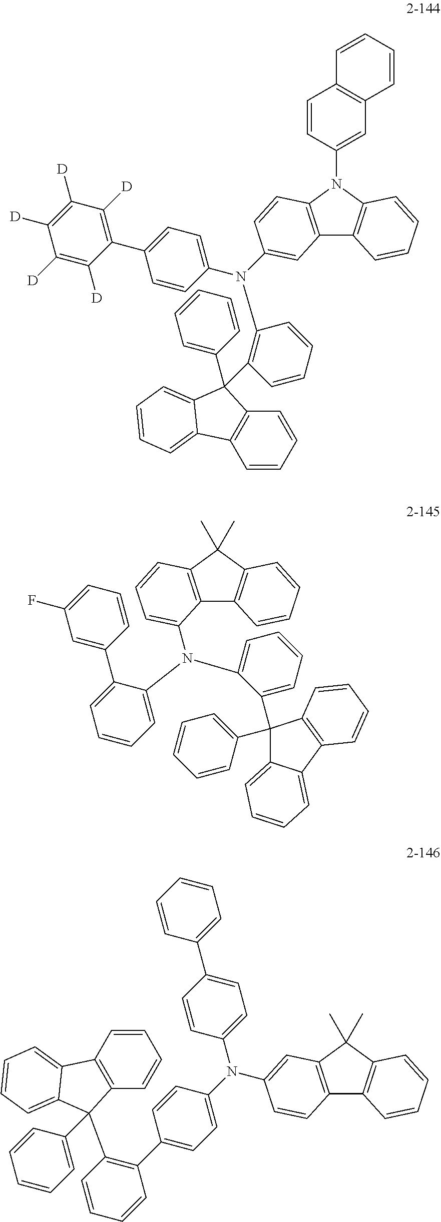 [SMILES: [2H]c1c([2H])c([2H])c(-c2ccc(N(c3ccc4c(c3)c3ccccc3n4-c3ccc4ccccc4c3)c3ccccc3C3(c4ccccc4)c4ccccc4-c4ccccc43)cc2)c([2H])c1[2H] ; CC1(C)c2ccccc2-c2c(N(c3ccccc3-c3cccc(F)c3)c3ccccc3C3(c4ccccc4)c4ccccc4-c4ccccc43)cccc21 ; CC1(C)c2ccccc2-c2ccc(N(c3ccc(-c4ccccc4)cc3)c3ccc(-c4ccccc4C4(c5ccccc5)c5ccccc5-c5ccccc54)cc3)cc21]

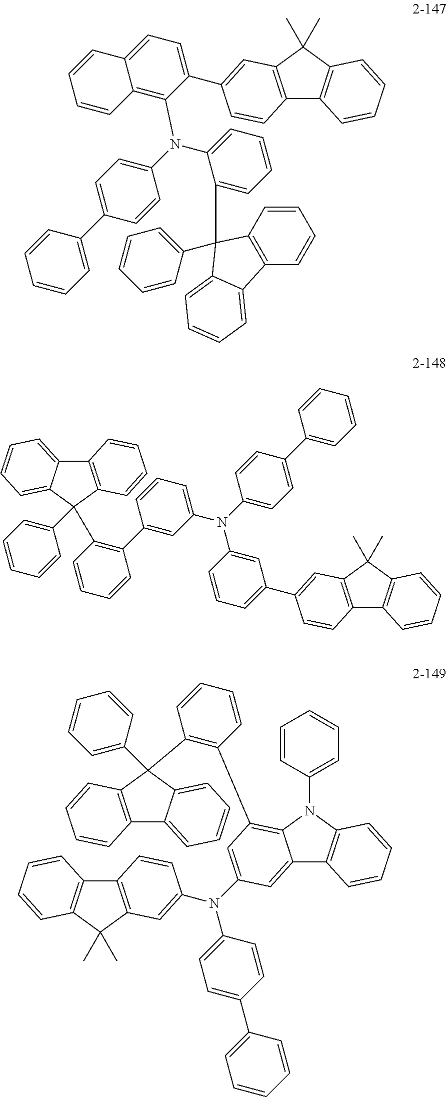 [SMILES: CC1(C)c2ccccc2-c2ccc(-c3ccc4ccccc4c3N(c3ccc(-c4ccccc4)cc3)c3ccccc3C3(c4ccccc4)c4ccccc4-c4ccccc43)cc21 ; CC1(C)c2ccccc2-c2ccc(-c3cccc(N(c4ccc(-c5ccccc5)cc4)c4cccc(-c5ccccc5C5(c6ccccc6)c6ccccc6-c6ccccc65)c4)c3)cc21 ; CC1(C)c2ccccc2-c2ccc(N(c3ccc(-c4ccccc4)cc3)c3cc(-c4ccccc4C4(c5ccccc5)c5ccccc5-c5ccccc54)c4c(c3)c3ccccc3n4-c3ccccc3)cc21]

The present invention provides a compound represented by Formula (37).

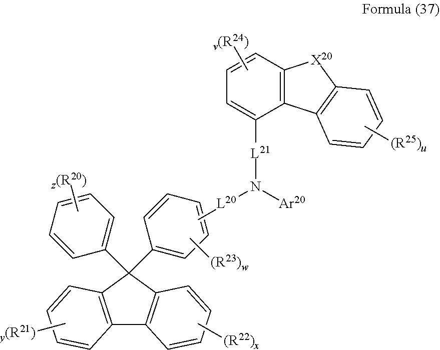

in Formula (37),
1) R²⁰, R²¹, R²², R²³ and R²⁴ are each independently selected from the group consisting of hydrogen; deuterium; halogen; a C₆-C₃₀ aryl group; a fluorenyl group; a C₂-C₅₀ heterocyclic group including at least one heteroatom of O, N, S, Si or P; a fused ring group of a C₃-C₆₀ aliphatic ring and a C₆-C₆₀ aromatic ring; a C₁-C₂₀ alkyl group; a C₂-C₂₀ alkenyl group; a C₂-C₂₀ alkynyl group; a C₁-C₃₀ alkoxyl group; or plurality of R²⁰, plurality of R²¹, plurality of R²², plurality of R²³, and plurality of R²⁴ may be bonded to each other to form an aromatic ring or heteroaromatic ring,
2) R²⁵ is hydrogen; or deuterium; plurality of R²⁵ may be bonded to each other to form an aromatic ring,
3) v is an integer of 0 to 3, u, w, x and y are each independently an integer of 0 to 4, z is an integer of 0 to 5,
4) L²⁰ is a single bond; a C₆-C₅₀ arylene group; or a C₃-C₃₀ heteroarylene group; L²¹ is a single bond;
5) Ar²⁰ is a C₆-C₃₀ aryl group; or a C₃-C₃₀ heteroaryl group;
X²⁰ is O or S,
wherein, the aryl group, arylene group, heterocyclic group, fluorenyl group, heteroaryl group, heteroarylene group, fused ring group, alkyl group, alkenyl group, alkynyl group, and alkoxy group may be substituted with one or more substituents selected from the group consisting of deuterium; halogen; a silane group; siloxane group; boron group; germanium group; cyano group; nitro group; a C₁-C₂₀ alkylthio group; C₁-C₂₀ alkoxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group and C₈-C₂₀ arylalkenyl group, wherein the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, the present invention provides a compound, wherein R²⁴ is a substituent other than H or deuterium.

Also, the compound represented by Formula 37 may be represented by the following Formula 37-1 or Formula 37-3.

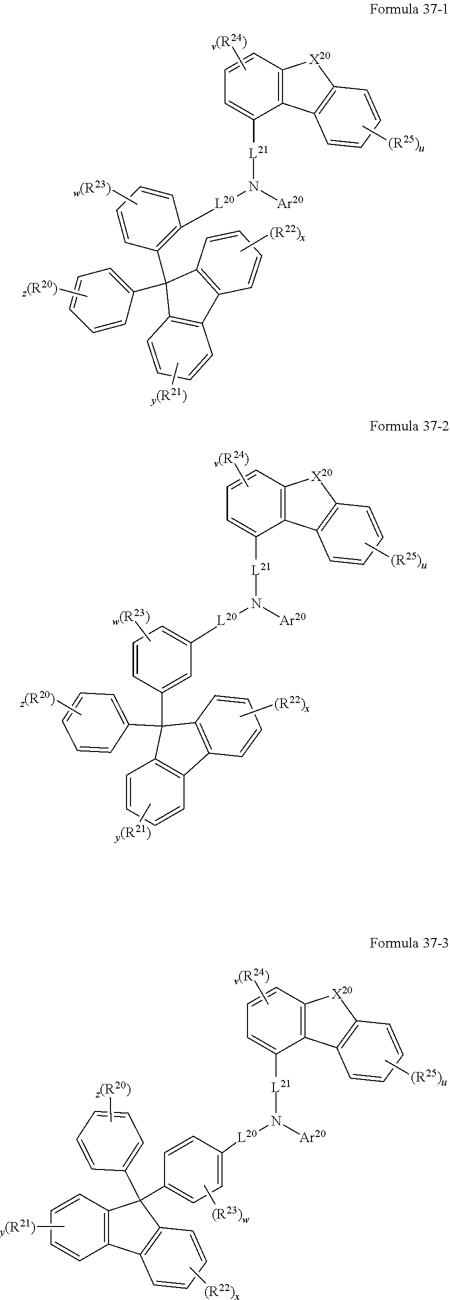

in Formulas (37-1) to (37-3),

R²⁰, R²¹, R²², R²³, R²⁴, R²⁵, L²⁰, L²¹, Ar²⁰ and X²⁰, u, v, w, x, y and z are the same as defined above.

Also, the compound represented by Formula 37 may be represented by the following Formula 37-4.

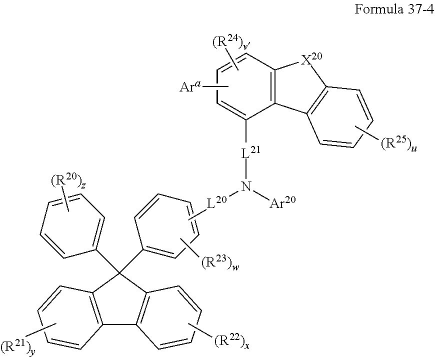

in Formula (37-4),
R²⁰, R²¹, R²², R²³, R²⁴, R²⁵, L²⁰, L²¹, Ar²⁰ and X²⁰, u, w, x, y and z are the same as defined above, v′ is is an integer of 0 to 2,
Ar^{a} is a C₆-C₃₀ aryl group; a C₃-C₅₀ heteroaryl group; or a C₁-C₃₀ alkyl group,

Formula 37 may preferably be the following Formula 37-S.

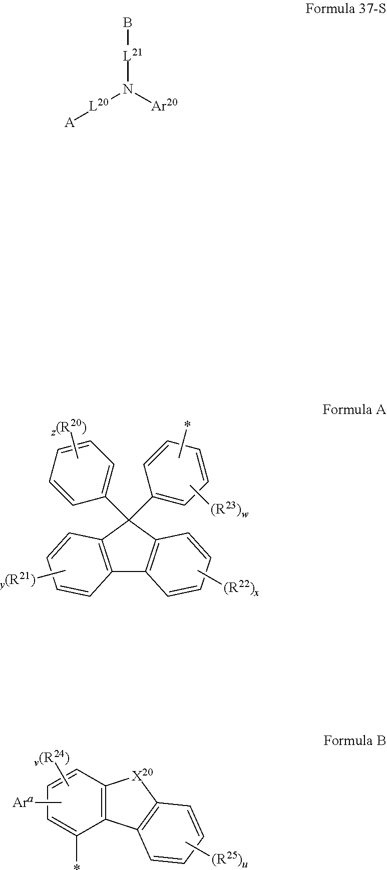

In Formula 37-S,
A is represented by Formula A, and
bonded to L²⁰ of Formula 37-S at *- of Formula A,
and B is represented by Formula B, and
bonded to L²¹ of Formula 37-S at *- of Formula B.

R²⁰, R²¹, R²², R²³, R²⁴, R²⁵, L²⁰, L²¹, Ar²⁰ and X²⁰, u,v, w, x, y and z are the same as defined above.

Formula A may preferably be Formula A-1 to Formula A-3.

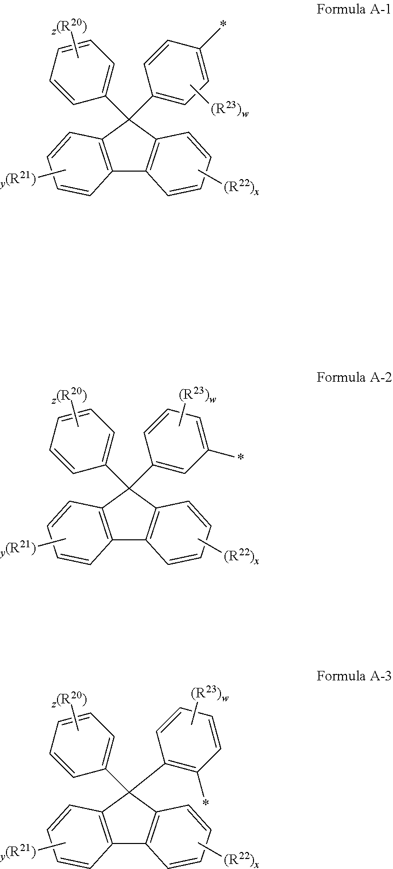

Formula B may preferably be Formula B-1 to Formula B-3.

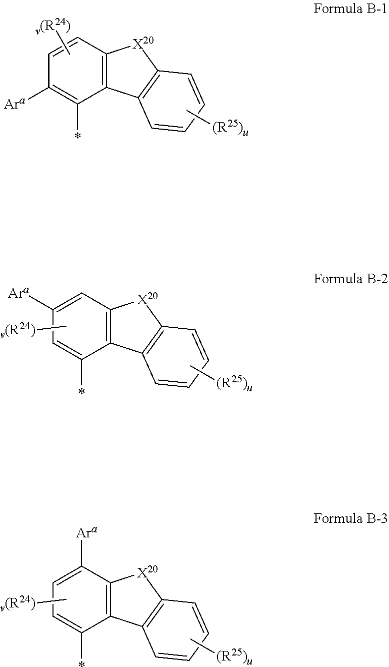

The present invention provides a compound wherein Ar²⁰ is represented by any one of Formulas Ar-1 to Ar-9.

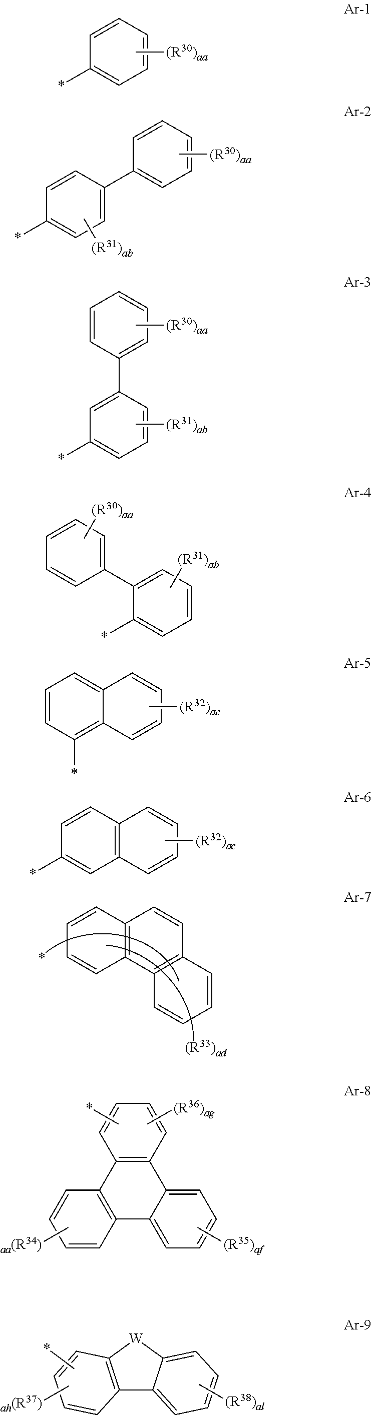

In Formula Ar-1 to Ar-9,
1) R³⁰, R³¹, R³², R³³, R³⁴, R³⁵, R³⁶, R³⁷ and R³⁸ are each independently selected from the group consisting of hydrogen; deuterium; C₁-C₃₀ alkyl group; C₂-C₂₀ alkenyl group; C₂-C₂₀ alkynyl group; a C₁-C₂₀ alkoxy group; a C₆-C₂₀ aryl group; a fluorenyl group; a C₂-C₂₀ heterocyclic group including at least one hetero atom of O, N, S, Si, P; a fused ring group of a C₃-C₆₀ aliphatic ring and a C₆-C₂₀ aromatic ring; and each as plurality are the same as or different from each other, and a plurality of R³⁰ or a plurality of R³¹ or a plurality of R³² or a plurality of R³³ or or a plurality of R³⁴ or a plurality of R³⁵ or a plurality of R³⁶ or a plurality of R³⁷ or a plurality of R³⁸ may be bonded to each other to form an aromatic or a heteroaromatic ring,
2) W is O, S, CR⁴¹R⁴² or NR⁴³,
R⁴¹, R⁴² and R⁴³ are each independently selected from the group consisting of hydrogen; deuterium; C₁-C₃₀ alkyl group; C₂-C₂₀ alkenyl group; C₂-C₂₀ alkynyl group; a C₁-C₂₀ alkoxy group; a C₆-C₂₀ aryl group; a fluorenyl group; a C₂-C₂₀ heterocyclic group including at least one hetero atom of O, N, S, Si, P; and R⁴¹ and R⁴² may be bonded to each other to form a spiro,
3) aa=an integer of 0 to 5; ab is an integer of 0 to 4; ac is an integer of 0 to 7; ad is an integer of 0 to 9; ae, af and ai are each independently an integer of 0 to 4; ag and ah are each independently an integer of 0 to 3.

In the present invention, the compound represented by Formula (37) comprises the following compounds.

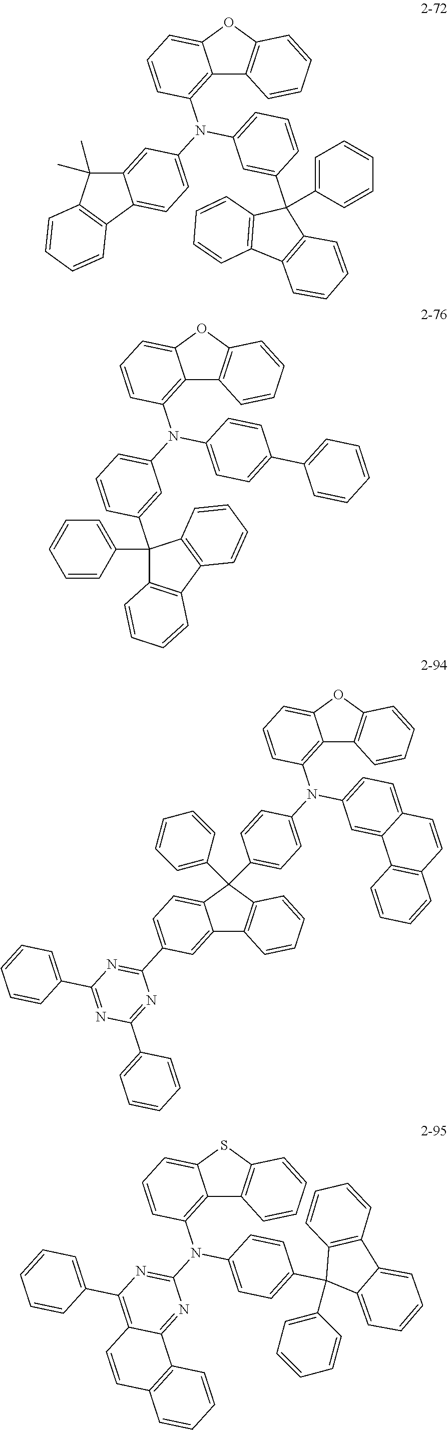 [SMILES: CC1(C)c2ccccc2-c2ccc(N(c3cccc(C4(c5ccccc5)c5ccccc5-c5ccccc54)c3)c3cccc4oc5ccccc5c34)cc21 ; c1ccc(-c2ccc(N(c3cccc(C4(c5ccccc5)c5ccccc5-c5ccccc54)c3)c3cccc4oc5ccccc5c34)cc2)cc1 ; c1ccc(-c2nc(-c3ccccc3)nc(-c3ccc4c(c3)-c3ccccc3C4(c3ccccc3)c3ccc(N(c4ccc5ccc6ccccc6c5c4)c4cccc5oc6ccccc6c45)cc3)n2)cc1 ; c1ccc(-c2nc(N(c3ccc(C4(c5ccccc5)c5ccccc5-c5ccccc54)cc3)c3cccc4sc5ccccc5c34)nc3c2ccc2ccccc23)cc1]

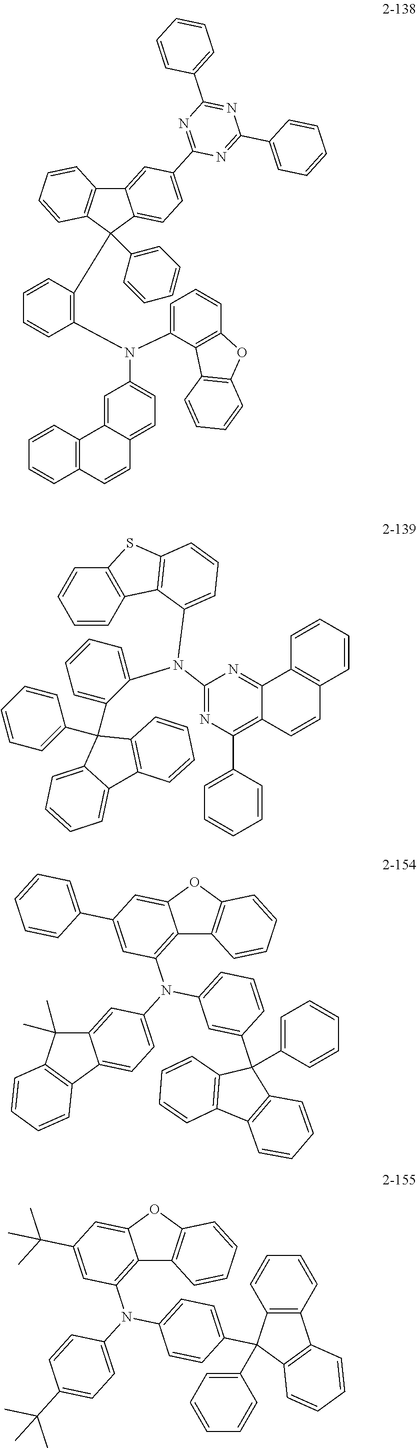 [SMILES: c1ccc(-c2nc(-c3ccccc3)nc(-c3ccc4c(c3)-c3ccccc3C4(c3ccccc3)c3ccccc3N(c3ccc4ccc5ccccc5c4c3)c3cccc4oc5ccccc5c34)n2)cc1 ; c1ccc(-c2nc(N(c3ccccc3C3(c4ccccc4)c4ccccc4-c4ccccc43)c3cccc4sc5ccccc5c34)nc3c2ccc2ccccc23)cc1 ; CC1(C)c2ccccc2-c2ccc(N(c3cccc(C4(c5ccccc5)c5ccccc5-c5ccccc54)c3)c3cc(-c4ccccc4)cc4oc5ccccc5c34)cc21 ; CC(C)(C)c1ccc(N(c2ccc(C3(c4ccccc4)c4ccccc4-c4ccccc43)cc2)c2cc(C(C)(C)C)cc3oc4ccccc4c23)cc1]

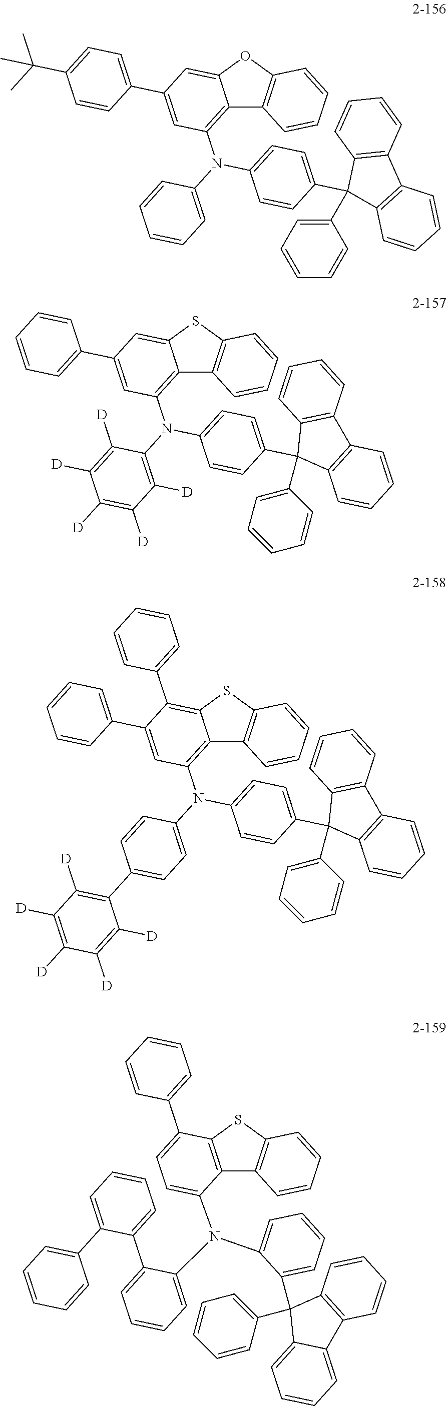 [SMILES: CC(C)(C)c1ccc(-c2cc(N(c3ccccc3)c3ccc(C4(c5ccccc5)c5ccccc5-c5ccccc54)cc3)c3c(c2)oc2ccccc23)cc1 ; [2H]c1c([2H])c([2H])c(N(c2ccc(C3(c4ccccc4)c4ccccc4-c4ccccc43)cc2)c2cc(-c3ccccc3)cc3sc4ccccc4c23)c([2H])c1[2H] ; [2H]c1c([2H])c([2H])c(-c2ccc(N(c3ccc(C4(c5ccccc5)c5ccccc5-c5ccccc54)cc3)c3cc(-c4ccccc4)c(-c4ccccc4)c4sc5ccccc5c34)cc2)c([2H])c1[2H] ; c1ccc(-c2ccccc2-c2ccccc2N(c2ccccc2C2(c3ccccc3)c3ccccc3-c3ccccc32)c2ccc(-c3ccccc3)c3sc4ccccc4c23)cc1]

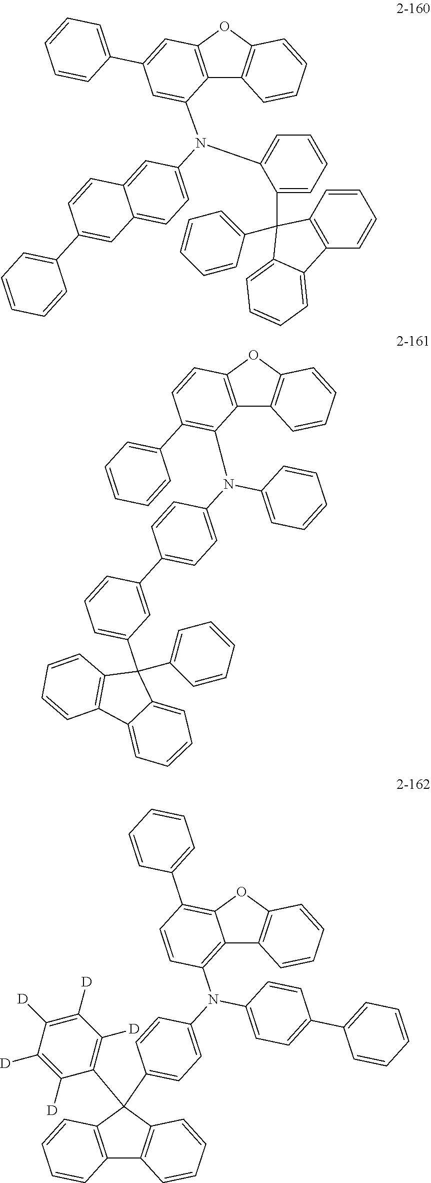

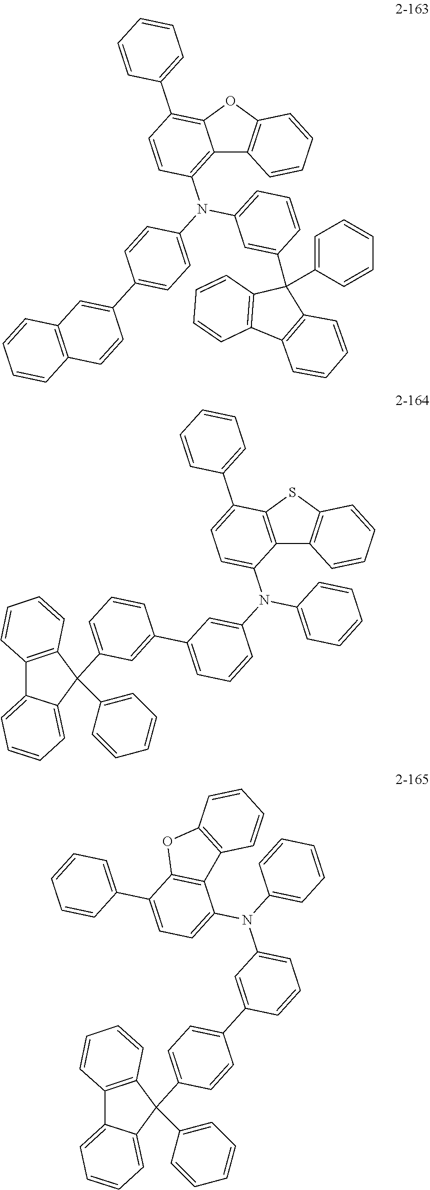 [SMILES: c1ccc(-c2ccc(N(c3ccc(-c4ccc5ccccc5c4)cc3)c3cccc(C4(c5ccccc5)c5ccccc5-c5ccccc54)c3)c3c2oc2ccccc23)cc1 ; c1ccc(-c2ccc(N(c3ccccc3)c3cccc(-c4cccc(C5(c6ccccc6)c6ccccc6-c6ccccc65)c4)c3)c3c2sc2ccccc23)cc1 ; c1ccc(-c2ccc(N(c3ccccc3)c3cccc(-c4ccc(C5(c6ccccc6)c6ccccc6-c6ccccc65)cc4)c3)c3c2oc2ccccc23)cc1]

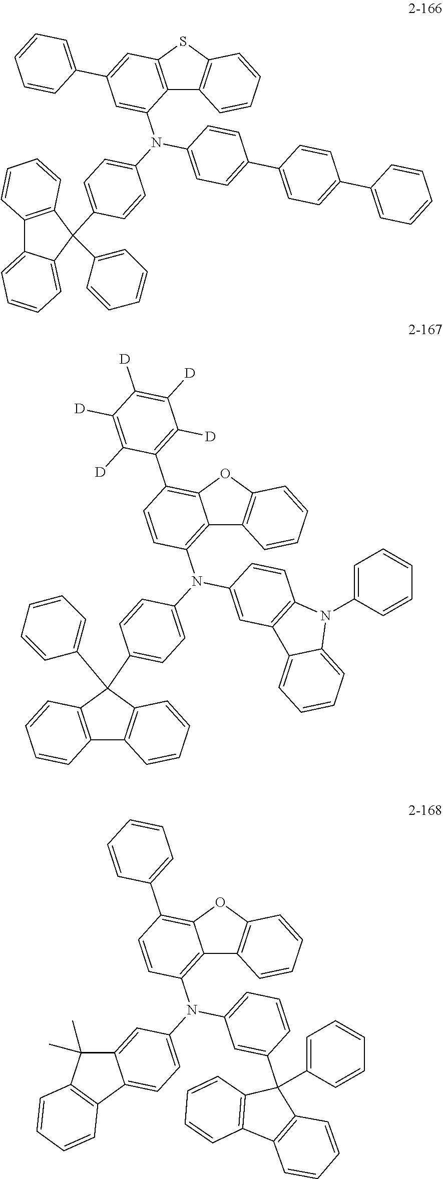 [SMILES: c1ccc(-c2ccc(-c3ccc(N(c4ccc(C5(c6ccccc6)c6ccccc6-c6ccccc65)cc4)c4cc(-c5ccccc5)cc5sc6ccccc6c45)cc3)cc2)cc1 ; [2H]c1c([2H])c([2H])c(-c2ccc(N(c3ccc(C4(c5ccccc5)c5ccccc5-c5ccccc54)cc3)c3ccc4c(c3)c3ccccc3n4-c3ccccc3)c3c2oc2ccccc23)c([2H])c1[2H] ; CC1(C)c2ccccc2-c2ccc(N(c3cccc(C4(c5ccccc5)c5ccccc5-c5ccccc54)c3)c3ccc(-c4ccccc4)c4oc5ccccc5c34)cc21]

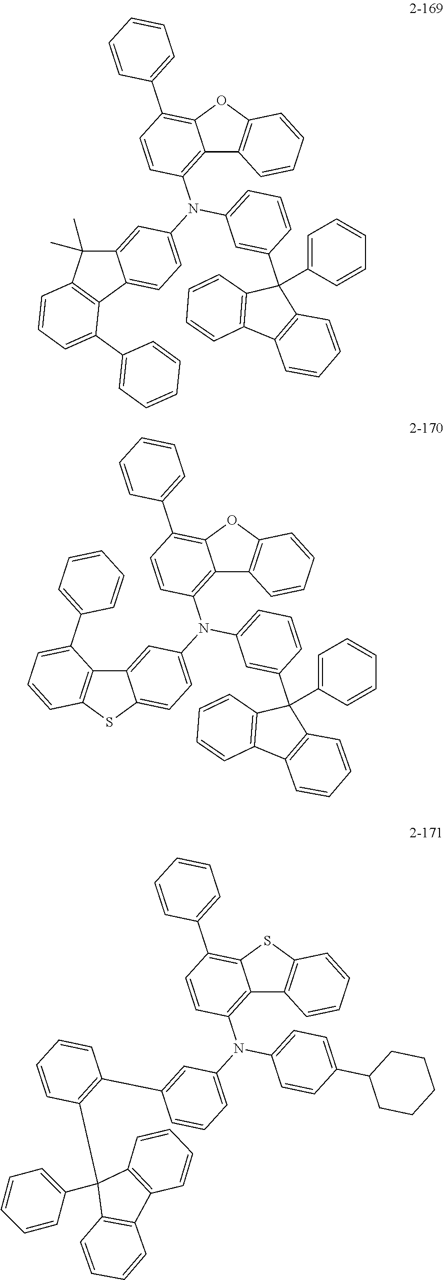

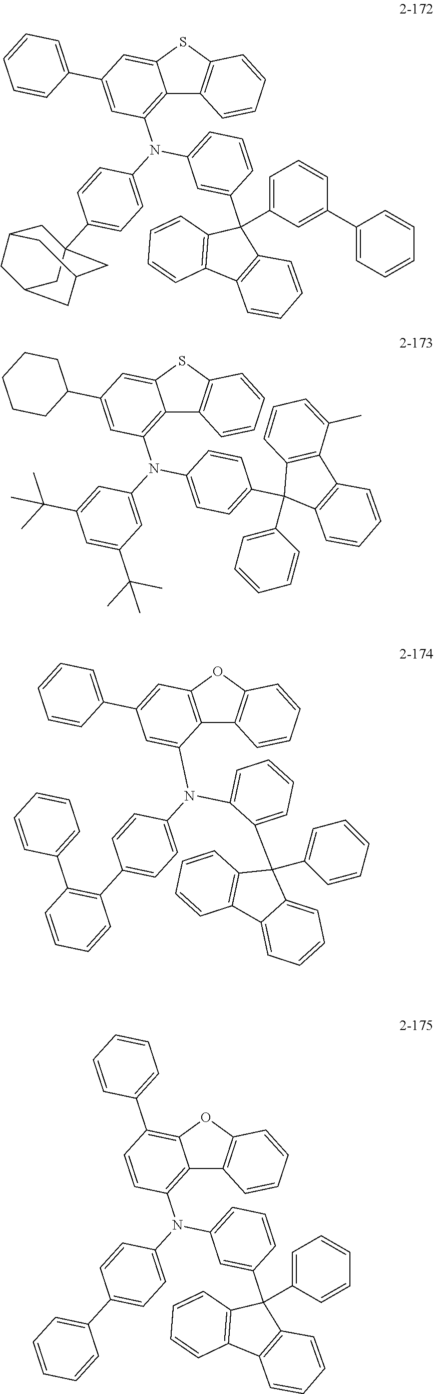 [SMILES: c1ccc(-c2cccc(C3(c4cccc(N(c5ccc(C67CC8CC(CC(C8)C6)C7)cc5)c5cc(-c6ccccc6)cc6sc7ccccc7c56)c4)c4ccccc4-c4ccccc43)c2)cc1 ; Cc1cccc2c1-c1ccccc1C2(c1ccccc1)c1ccc(N(c2cc(C(C)(C)C)cc(C(C)(C)C)c2)c2cc(C3CCCCC3)cc3sc4ccccc4c23)cc1 ; c1ccc(-c2cc(N(c3ccc(-c4ccccc4-c4ccccc4)cc3)c3ccccc3C3(c4ccccc4)c4ccccc4-c4ccccc43)c3c(c2)oc2ccccc23)cc1 ; c1ccc(-c2ccc(N(c3cccc(C4(c5ccccc5)c5ccccc5-c5ccccc54)c3)c3ccc(-c4ccccc4)c4oc5ccccc5c34)cc2)cc1]

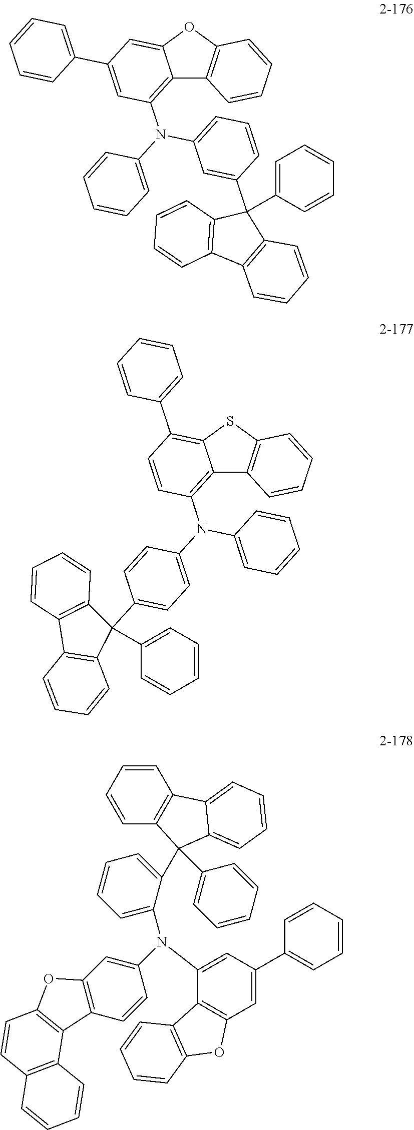 [SMILES: c1ccc(-c2cc(N(c3ccccc3)c3cccc(C4(c5ccccc5)c5ccccc5-c5ccccc54)c3)c3c(c2)oc2ccccc23)cc1 ; c1ccc(-c2ccc(N(c3ccccc3)c3ccc(C4(c5ccccc5)c5ccccc5-c5ccccc54)cc3)c3c2sc2ccccc23)cc1 ; c1ccc(-c2cc(N(c3ccc4c(c3)oc3ccc5ccccc5c34)c3ccccc3C3(c4ccccc4)c4ccccc4-c4ccccc43)c3c(c2)oc2ccccc23)cc1]

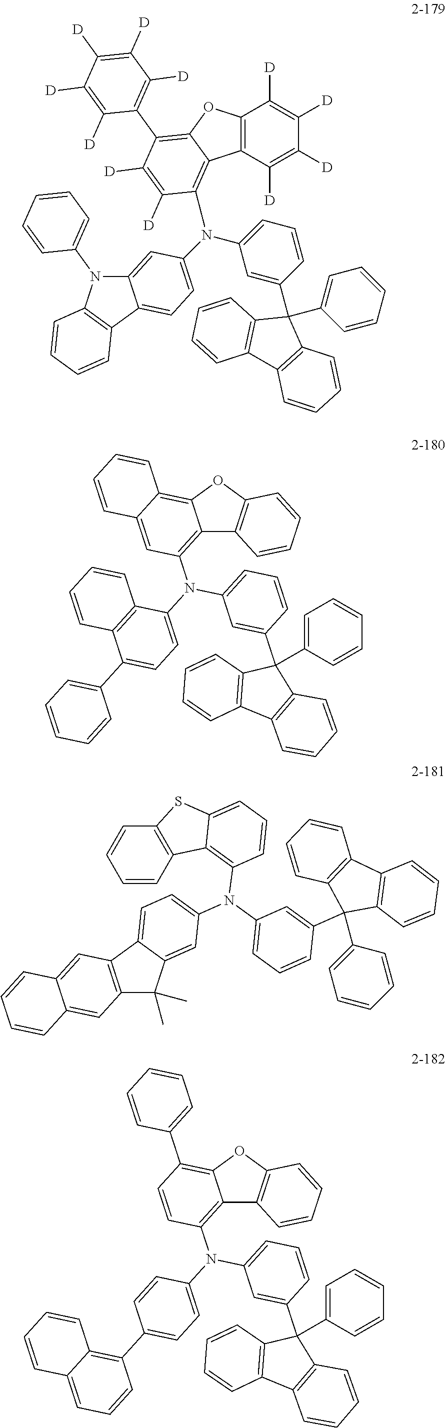

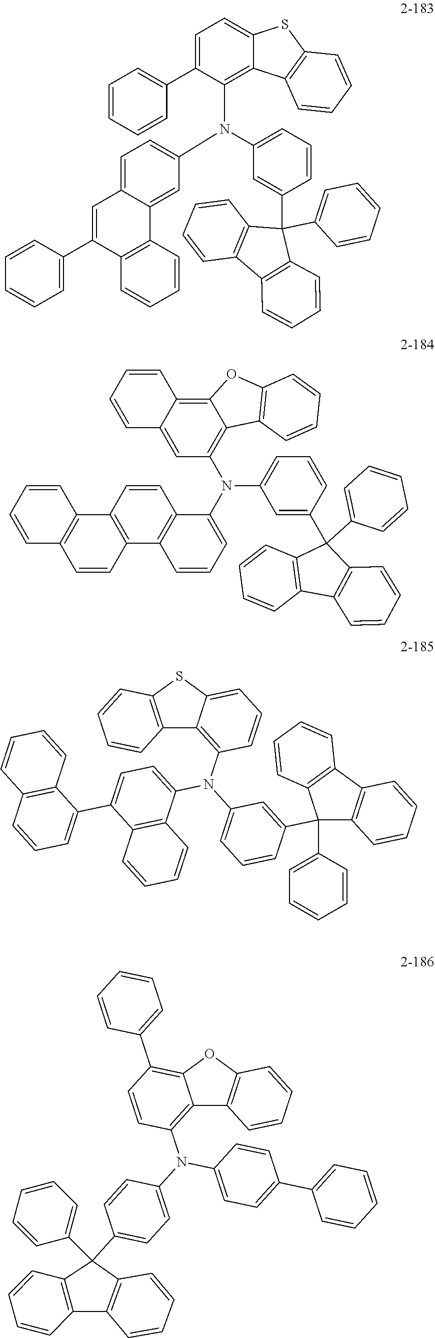

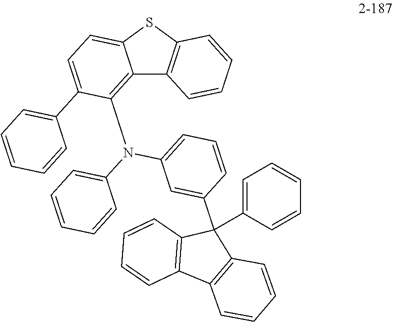 [SMILES: c1ccc(-c2ccc3sc4ccccc4c3c2N(c2ccccc2)c2cccc(C3(c4ccccc4)c4ccccc4-c4ccccc43)c2)cc1]

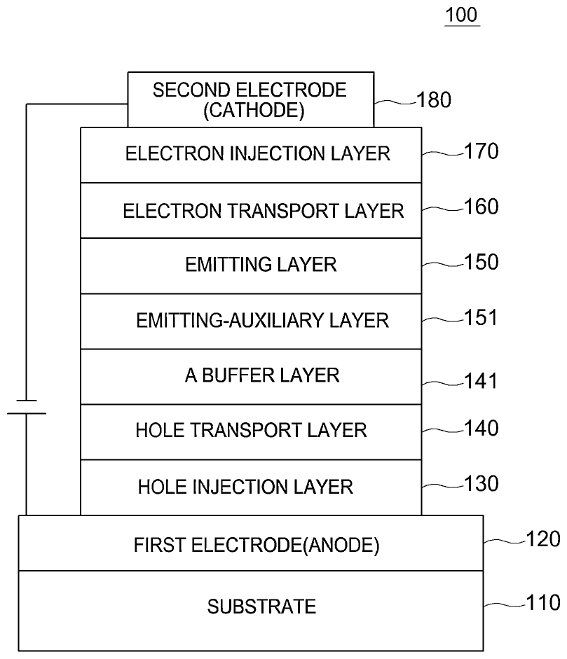

Referring to FIG. 1, the organic electric element (100) according to the present invention includes a first electrode (120) formed on a substrate (110), a second electrode (180), and an organic material layer including the compound represented by Formula (1) between the first electrode (120) and the second electrode (180). Here, the first electrode (120) may be an anode (positive electrode), and the second electrode (180) may be a cathode (negative electrode). In the case of an inverted organic electric element, the first electrode may be a cathode, and the second electrode may be an anode.

The organic material layer may include a hole injection layer (130), a hole transport layer (140), an emitting layer (150), an emitting auxiliary layer (151), an electron transport layer (160), and an electron injection layer (170) formed in sequence on the first electrode (120). Here, the remaining layers except the emitting layer (150) may not be formed. The organic material layer may further include a hole blocking layer, an electron blocking layer, an emitting-auxiliary layer (151), an electron transport auxiliary layer, a buffer layer (141), etc., and the electron transport layer (160) and the like may serve as a hole blocking layer.

Although not shown, the organic electric element according to the present invention may further include a protective layer formed on at least one side of the first and second electrodes, which is a side opposite to the organic material layer.

Otherwise, even if the same core is used, the band gap, the electrical characteristics, the interface characteristics, and the like may vary depending on which substituent is bonded at which position, therefore the choice of core and the combination of sub-substituents associated therewith is also very important, and in particular, when the optimal combination of energy levels and T1 values and unique properties of materials (mobility, interfacial characteristics, etc.) of each organic material layer is achieved, a long life span and high efficiency can be achieved at the same time.

The organic electroluminescent device according to an embodiment of the present invention may be manufactured using a PVD (physical vapor deposition) method. For example, a metal or a metal oxide having conductivity or an alloy thereof is deposited on a substrate to form a cathode, and the organic material layer including the hole injection layer (130), the hole transport layer (140), the emitting layer (150), the electron transport layer (160), and the electron injection layer (170) is formed thereon, and then depositing a material usable as a cathode thereon can manufacture an organic electroluminescent device according to an embodiment of the present invention. In addition, an emission auxiliary layer (151) may be further formed between the hole transport layer (140) and the emitting layer (150), and an electron transport auxiliary layer may be further formed between the emitting layer (150) and the electron transport layer (160).

As another specific example, the present invention provides an organic electric element wherein the emitting layer in the organic material layer is a phosphorescent light emitting layer.

The present invention may further include a light efficiency enhancing layer formed on at least one of the opposite side to the organic material layer among one side of the first electrode, or one of the opposite side to the organic material layer among one side of the second electrode.

As another example, the present invention provides an organic electronic device wherein the compound represented by Formula (30) is used in an emitting auxiliary layer or an electron blocking layer and is preferably included in a green emitting auxiliary layer. More specifically, the compound represented by Formula (39) or Formula (40) is included in the green emitting auxiliary layer.

Also, the present invention provides the organic electric element wherein the organic material layer is formed by one of a spin coating process, a nozzle printing process, an inkjet printing process, a slot coating process, a dip coating process or a roll-to-roll process, and since the organic material layer according to the present invention can be formed by various methods, the scope of the present invention is not limited by the method of forming the organic material layer.

The organic electric element according to an embodiment of the present invention may be a front emission type, a back emission type, or a both-sided emission type, depending on the material used.

WOLED (White Organic Light Emitting Device) has advantages of high resolution realization and excellent fairness, and can be manufactured using conventional LCD color filter technology. Various structures for a white organic light emitting device mainly used as a backlight device have been proposed and patented. Representatively, there are side-by-side arrangement of the radiation part of the R(red), G(green) and B(blue), a stacking method in which R, G, and B emitting layers are laminated on top and bottom, electroluminescence by the blue (B) organic emitting layer and, by using the light from this, a color conversion material (CCM) method using a photo-luminescence of an inorganic phosphor, etc., and the present invention may be applied to such WOLED.

The present invention also provides an electronic device comprising a display device including the organic electric element; and a control unit for driving the display device.

According to another aspect, the present invention provides an display device wherein the organic electric element is at least one of an OLED, an organic solar cell, an organic photo conductor, an organic transistor (organic TFT) and an element for monochromic or white illumination. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint(PMP), a remote controller, a navigation unit, a game player, various kinds of TVs, and various kinds of computers.

Hereinafter, Synthesis Examples of the compound represented by Formula (18) of the present invention and preparation examples of the organic electric element of the present invention will be described in detail by way of example, but are not limited to the following examples.

### EXAMPLES

### Synthesis Example 1

Final products represented by Formula (18) according to the present invention can be prepared by reacting as follows, but are not limited thereto.

### Synthesis Example of 13-17

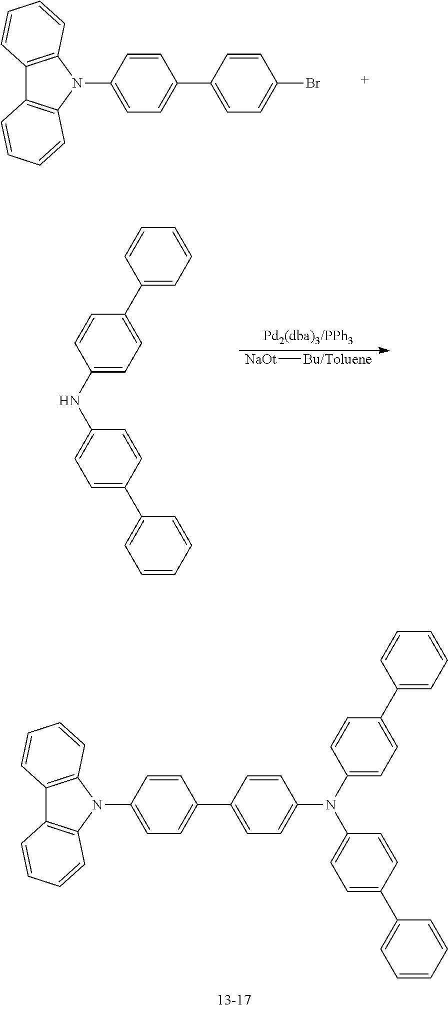

9-(4′-bromo-[1,1′-biphenyl]-4-yl)-9H-carbazole (9.6 g, 24 mmol) was dissolved in toluene, and di([1,1′-biphenyl]-4-yl) amine(6.4 g, 20 mmol), Pd₂(dba)₃ (0.05 eq.), PPh₃(0.1 eq.), NaOt-Bu (3 eq.) were added and refluxed with stirring at 100° C. at 24 hours. After the reaction was completed, the reaction mixture was extracted with ether and water. The organic layer was dried over MgSO₄ and concentrated. The resulting organic material was separated by silicagel column chromatography and recrystallization to obtain 12.9 g (yield: 84%) of the product.

### Synthesis Example of 13-32

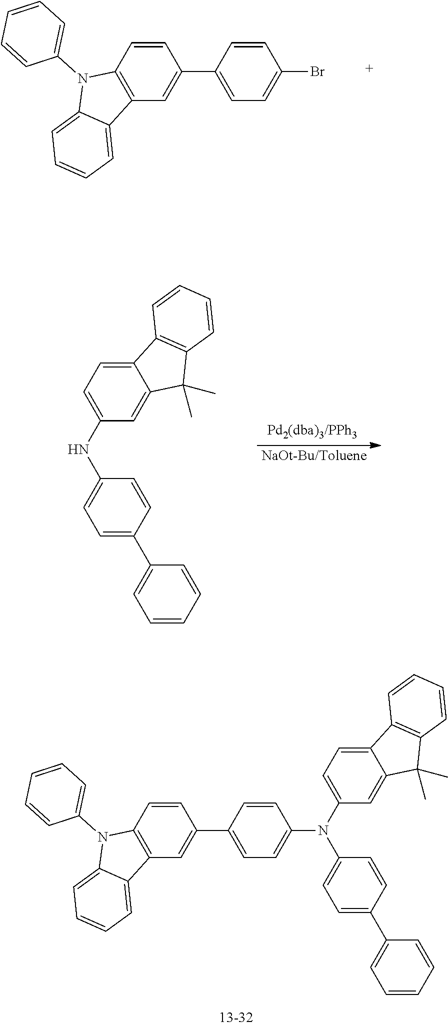 [SMILES: Brc1ccc(-c2ccc3c(c2)c2ccccc2n3-c2ccccc2)cc1 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

3-(4-bromophenyl)-9-phenyl-9H-carbazole (9.6 g, 24 mmol) was dissolved in toluene, and N-([1,1′-biphenyl]-4-yl)-9,9-dimethyl-9H-fluoren-2-amine (7.2 g, 20 mmol), Pd₂(dba)₃ (0.05 eq.), PPh₃(0.1 eq.), NaOt-Bu (3 eq.) were added and refluxed with stirring at 100° C. at 24 hours. After the reaction was completed, the reaction mixture was extracted with ether and water. The organic layer was dried over MgSO₄ and concentrated. The resulting organic material was separated by silicagel column chromatography and recrystallization to obtain 13.8 g (yield: 85%) of the product.

### Synthesis Example of 2-34

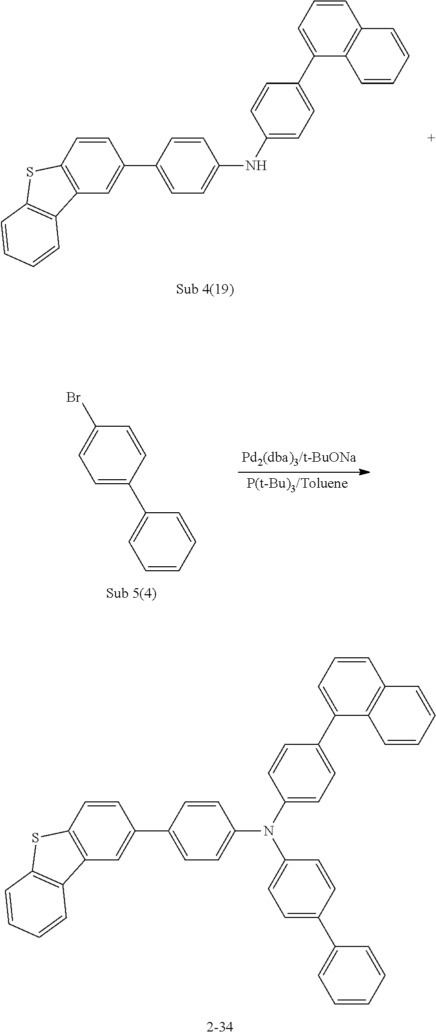 [SMILES: c1ccc2c(-c3ccc(Nc4ccc(-c5ccc6sc7ccccc7c6c5)cc4)cc3)cccc2c1 ; Brc1ccc(-c2ccccc2)cc1 ; c1ccc(-c2ccc(N(c3ccc(-c4ccc5sc6ccccc6c5c4)cc3)c3ccc(-c4cccc5ccccc45)cc3)cc2)cc1]

In a round bottom flask, Sub 4(19) (9.5 g, 20 mmol), Sub 5(4) (4.7 g, 20 mmol), Pd₂(dba)₃ (0.5 g, 0.6 mmol), P(t-Bu)₃ (0.2 g, 2 mmol), t-BuONa (5.8 g, 60 mmol), toluene (300 mL) were added and were carried out at 100° C. When the reaction was complete, the reaction mixture was extracted with CH₂Cl₂ and water. The organic layer was dried over MgSO₄ and concentrated. The resulting compound was separated by silicagel column chromatography and recrystallization to obtain 9.8 g (yield: 78%) of 2-34.

### Synthesis Example of 2-58

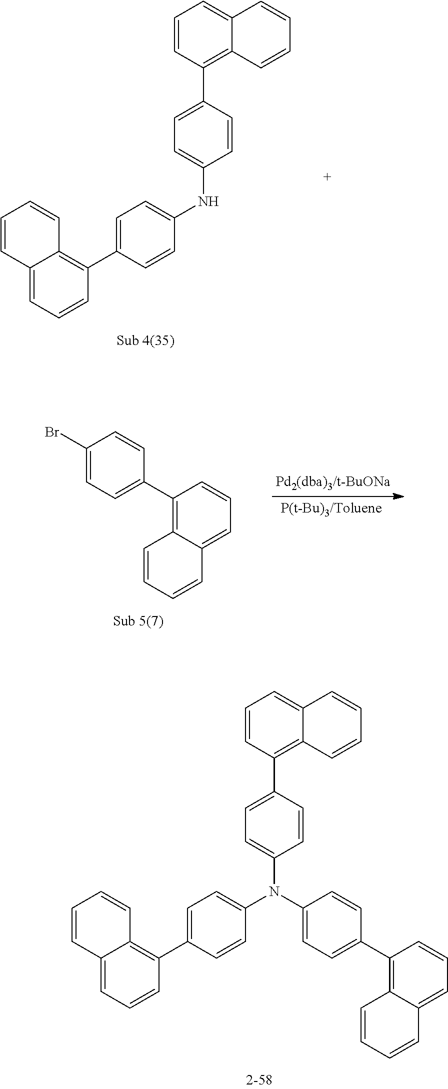 [SMILES: c1ccc2c(-c3ccc(Nc4ccc(-c5cccc6ccccc56)cc4)cc3)cccc2c1 ; Brc1ccc(-c2cccc3ccccc23)cc1 ; c1ccc2c(-c3ccc(N(c4ccc(-c5cccc6ccccc56)cc4)c4ccc(-c5cccc6ccccc56)cc4)cc3)cccc2c1]

In a round bottom flask, Sub 4(35) (8.4 g, 20 mmol), Sub 5(7) (5.7 g, 20 mmol), Pd₂(dba)₃ (0.5 g, 0.6 mmol), P(t-Bu)₃ (0.2 g, 2 mmol), t-BuONa (5.8 g, 60 mmol) and toluene(300 mL) were carried out in the same manner as in 2-34 to give 2-58.(10.4 g, 83%).

### Synthesis Example of 2-59

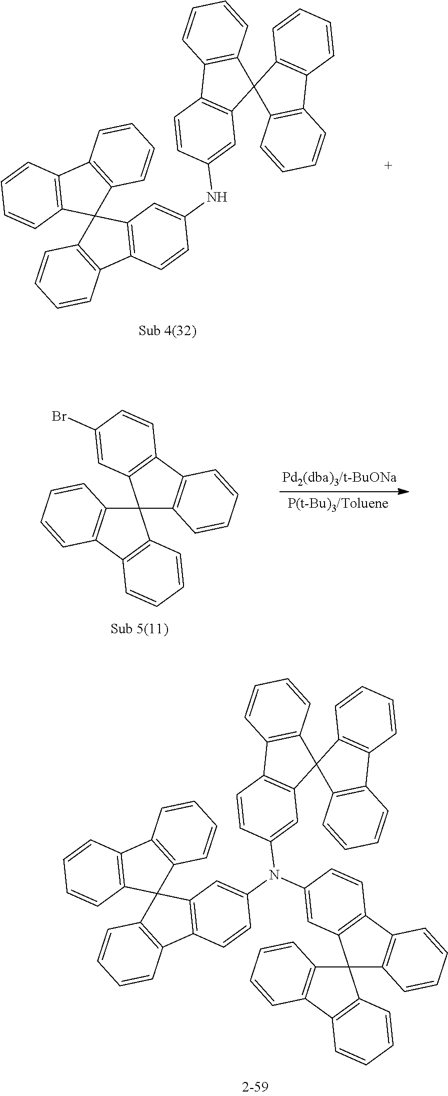 [SMILES: c1ccc2c(c1)-c1ccccc1C21c2ccccc2-c2ccc(Nc3ccc4c(c3)C3(c5ccccc5-c5ccccc53)c3ccccc3-4)cc21 ; Brc1ccc2c(c1)C1(c3ccccc3-c3ccccc31)c1ccccc1-2 ; c1ccc2c(c1)-c1ccccc1C21c2ccccc2-c2ccc(N(c3ccc4c(c3)C3(c5ccccc5-c5ccccc53)c3ccccc3-4)c3ccc4c(c3)C3(c5ccccc5-c5ccccc53)c3ccccc3-4)cc21]

In a round bottom flask, Sub 4(32) (12.9 g, 20 mmol), Sub 5(11) (7.9 g, 20 mmol), Pd₂(dba)₃ (0.5 g, 0.6 mmol), P(t-Bu)₃ (0.2 g, 2 mmol), t-BuONa (5.8 g, 60 mmol) and toluene(300 mL) were carried out in the same manner as in 2-34 to give 2-59. (5.2 g, 79%).

### Synthesis Example of 2-69

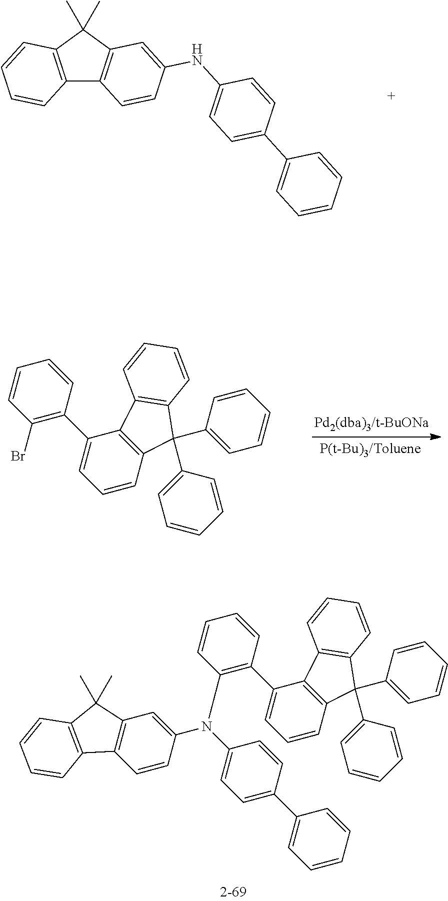 [SMILES: CC1(C)c2ccccc2-c2ccc(Nc3ccc(-c4ccccc4)cc3)cc21 ; Brc1ccccc1-c1cccc2c1-c1ccccc1C2(c1ccccc1)c1ccccc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

In a round bottom flask, N-([1,1′-biphenyl]-4-yl)-9,9-dimethyl-9H-fluoren-2-amine(7.2 g, 20 mmol), 4-(2-bromophenyl)-9,9-diphenyl-9H-fluorene (9.5 g, 20 mmol), Pd₂(dba)₃ (0.5 g, 0.6 mmol), P(t-Bu)₃ (0.2 g, 2 mmol), t-BuONa (5.8 g, 60 mmol) and toluene(300 mL) were carried out in the same manner as in 2-34 to give 2-69. (12.2 g, 81%).

### Synthesis Example of 2-71

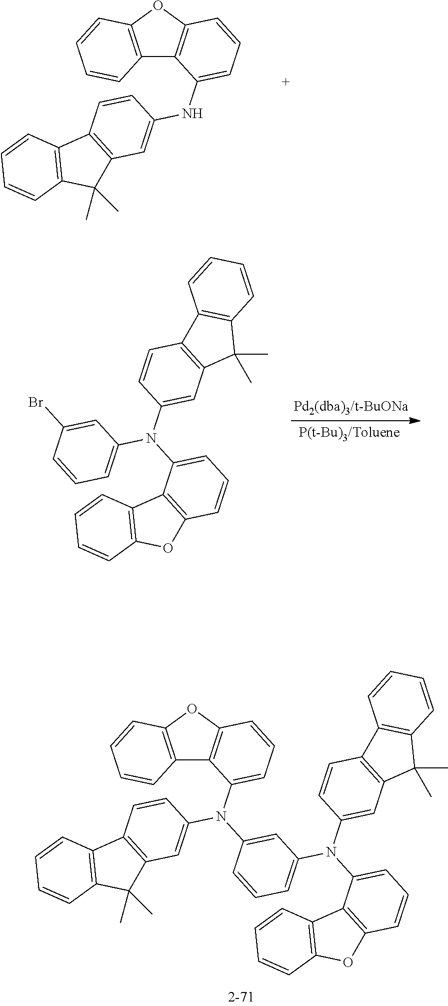 [SMILES: CC1(C)c2ccccc2-c2ccc(Nc3cccc4oc5ccccc5c34)cc21 ; CC1(C)c2ccccc2-c2ccc(N(c3cccc(Br)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3cccc4oc5ccccc5c34)cc21]

In a round bottom flask, N-(9,9-dimethyl-9H-fluoren-2-yl) dibenzo[b,d]furan-1-amine(7.5 g, 20 mmol), N-(3-bromophenyl)-N-(9,9-dimethyl-9H-fluoren-2-yl)dibenzo[b,d]furan-1-amine(10.6 g, 20 mmol), Pd₂(dba)₃(0.5 g, 0.6 mmol), P(t-Bu)₃ (0.2 g, 2 mmol), t-BuONa (5.8 g, 60 mmol) and toluene(300 mL) were carried out in the same manner as in 2-34 to give 2-71. (12.9 g, 78%).

### Synthesis Example 2

Final products represented by Formula (30) according to the present invention can be prepared by reacting Sub 30 and Sub 31 as shown in Reaction Scheme 5 below, but are not limited thereto.

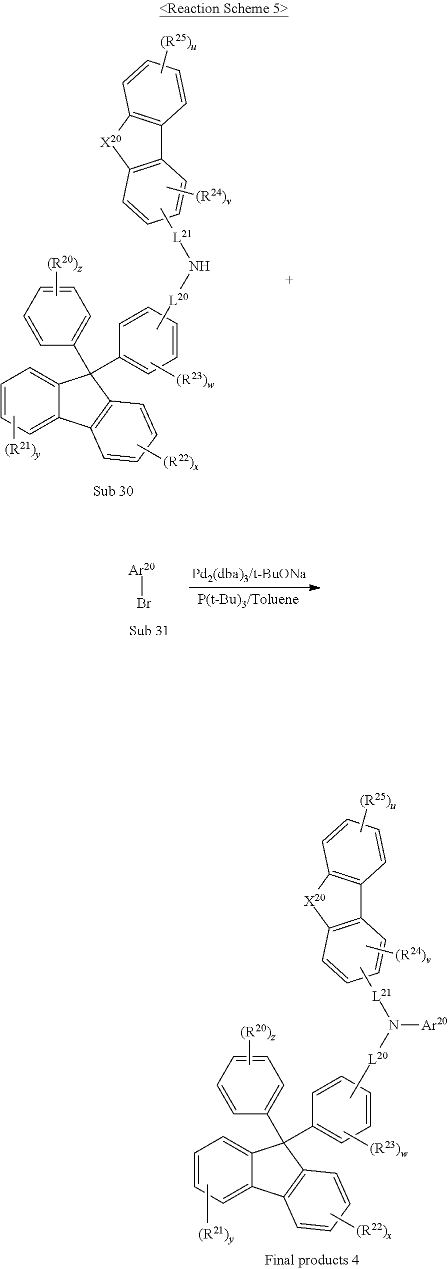

### Synthesis Example of Sub 30

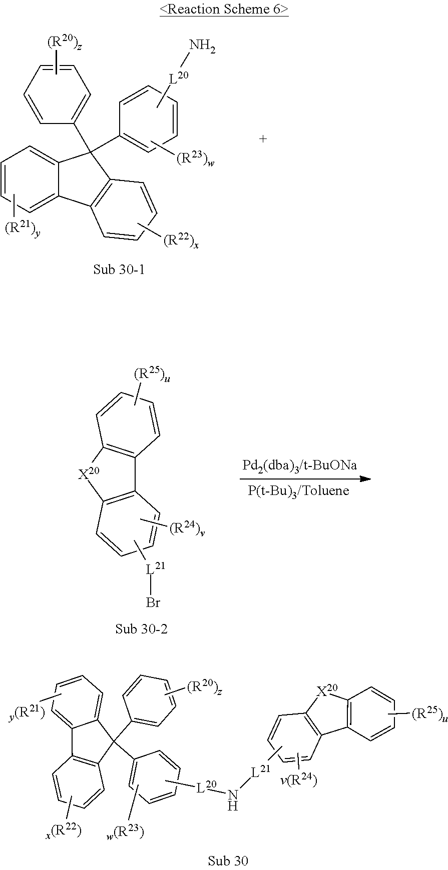

### Synthesis Example of Sub 30(81)

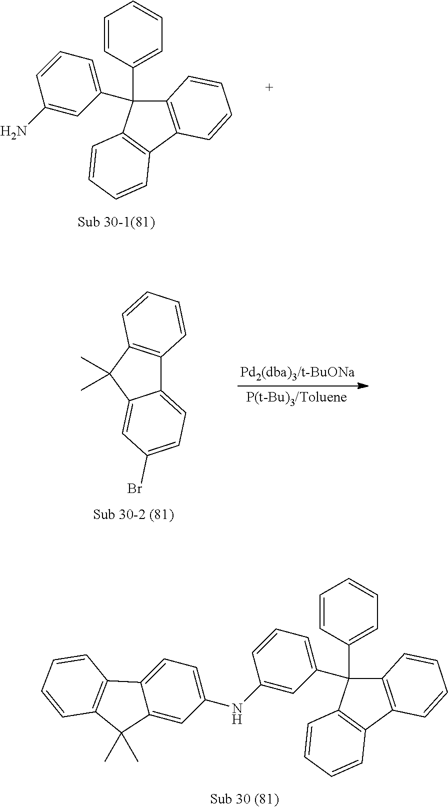 [SMILES: Nc1cccc(C2(c3ccccc3)c3ccccc3-c3ccccc32)c1 ; CC1(C)c2ccccc2-c2ccc(Br)cc21 ; CC1(C)c2ccccc2-c2ccc(Nc3cccc(C4(c5ccccc5)c5ccccc5-c5ccccc54)c3)cc21]

In a round bottom flask, 3-(9-phenyl-9H-fluoren-9-yl)aniline (6.7 g, 20 mmol), 2-bromo-9,9-dimethyl-9H-fluorene (5.5 g, 20 mmol), Pd₂(dba)₃ (0.5 g, 0.6 mmol), P(t-Bu)₃ (0.2 g, 2 mmol), t-BuONa (5.8 g, 60 mmol) and toluene(300 mL) were carried out in the same manner as in 2-34 to give Sub 30(81). (8.83 g, 84%).

### Examples of Sub 31

Examples of Sub 31 are as follows, but are not limited thereto.

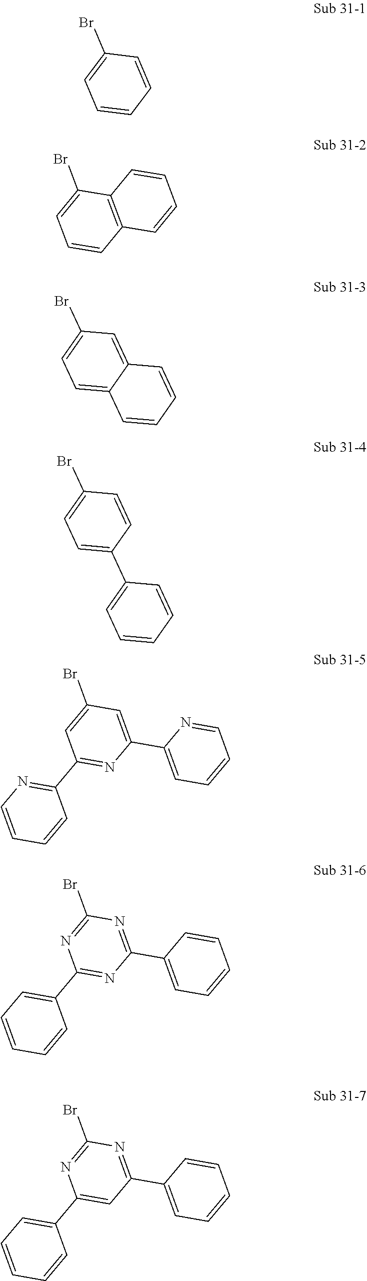

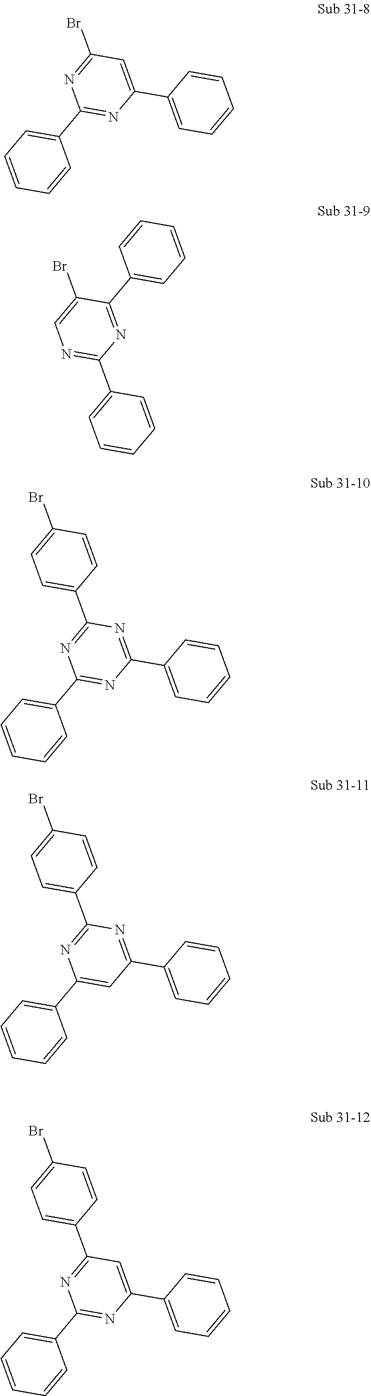

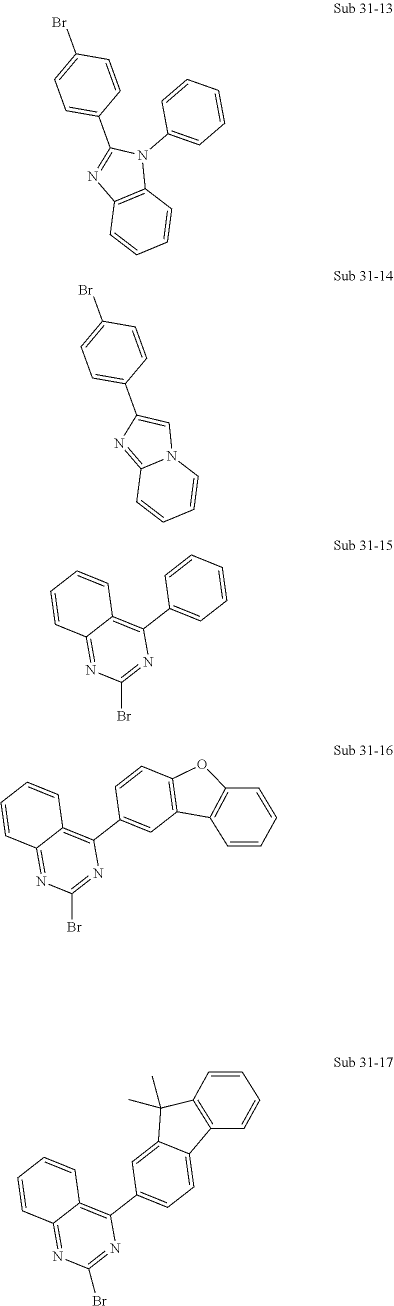

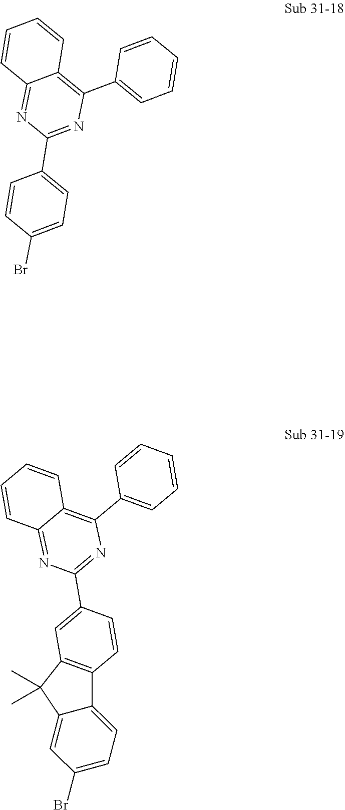 [SMILES: Brc1ccc(-c2nc(-c3ccccc3)c3ccccc3n2)cc1 ; CC1(C)c2cc(Br)ccc2-c2ccc(-c3nc(-c4ccccc4)c4ccccc4n3)cc21]

TABLE 5

| compound | FD-MS |
|---|---|
| Sub 31-1 | m/z = 155.96 (C₆H₅Br = 157.01) |
| Sub 31-2 | m/z = 205.97 (C₁₀H₇Br = 207.07) |
| Sub 31-3 | m/z = 205.97 (C₁₀H₇Br = 207.07) |
| Sub 31-4 | m/z = 231.99 (C₁₂H₉Br = 233.10) |
| Sub 31-5 | m/z = 309.02 (C₁₇H₁₂BrN = 310.19) |
| Sub 31-6 | m/z = 311.01 (C₁₅H₁₀BrN₃ = 312.16) |
| Sub 31-7 | m/z = 310.01 (C₁₆H₁₁BrN₂ = 311.18) |
| Sub 31-8 | m/z = 310.01 (C₁₆H₁₁BrN₂ = 311.18) |
| Sub 31-9 | m/z = 310.01 (C₁₆H₁₁BrN₂ = 311.18) |
| Sub 31-10 | m/z = 387.04 (C₂₁H₁₄BrN₃ = 388.26) |
| Sub 31-11 | m/z = 386.04 (C₂₂H₁₅BrN₂ = 387.27) |
| Sub 31-12 | m/z = 386.04 (C₂₂H₁₅BrN₂ = 387.27) |
| Sub 31-13 | m/z = 348.03 (C₁₉H₁₃BrN₂ = 349.22) |
| Sub 31-14 | m/z = 271.99 (C₁₃H₉BrN₂ = 273.13) |
| Sub 31-15 | m/z = 283.99 (C₁₄H₉BrN₂ = 285.14) |
| Sub 31-16 | m/z = 374.01 (C₂₀H₁₁BrN₂O = 375.22) |
| Sub 31-17 | m/z = 400.06 (C₂₃H₁₇BrN₂ = 401.30) |
| Sub 31-18 | m/z = 360.03 (C₂₀H₁₃BrN₂ = 361.23) |
| Sub 31-19 | m/z = 476.09 (C₂₉H₂₁BrN₂ = 477.39) |

### Synthesis Example of 2-72

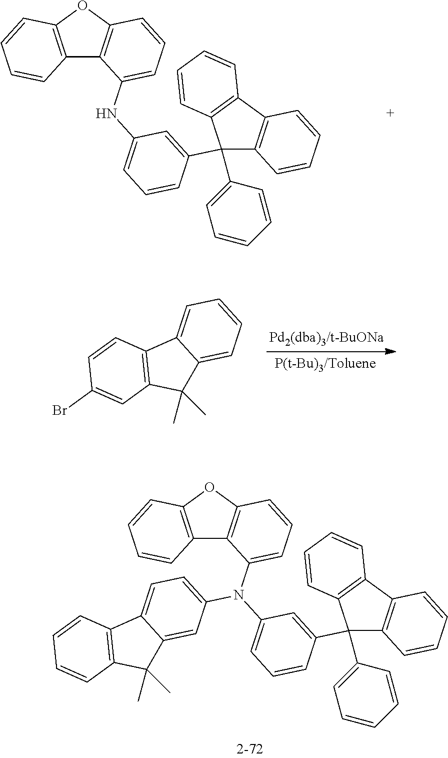 [SMILES: c1ccc(C2(c3cccc(Nc4cccc5oc6ccccc6c45)c3)c3ccccc3-c3ccccc32)cc1 ; CC1(C)c2ccccc2-c2ccc(Br)cc21 ; CC1(C)c2ccccc2-c2ccc(N(c3cccc(C4(c5ccccc5)c5ccccc5-c5ccccc54)c3)c3cccc4oc5ccccc5c34)cc21]

In a round bottom flask, N-(3-(9-phenyl-9H-fluoren-9-yl)phenyl)dibenzo[b,d]furan-1-amine (10.0 g, 20 mmol), 2-bromo-9,9-dimethyl-9H-fluorene (5.5 g, 20 mmol), Pd₂(dba)₃ (0.5 g, 0.6 mmol), P(t-Bu)₃ (0.2 g, 2 mmol), t-BuONa (5.8 g, 60 mmol) and toluene(300 mL) were carried out in the same manner as in 2-34 to give 2-72. (11.1 g, 80%).

### Synthesis Example of 2-81

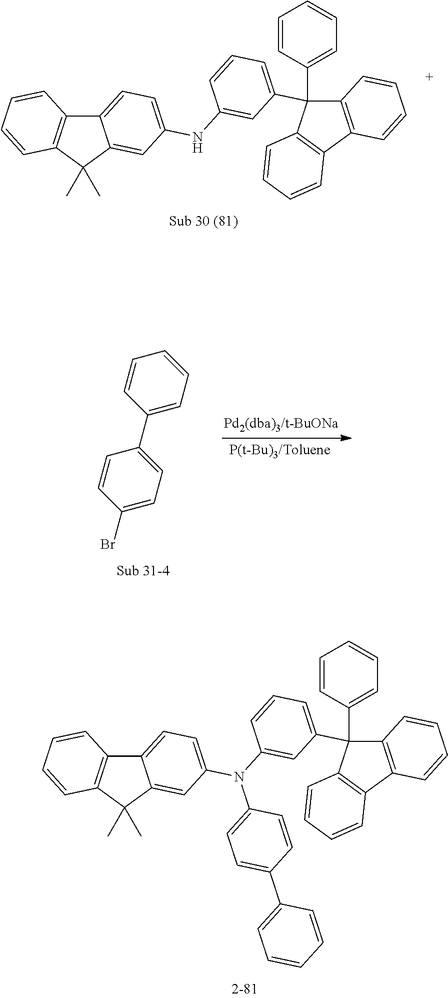 [SMILES: CC1(C)c2ccccc2-c2ccc(Nc3cccc(C4(c5ccccc5)c5ccccc5-c5ccccc54)c3)cc21 ; Brc1ccc(-c2ccccc2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In a round bottom flask, Sub 30(81) (10.3 g, 20 mmol), Sub 31-4 (4.66 g, 20 mmol), Pd₂(dba)₃ (0.5 g, 0.6 mmol), P(t-Bu)₃ (0.2 g, 2 mmol), t-BuONa (5.8 g, 60 mmol) and toluene(300 mL) were carried out in the same manner as in 2-34 to give 2-81. (10.3 g, 76%).

### Synthesis Example of 2-161

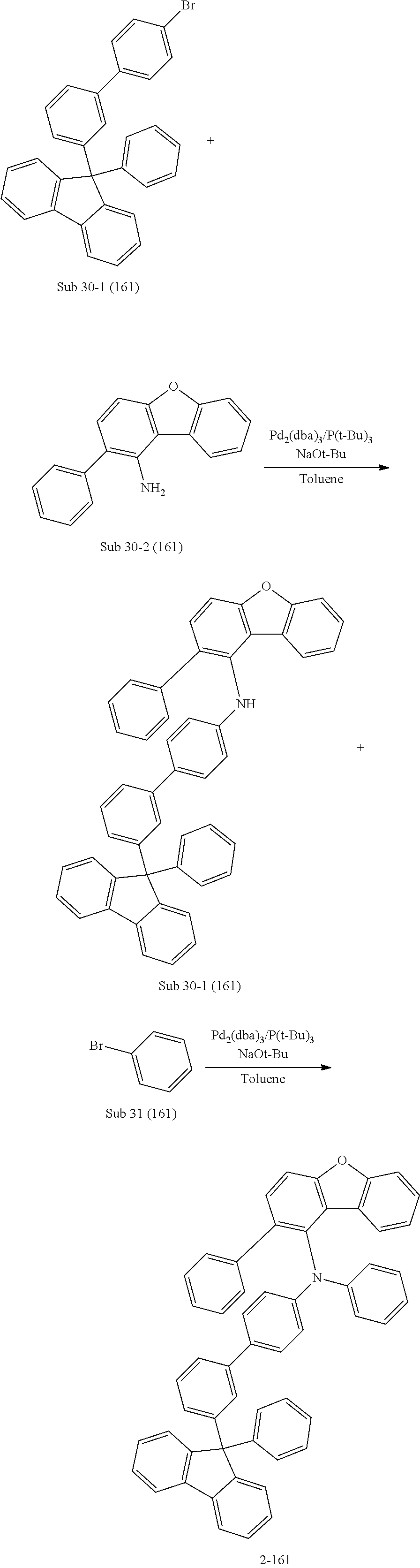 [SMILES: Brc1ccc(-c2cccc(C3(c4ccccc4)c4ccccc4-c4ccccc43)c2)cc1 ; Nc1c(-c2ccccc2)ccc2oc3ccccc3c12 ; c1ccc(-c2ccc3oc4ccccc4c3c2Nc2ccc(-c3cccc(C4(c5ccccc5)c5ccccc5-c5ccccc54)c3)cc2)cc1 ; Brc1ccccc1 ; c1ccc(-c2ccc3oc4ccccc4c3c2N(c2ccccc2)c2ccc(-c3cccc(C4(c5ccccc5)c5ccccc5-c5ccccc54)c3)cc2)cc1]

1) Sub 30(161)

In a round bottom flask, Sub 30-1 (161) (20.0 g, 42.2 mmol) and Sub 30-2 (161) (11.0 g, 42.2 mmol), Pd₂(dba)₃ (1.2 g, 1.3 mmol), P(t-Bu)₃ (0.5 g, 2.5 mmol), NaOt-Bu (8.1 g, 84.5 mmol), toluene (211 mL) were carried out in the same manner as in 2-3 to give Sub 30 (161). (19.7 g, 71.6%).
2) 2-161

In a round bottom flask, Sub 30 (161) (10.0 g, 15.3 mmol) and Sub 31 (161) (2.4 g, 15.3 mmol), Pd₂(dba)₃ (0.4 g, 0.5 mmol), P(t-Bu)₃ (0.2 g, 0.9 mmol), NaOt-Bu (2.9 g, 30.6 mmol), toluene (76 mL) were carried out in the same manner as in 2-3 to give 2-161. (7.9 g, 70.9%).

### Synthesis Example of 2-166

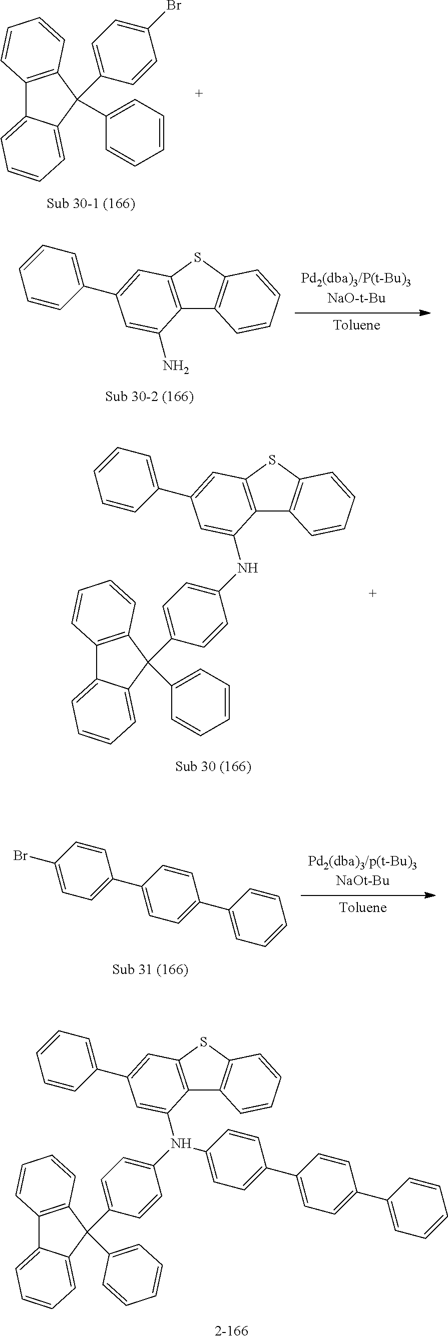 [SMILES: Brc1ccc(C2(c3ccccc3)c3ccccc3-c3ccccc32)cc1 ; Nc1cc(-c2ccccc2)cc2sc3ccccc3c12 ; c1ccc(-c2cc(Nc3ccc(C4(c5ccccc5)c5ccccc5-c5ccccc54)cc3)c3c(c2)sc2ccccc23)cc1 ; Brc1ccc(-c2ccc(-c3ccccc3)cc2)cc1 ; c1ccc(-c2ccc(-c3ccc(N(c4ccc(C5(c6ccccc6)c6ccccc6-c6ccccc65)cc4)c4cc(-c5ccccc5)cc5sc6ccccc6c45)cc3)cc2)cc1]

1) Sub 30 (166)

In a round bottom flask, Sub 30-1 (166) (20.0 g, 50.3 mmol) and Sub 30-2 (166) (13.9 g, 50.3 mmol), Pd₂(dba)₃ (1.4 g, 1.5 mmol), P(t-Bu)₃ (0.6 g, 3.0 mmol), NaOt-Bu (9.7 g, 100.7 mmol), toluene (252 mL) were carried out in the same manner as in 2-3 to give Sub 30 (166). (21.0 g, 70.5%).
2) 2-166

In a round bottom flask, Sub 30 (166) (10.0 g, 16.9 mmol) and Sub 31 (166) (5.2 g, 16.9 mmol), Pd₂(dba)₃ (0.5 g, 0.5 mmol), P(t-Bu)₃ (0.2 g, 1.0 mmol), NaOt-Bu (3.2 g, 33.8 mmol), toluene (84 mL) were carried out in the same manner as in 2-3 to give 2-166. (9.8 g, 71.0%).

### Synthesis Example of 2-167

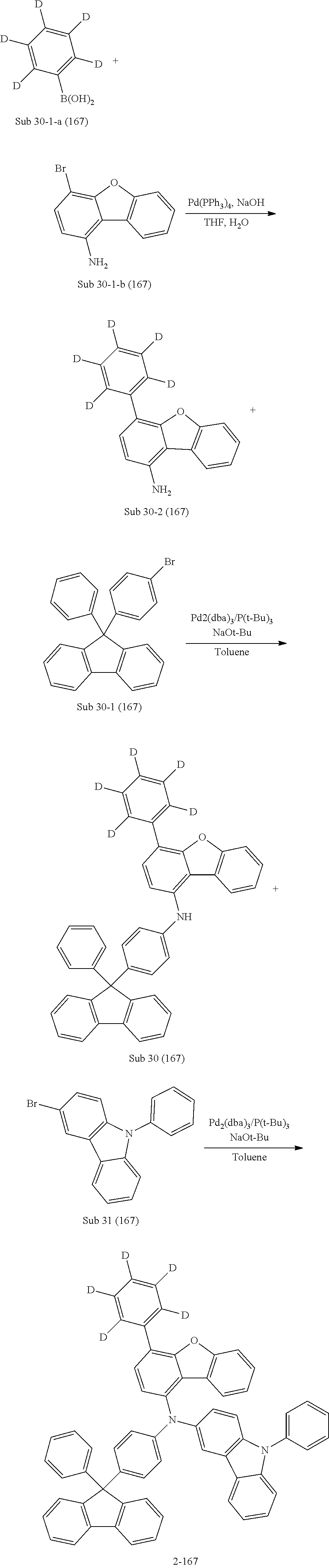

1) Sub 30-2 (167)

In a round bottom flask, Sub 30-1-a (167) (20.0 g, 157.5 mmol) and Sub 30-1-b (167) (41.3 g, 157.5 mmol), Pd(PPh₃)₄ (10.9 g, 9.5 mmol), K₂CO₃ (65.3 g, 472.6 mmol), THF (788 ml), water (394 ml) were carried out in the same manner as in 2-3 to give Sub 30-2 (167). (33.8 g, 81.2%).
2) Sub 30 (167)

In a round bottom flask, Sub 30-1 (167) (20.0 g, 50.3 mmol) and Sub 30-2 (167) (13.3 g, 50.3 mmol), Pd₂(dba)₃ (1.4 g, 1.5 mmol), P(t-Bu)₃ (0.6 g, 3.0 mmol), NaOt-Bu (9.7 g, 100.7 mmol), toluene (252 mL) were carried out in the same manner as in 2-3 to give Sub 30 (167). (20.7 g, 70.7%).
3) 2-167

In a round bottom flask, Sub 30 (167) (10.0 g, 17.2 mmol) [symbol] Sub 31 (167) (5.5 g, 17.2 mmol), Pd₂(dba)₃ (0.5 g, 0.5 mmol), P(t-Bu)₃ (0.2 g, 1.0 mmol), NaOt-Bu (3.3 g, 34.4 mmol), toluene (86 mL) were carried out in the same manner as in 2-3 to give 2-167. (10.1 g, 71.3%).

### Synthesis Example of 2-180

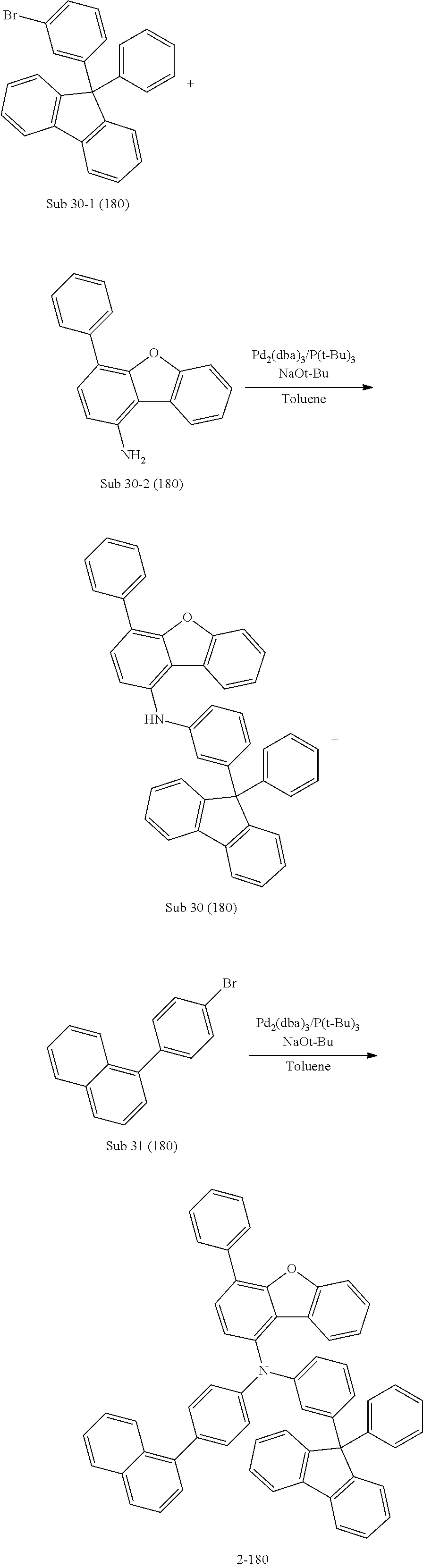 [SMILES: Brc1cccc(C2(c3ccccc3)c3ccccc3-c3ccccc32)c1 ; Nc1ccc(-c2ccccc2)c2oc3ccccc3c12 ; c1ccc(-c2ccc(Nc3cccc(C4(c5ccccc5)c5ccccc5-c5ccccc54)c3)c3c2oc2ccccc23)cc1 ; Brc1ccc(-c2cccc3ccccc23)cc1 ; c1ccc(-c2ccc(N(c3ccc(-c4cccc5ccccc45)cc3)c3cccc(C4(c5ccccc5)c5ccccc5-c5ccccc54)c3)c3c2oc2ccccc23)cc1]

1) Sub 30 (180)

In a round bottom flask, Sub 30-1 (180) (20.0 g, 50.3 mmol) and Sub 30-2 (180) (13.1 g, 50.3 mmol), Pd₂(dba)₃ (1.4 g, 1.5 mmol), P(t-Bu)₃ (0.6 g, 3.0 mmol), NaOt-Bu (9.7 g, 100.7 mmol), toluene (252 mL) were carried out in the same manner as in 2-3 to give Sub 30 (180). (20.7 g, 71.3%).
2) 2-180

In a round bottom flask, Sub 30 (180) (10.0 g, 17.4 mmol) and Sub 31 (180) (4.9 g, 17.4 mmol), Pd₂(dba)₃ (0.5 g, 0.5 mmol), P(t-Bu)₃ (0.2 g, 1.0 mmol), NaOt-Bu (3.3 g, 34.7 mmol), toluene (87 mL) were carried out in the same manner as in 2-3 to give 2-180. (9.8 g, 72.4%).

TABLE 6

| Compound | FD-MS |
|---|---|
| 2-81 | m/z = 677.31 (C₅₂H₃₉N = 677.89) |
| 2-82 | m/z = 651.26 (C₄₉H₃₃NO = 651.81) |
| 2-83 | m/z = 641.22 (C₄₇H₃₁NS = 641.83) |
| 2-84 | m/z = 700.29 (C₅₃H₃₆N₂ = 700.89) |
| 2-85 | m/z = 677.31 (C₅₂H₃₉N = 677.89) |
| 2-86 | m/z = 651.26 (C₄₉H₃₃NO = 651.81) |
| 2-87 | m/z = 843.30 (C₆₃H₄₁NS = 844.09) |
| 2-88 | m/z = 701.28 (C₅₂H₃₅N₃ = 701.87) |
| 2-89 | m/z = 677.31 (C₅₂H₃₉N = 677.89) |
| 2-90 | m/z = 729.28 (C₅₃H₃₅N₃O = 729.88) |
| 2-91 | m/z = 912.29 (C₆₄H₄₀N₄OS = 913.11) |
| 2-92 | m/z = 878.34 (C₆₅H₄₂N₄ = 879.08) |
| 2-93 | m/z = 805.35 (C₆₀H₄₃N₃ = 806.03) |
| 2-94 | m/z = 906.34 (C₆₆H₄₂N₄O = 907.09) |
| 2-95 | m/z = 769.26 (C₅₅H₃₅N₃S = 769.97) |
| 2-96 | m/z = 884.30 (C₆₃H₄₀N₄S = 885.10) |
| 2-97 | m/z = 767.36 (C₅₉H₄₅N = 768.02) |
| 2-98 | m/z = 815.32 (C₆₂H₄₁NO = 816.02) |
| 2-99 | m/z = 829.28 (C₆₂H₃₉NS = 830.06) |
| 2-100 | m/z = 781.35 (C₅₉H₃₅D₅N₂ = 782.01) |
| 2-101 | m/z = 695.30 (C₅₂H₃₈FN = 695.88) |
| 2-102 | m/z = 753.34 (C₅₈H₄₃N = 753.99) |
| 2-103 | m/z = 803.36 (C₆₂H₄₅N = 804.05) |
| 2-104 | m/z = 6829.37 (C₆₄H₄₇N = 830.09) |
| 2-105 | m/z = 918.40 (C₇₀H₅₀N₂ = 919.18) |
| 2-106 | m/z = 601.28 (C₄₆H₃₅N = 601.79) |
| 2-107 | m/z = 677.31 (C₅₂H₃₉N = 677.89) |
| 2-108 | m/z = 753.34 (C₅₈H₄₃N = 753.99) |
| 2-109 | m/z = 701.31 (C₅₄H₃₉N = 701.91) |
| 2-110 | m/z = 677.31 (C₅₂H₃₉N = 677.89) |
| 2-111 | m/z = 677.31 (C₅₂H₃₉N = 677.89) |
| 2-112 | m/z = 682.34 (C₅₂H₃₄D₅N = 682.92) |
| 2-113 | m/z = 701.31 (C₅₄H₃₉N = 701.91) |
| 2-114 | m/z = 619.27 (C₄₆H₃₄FN = 619.78) |
| 2-115 | m/z = 753.34 (C₅₈H₄₃N = 753.99) |
| 2-116 | m/z = 803.36 (C₆₂H₄₅N = 804.05) |
| 2-117 | m/z = 701.31 (C₅₄H₃₉N = 701.91) |
| 2-118 | m/z = 651.29 (C₅₀H₃₇N = 651.85) |
| 2-119 | m/z = 727.32 (C₅₆H₄₁N = 727.95) |
| 2-120 | m/z = 758.37 (C₅₈H₃₈D₅N = 759.02) |
| 2-121 | m/z = 757.37 (C₅₈H₄₇N = 758.02) |
| 2-122 | m/z = 753.34 (C₅₈H₄₃N = 753.99) |
| 2-123 | m/z = 803.36 (C₆₂H₄₅N = 804.05) |
| 2-124 | m/z = 834.40 (C₆₄H₄₂D₅N = 835.12) |
| 2-125 | m/z = 883.33 (C₆₆H₄₅NS = 884.15) |
| 2-126 | m/z = 651.26 (C₄₉H₃₃NO = 651.81) |
| 2-127 | m/z = 641.22 (C₄₇H₃₁NS = 641.83) |
| 2-128 | m/z = 700.29 (C₅₃H₃₆N₂ = 700.89) |
| 2-129 | m/z = 677.31 (C₅₂H₃₉N = 677.89) |
| 2-130 | m/z = 651.26 (C₄₉H₃₃NO = 651.81) |
| 2-131 | m/z = 843.30 (C₆₃H₄₁NS = 844.09) |
| 2-132 | m/z = 701.28 (C₅₂H₃₅N₃ = 701.87) |
| 2-133 | m/z = 677.31 (C₅₂H₃₉N = 677.89) |
| 2-134 | m/z = 729.28 (C₅₃H₃₅N₃O = 729.88) |
| 2-135 | m/z = 912.29 (C₆₄H₄₀N₄OS = 913.11) |
| 2-136 | m/z = 878.34 (C₆₅H₄₂N₄ = 879.08) |
| 2-137 | m/z = 805.35 (C₆₀H₄₃N₃ = 806.03) |
| 2-138 | m/z = 906.34 (C₆₆H₄₂N₄O = 907.09) |
| 2-139 | m/z = 769.26 (C₅₅H₃₅N₃S = 769.97) |
| 2-140 | m/z = 884.30 (C₆₃H₄₀N₄S = 885.10) |
| 2-141 | m/z = 767.36 (C₅₉H₄₅N = 768.02) |
| 2-142 | m/z = 815.32 (C₆₂H₄₁NO = 816.02) |
| 2-143 | m/z = 829.28 (C₆₂H₃₉NS = 830.06) |
| 2-144 | m/z = 781.35 (C₅₉H₃₅D₅N₂ = 782.01) |
| 2-145 | m/z = 695.30 (C₅₂H₃₈FN = 695.88) |
| 2-146 | m/z = 753.34 (C₅₈H₄₃N = 753.99) |
| 2-147 | m/z = 803.36 (C₆₂H₄₅N = 804.05) |
| 2-148 | m/z = 6829.37 (C₆₄H₄₇N = 830.09) |
| 2-149 | m/z = 918.40 (C₇₀H₅₀N₂ = 919.18) |
| 2-150 | m/z = 717.34 (C₅₅H₄₃N = 717.96) |
| 2-151 | m/z = 717.34 (C₅₅H₄₃N = 717.96) |
| 2-152 | m/z = 717.34 (C₅₅H₄₃N = 717.96) |
| 2-153 | m/z = 717.34 (C₅₅H₄₃N = 717.96) |

Although the above has been described with respect to exemplary synthetic examples of the compounds of the present invention, they are all based on the Buchwald-Hartwig cross coupling reaction, Miyaura boration reaction, Suzuki cross-coupling reaction, Intramolecular acid-induced cyclization reaction (J. mater. Chem. 1999, 9, 2095), Pd(II)-catalyzed oxidative cyclization reaction (Org. Lett. 2011, 13, 5504) and the PPh₃-mediated reductive cyclization reaction (J. Org. Chem. 2005, 70, 5014), and it will be easily understood by those skilled in the art that the above reaction proceeds even if a substituent other than the substituent specified in the specific synthetic examples is combined.

### Evaluation of Manufacture of Organic Electronic Element

### Example 5) Manufacture and Evaluation of Blue Organic Light Emitting Diode

First, on an ITO layer(anode) formed on a glass substrate, N¹-(naphthalen-2-yl)-N⁴, N⁴-bis(4-(naphthalen-2-yl(phenyl)amino)phenyl)-N¹-phenylbenzene-1,4-diamine (hereinafter will be abbreviated as 2-TNATA) was vacuum-deposited to form a hole injection layer with a thickness of 60 nm. Subsequently, and on the layer, 4,4-bis[N-(1-naphthyl)-N-phenylamino]biphenyl(hereinafter will be abbreviated as NPD) was vacuum-deposited to a thickness of 60 nm to form a hole transport layer. Subsequently, the inventive compound was vacuum-deposited as an emitting auxiliary layer material to a thickness of 20 nm to form an emitting auxiliary layer. After forming the emitting auxiliary layer, on the emitting auxiliary layer, 9,10-di(naphthalen-2-yl)anthracene is used as a host, and BD-052X(Idemitsu kosan) is used as dopant in a ratio of 96:4, therefore an emitting layer with a thickness of 30 nm was deposited on the emitting auxiliary layer. (1,1′-bisphenyl)-4-olato)bis(2-methyl-8-quinolinolato)aluminum (hereinafter abbreviated as BAlq) was vacuum deposited as a hole blocking layer to a thickness of 10 nm, and Tris(8-quinolinol) aluminum (hereinafter abbreviated as Alq3) was deposited to a thickness of 40 nm as an electron transport layer. After that, an alkali metal halide, LiF was vacuum deposited as an electron injection layer to a thickness of 0.2 nm, and Al was deposited to a thickness of 150 nm to form a cathode to manufacture an OLED.

To the OLEDs which were manufactured by examples and comparative examples, a forward bias direct current voltage was applied, and electroluminescent (EL) properties were measured using PR-650 of Photoresearch Co., and T95 life was measured using a life measuring apparatus manufactured by McScience Inc. with a reference luminance of 5000 cd/m². In the following table, the manufacture of a device and the results of evaluation are shown.

To the OLEDs which were manufactured by examples and comparative examples, a forward bias direct current voltage was applied, and electroluminescent (EL) properties were measured using PR-650 of Photoresearch Co., and T95 life was measured using a life measuring apparatus manufactured by McScience Inc. with a reference luminance of 5000 cd/m². In the following table, the manufacture of a device and the results of evaluation are shown.

### Comparative Examples 35˜37

An OLED was prepared in the same manner as in Example 5 except that the emitting auxiliary layer was not used and Comparative Compound F, Comparative Compound G and Invention Compound 2-81 were used as the hole transport layer material.

### Comparative Example 38

An OLED was prepared in the same manner as in Example 5 except that the emitting auxiliary layer was not used.

### Comparative Examples 39˜40

An OLED was prepared in the same manner as in Example 5 except that the Comparative Compound F or Comparative Compound G were used as the emitting auxiliary layer material.

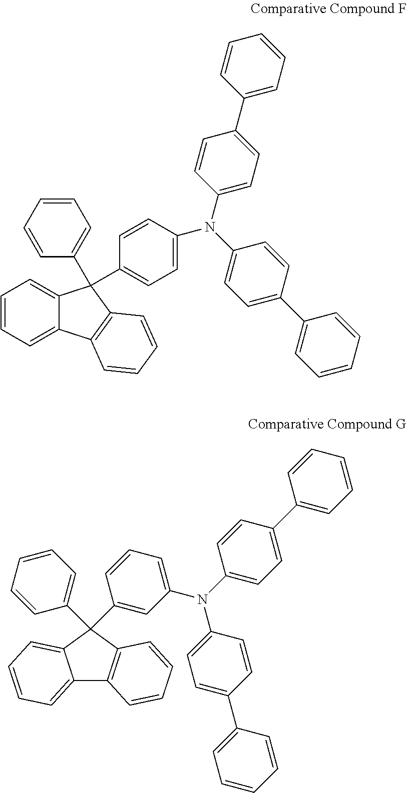 [SMILES: c1ccc(-c2ccc(N(c3ccc(-c4ccccc4)cc3)c3ccc(C4(c5ccccc5)c5ccccc5-c5ccccc54)cc3)cc2)cc1 ; c1ccc(-c2ccc(N(c3ccc(-c4ccccc4)cc3)c3cccc(C4(c5ccccc5)c5ccccc5-c5ccccc54)c3)cc2)cc1]

TABLE 11

| | | Emitting | | | | | |
|---|---|---|---|---|---|---|---|
| | Hole | auxiliary | | Current | bright- | | |
| | transport | layer | volt- | density | ness | Efficiency | |
| | compound | compound | age | (mA/cm²) | (cd/m²) | (cd/A) | T(95) |
| comparative | comparative | — | 6.8 | 14.3 | 500.0 | 3.5 | 70.4 |
| example (35) | compound F | | | | | | |
| comparative | comparative | | 7.0 | 15.2 | 500.0 | 3.3 | 69.6 |
| example (36) | compound G | | | | | | |
| comparative | 2-81 | | 6.9 | 13.5 | 500.0 | 3.7 | 71.2 |
| example (37) | | | | | | | |
| comparative | NPB | — | 7.3 | 17.9 | 500.0 | 2.8 | 65.3 |
| example (38) | | | | | | | |
| comparative | | comparative | 6.8 | 11.1 | 500.0 | 4.5 | 80.4 |
| example (39) | | compound F | | | | | |
| comparative | | comparative | 6.5 | 10.2 | 500.0 | 4.9 | 81.7 |
| example (40) | | compound G | | | | | |
| example(57) | | 2-81 | 6.3 | 9.1 | 500.0 | 5.5 | 94.9 |
| example(58) | | 2-83 | 6.2 | 8.6 | 500.0 | 5.8 | 97.0 |
| example(59) | | 2-85 | 6.1 | 9.2 | 500.0 | 5.4 | 93.2 |
| example(60) | | 2-86 | 6.2 | 8.9 | 500.0 | 5.6 | 96.9 |
| example(61) | | 2-89 | 6.1 | 9.3 | 500.0 | 5.4 | 94.1 |
| example(62) | | 2-107 | 6.1 | 9.3 | 500.0 | 5.4 | 94.8 |
| example(63) | | 2-122 | 6.0 | 9.1 | 500.0 | 5.5 | 94.7 |
| example(64) | | 2-127 | 6.2 | 8.8 | 500.0 | 5.7 | 96.0 |
| example(65) | | 2-128 | 6.1 | 8.7 | 500.0 | 5.7 | 95.6 |
| example(66) | | 2-133 | 6.1 | 9.4 | 500.0 | 5.3 | 94.3 |
| example(67) | | 2-150 | 6.2 | 9.2 | 500.0 | 5.4 | 93.6 |
| example(68) | | 2-151 | 6.0 | 9.4 | 500.0 | 5.3 | 94.1 |
| example(69) | | 2-152 | 6.0 | 9.1 | 500.0 | 5.5 | 93.7 |
| example(70) | | 2-153 | 6.1 | 9.4 | 500.0 | 5.3 | 93.2 |

### Example 6) Manufacture and Evaluation of Green Organic Light Emitting Diode

First, on an ITO layer (anode) formed on a glass substrate, N¹-(naphthalen-2-yl)-N⁴,N⁴-bis(4-(naphthalen-2-yl(phenyl)amino)phenyl)-N¹-phenylbenzene-1,4-diamine (hereinafter will be abbreviated as 2-TNATA) was vacuum-deposited to form a hole injection layer with a thickness of 60 nm. Subsequently, and on the layer, 4,4-bis[N-(1-naphthyl)-N-phenylamino]biphenyl(hereinafter will be abbreviated as NPD) was vacuum-deposited as a hole transport compound to a thickness of 60 nm to form a hole transport layer. Subsequently, the inventive compound represented by Formula (30) was vacuum-deposited as an emitting auxiliary layer material to a thickness of 20 nm to form an emitting auxiliary layer. After forming the emitting auxiliary layer, on the emitting auxiliary layer, 4,4′-di(9H-carbazol-9-yl)-1,1′-biphenyl is used as a host, and Ir(ppy)3[tris(2-phenylpyridine)-iridium] is used as dopant in a ratio of 95:5, and an emitting layer with a thickness of 30 nm was deposited. (1,1′-bisphenyl)-4-olato)bis(2-methyl-8-quinolinolato)aluminum(hereinafter abbreviated as BAlq) was vacuum deposited as a hole blocking layer to a thickness of 10 nm, and Tris (8-quinolinol) aluminum (hereinafter abbreviated as Alq3) was deposited to a thickness of 40 nm as an electron transport layer. After that, an alkali metal halide, LiF was vacuum deposited as an electron injection layer to a thickness of 0.2 nm, and Al was deposited to a thickness of 150 nm to form a cathode to manufacture an OLED.

To the OLEDs which were manufactured by examples and comparative examples, a forward bias direct current voltage was applied, and electroluminescent (EL) properties were measured using PR-650 of Photoresearch Co., and T95 life was measured using a life measuring apparatus manufactured by McScience Inc. with a reference luminance of 5000 cd/m². In the following table, the manufacture of a device and the results of evaluation are shown.

### Comparative Examples 41˜42

An OLED was prepared in the same manner as in Example 6 except that Comparative Compound F or Comparative Compound G were used as the emitting auxiliary layer material.

TABLE 12

| | | | Current | bright- | | | | |
|---|---|---|---|---|---|---|---|---|
| | | | density | ness | Efficiency | | CIE | |
| | compound | voltage | (mA/cm²) | (cd/m²) | (cd/A) | T(95) | x | y |
| comparative | comparative | 7.3 | 17.4 | 5000.0 | 28.7 | 80.4 | 0.30 | 0.61 |
| example(41) | compound F | | | | | | | |
| comparative | comparative | 7.4 | 19.9 | 5000.0 | 25.1 | 80.9 | 0.33 | 0.64 |
| example(42) | compound G | | | | | | | |
| example(71) | 2-81 | 5.6 | 11.0 | 5000.0 | 45.4 | 120.3 | 0.31 | 0.64 |
| example(72) | 2-83 | 6.3 | 14.2 | 5000.0 | 35.1 | 104.3 | 0.31 | 0.64 |
| example(73) | 2-85 | 5.8 | 13.8 | 5000.0 | 36.2 | 110.1 | 0.34 | 0.61 |
| example(74) | 2-86 | 6.4 | 14.3 | 5000.0 | 35.0 | 103.6 | 0.33 | 0.63 |
| example(75) | 2-89 | 6.0 | 14.1 | 5000.0 | 35.5 | 113.0 | 0.33 | 0.65 |
| example(76) | 2-107 | 5.8 | 11.3 | 5000.0 | 44.3 | 112.6 | 0.32 | 0.65 |
| example(77) | 2-122 | 5.9 | 11.4 | 5000.0 | 43.8 | 112.3 | 0.34 | 0.63 |
| example(78) | 2-127 | 6.4 | 12.6 | 5000.0 | 39.8 | 104.2 | 0.33 | 0.65 |
| example(79) | 2-128 | 6.3 | 12.8 | 5000.0 | 39.2 | 102.5 | 0.32 | 0.65 |
| example(80) | 2-133 | 5.9 | 12.5 | 5000.0 | 39.9 | 111.2 | 0.32 | 0.65 |
| example(81) | 2-150 | 5.4 | 11.4 | 5000.0 | 43.8 | 114.8 | 0.31 | 0.64 |
| example(82) | 2-151 | 5.2 | 11.2 | 5000.0 | 44.8 | 111.5 | 0.30 | 0.63 |
| example(83) | 2-152 | 5.2 | 11.6 | 5000.0 | 43.1 | 112.1 | 0.31 | 0.63 |
| example(84) | 2-153 | 5.3 | 11.2 | 5000.0 | 44.8 | 114.4 | 0.30 | 0.62 |

As can be seen from the results of Table 11 to 12, when OLED was manufactured by using the material for an organic electroluminescence device of the present invention as an emitting auxiliary layer material, the driving voltage of the organic electroluminescent device can be lowered and the luminous efficiency and lifetime can be remarkably improved as compared with the comparative example not using the material for the emitting auxiliary layer or using the comparative compound F or the comparative compound G.

Table 11 shows the results of the production of a blue organic light emitting device. It can be confirmed that excellent results are obtained when the compound of the present invention is used as an emitting auxiliary layer. The results of Comparative Example 39 or Comparative Example 40 and Examples 57 to 70 show that compounds of the present invention substituted with specific substituents such as DBT, DBF, Cz, and Fluorene are remarkably superior to the comparative compounds substituted with the general aryl group even though the mother compound is similar. That is, when specific substituents such as DBT, DBF, Cz, and Fluorene are introduced, the refractive index, the Tg, and the energy level of the compound (HOMO, LUMO, T1, etc.) become significantly different, and this difference in physical properties is a major factor in improving the device performance during device deposition (for example, such as an energy balance), such that different device results can be derived.

Table 12 shows the results of the production of a green organic light emitting device. When the compound of the present invention was used as an emitting auxiliary layer, the results were significantly superior to the comparative compounds. This is also the effect of certain substituents such as DBT, DBF, Cz, and Fluorene, and a specific feature is that the superiority of the green auxiliary layer is significantly improved than the blue auxiliary layer. Further, in the case of the blue auxiliary layer, DBT and DBF substituted compounds showed the most excellent properties, but the results of the green auxiliary layer showed the best results with the Fluorene substituted compounds. This suggests that even if the emitting auxiliary layer compound is the same, the properties required depending on the color of the emitting layer are different, so that a result which can not be deduced by those skilled in the art can be obtained.

### Evaluation of Manufacture of Organic Electronic Element

### Experimental Example 7) Manufacture and Evaluation of Red Organic Light Emitting Diode

### [Example 85] Red Organic Electroluminescent Device (Emitting Auxiliary Layer)

Compound A and Compound B were used on the ITO layer (anode) formed on a glass substrate, and Compound B was doped at a weight ratio of 98:2 to form a hole injection layer with a thickness of 10 nm. Then, Compound A was vacuum-deposited on the hole injection layer with a thickness of 110 nm to form a hole transport layer.

Next, compound 2-72 of the present invention was vacuum-deposited to a thickness of 10 nm on the hole transport layer to form an emitting auxiliary layer. Afterwards, compound D-R was used as the host material of the emitting layer, and bis-(1-phenylisoquinolyl)iridium(III)acetylacetonate (hereinafter ‘(piq)₂Ir (acac)’) was used as the dopant material, and the dopant was doped so that the weight ratio of the host and dopant was 95:5 to form an emitting layer with a thickness of 30 nm.

Next, compound E was vacuum-deposited on the emitting layer to form a hole blocking layer with a thickness of 10 nm, and a mixture of compound F and compound G at a weight ratio of 5:5 was used on the hole blocking layer to form an electron transport layer with a thickness of 30 nm. Afterwards, compound G was deposited on the electron transport layer to form an electron injection layer with a thickness of 0.2 nm, and then Al was deposited to form a cathode with a thickness of 150 nm.
compound A: N-([1,1′-biphenyl]-4-yl)-9,9-dimethyl-N-(4-(9-phenyl-9H-carbazol-3-yl)phenyl)-9H-fluoren-2-amine
compound B: 4,4′,4″-((1E,1′E,1″E)-cyclopropane-1,2,3-triylidenetris(cyanomethaneylylidene))tris(2,3,5,6-tetrafluorobenzonitrile) compound D-R: 14-(4-phenylquinazolin-2-yl)-14H-benzo[c]benzo[4,5]thieno[2,3-a]carbazole
compound E: 2-(4′-(9,9-dimethyl-9H-fluoren-2-yl)-[1,1′-biphenyl]-3-yl)-4,6-diphenyl-1,3,5-triazine
compound F: 2,7-bis(4-(4,6-diphenyl-1,3,5-triazin-2-yl)phenyl)naphthalene
compound G: (8-quinolinolato)lithium

[Example 86] to [Example 100] An organic electroluminescent device was manufactured in the same manner as Example 85, except that the compound of the present invention described in Table 13 was used instead of the compound 2-86 of the present invention as an emitting auxiliary layer material.

### Comparative Example 43 and Comparative Example 44

An organic electroluminescent device was manufactured in the same manner as Example 85, except that Comparative Compound H or Comparative Compound I was used instead of Compound 2-86 of the present invention as an emitting auxiliary layer material.

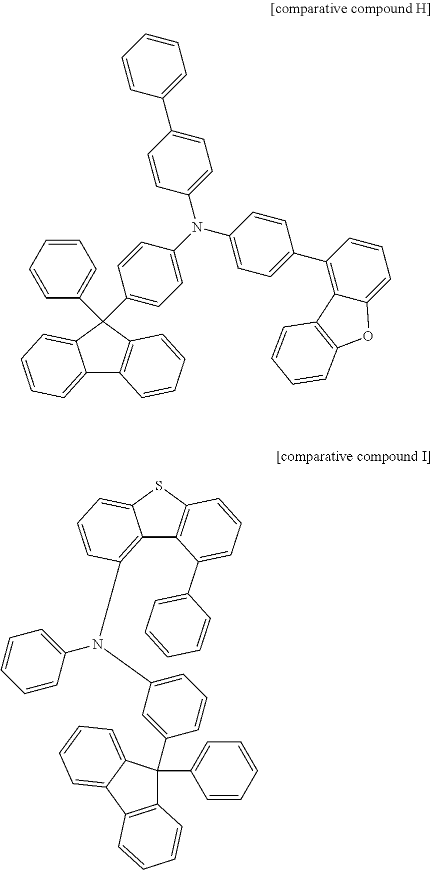 [SMILES: c1ccc(-c2ccc(N(c3ccc(-c4cccc5oc6ccccc6c45)cc3)c3ccc(C4(c5ccccc5)c5ccccc5-c5ccccc54)cc3)cc2)cc1 ; c1ccc(-c2cccc3sc4cccc(N(c5ccccc5)c5cccc(C6(c7ccccc7)c7ccccc7-c7ccccc76)c5)c4c23)cc1]

Electroluminescence (EL) characteristics were measured using PR-650 from Photoresearch by applying a forward bias DC voltage to the organic electroluminescence devices manufactured by Examples 85 to 100 of the present invention, Comparative Examples 43 and 44. As a result of the measurement, the T95 lifespan was measured using a lifespan measuring apparatus manufactured by McScience Inc. with a reference luminance of 2,500 cd/m². Table 13 shows the results of the device fabrication and evaluation.

The measuring apparatus allows the performance of new materials to be evaluated against reference compounds under identical conditions, without being affected by possible daily variations in deposition rate, vacuum quality or other parameters.

Since, during the evaluation, one batch contains 4 identically prepared OLEDs including a comparative compound, and the performance of a total of 12 OLEDs is evaluated in 3 batches, the values of the experimental results obtained in this way exhibit statistical significance.

TABLE 13

| | | | Current | | | |
|---|---|---|---|---|---|---|
| | | | density | brightness | Efficiency | |
| | compound | voltage | (mA/cm²) | (cd/m²) | (cd/A) | T(95) |
| comparative | comparative | 5.3 | 14.4 | 2500.0 | 17.4 | 93.5 |
| example 43 | compound | | | | | |
| | H | | | | | |
| comparative | comparative | 5.2 | 14.8 | 2500.0 | 16.9 | 89.7 |
| example 44 | compound I | | | | | |
| example 85 | 2-72 | 4.5 | 8.2 | 2500.0 | 30.4 | 110.6 |
| example 86 | 2-154 | 4.4 | 6.9 | 2500.0 | 36.3 | 117.7 |
| example 87 | 2-156 | 4.3 | 6.9 | 2500.0 | 36.0 | 124.6 |
| example 88 | 2-158 | 4.3 | 7.0 | 2500.0 | 35.7 | 121.7 |
| example 89 | 2-163 | 4.1 | 6.3 | 2500.0 | 39.9 | 129.0 |
| example 90 | 2-164 | 4.2 | 6.5 | 2500.0 | 38.3 | 128.4 |
| example 91 | 2-167 | 4.3 | 6.6 | 2500.0 | 37.6 | 127.5 |
| example 92 | 2-170 | 4.3 | 6.7 | 2500.0 | 37.4 | 127.2 |
| example 93 | 2-171 | 4.2 | 6.5 | 2500.0 | 38.7 | 128.1 |
| example 94 | 2-173 | 4.3 | 7.4 | 2500.0 | 34.0 | 121.1 |
| example 95 | 2-178 | 4.3 | 7.2 | 2500.0 | 34.8 | 124.8 |
| example 96 | 2-180 | 4.5 | 8.0 | 2500.0 | 31.2 | 111.3 |
| example 97 | 2-181 | 4.5 | 8.2 | 2500.0 | 30.6 | 111.4 |
| example 98 | 2-183 | 4.3 | 7.3 | 2500.0 | 34.4 | 115.9 |
| example 99 | 2-186 | 4.1 | 6.4 | 2500.0 | 39.3 | 129.4 |
| example | 2-187 | 4.3 | 7.5 | 2500.0 | 33.5 | 114.6 |
| 100 | | | | | | |

As can be seen from the results in Table 13, when a red organic electroluminescent device was manufactured using the material for an organic electroluminescent device of the present invention as an emitting auxiliary layer material, the driving voltage, luminescence efficiency, and lifespan of the organic electroluminescence device can be improved compared to the comparative example using Comparative compound H or Comparative compound I having a similar basic skeleton to the compound of the present invention.

Comparative compound H or comparative compound I is similar to the compound of the present invention in that the phenyl moiety of 9,9-diphenylfluorene is substituted with an amine group, and one of the substituents of the amine group comprises 1-dibenzofuran or 1-dibenzothiophene, but Comparative compound H differs from the compound of the present invention in that it necessarily includes a linking group between the amine group and 1-dibenzofuran, and Comparative compound I differs from the compound of the present invention in that a substituent other than hydrogen or deuterium is further substituted at position 9 of dibenzothiophene.

To explain in more detail, the data measured using the DFT method (B3LYP/6-31g (D)) of the Gaussian program to confirm the energy level of the compound due to the structural difference for the compound 2-186 of the present invention, which is structurally similar to Comparative compound H, are as shown in Table 14.

TABLE 14

| | Comparative | |
|---|---|---|
| compound | compound H | 2-186 |
| HOMO(eV) | −4.92 | −4.96 |

As can be seen from the results in Table 14, it can be confirmed that the HOMO value of compound 2-186 of the present invention is deeper than the HOMO Energy Level (hereinafter, HOMO) value of Comparative compound H. As a result, when the compound of the present invention is applied to a device, hole transfer from the emitting auxiliary layer to the emitting layer is facilitated compared to the comparative compound H, so that the hole accumulation in the emitting auxiliary layer is reduced, and as the emitting layer becomes rich in holes, exciton formation becomes easier, so that the charge balance inside the device increases, and the efficiency and lifespan of the device are thought to be significantly improved.

Next, in order to confirm the energy level for compound 2-187 of the present invention, which is structurally similar to Comparative compound I, the data measured using the DFT method (B3LYP/6-31g(D)) of the Gaussian program are as shown in Table 15.

TABLE 15

| | Comparative | |
|---|---|---|
| compound | compound I | 2-187 |
| LUMO(eV) | −1.26 | −1.09 |
| T1(eV) | 2.69 | 2.94 |

As can be seen from the results in Table 15, it can be confirmed that the LUMO value and T1 value of compound 2-187 of the present invention are shallower than the LUMO Energy Level (hereinafter, LUMO) value of Comparative compound I. This shows that even if a compound has a similar structure, the energy level of the compound can significantly change depending on the position where the substituent is substituted. Accordingly, when the compound of the present invention is applied to a device, the blocking effect on electrons passing from the emitting layer is maximized compared to the Comparative compound I, and therefore, the lifespan of the device is maximized.

In addition, among Formula 37 structures of the present invention, when a substituent is substituted at the R24 position, it can be confirmed that the performance of the device is improved compared to the compound without the substituent. It is judged that the device performance is also improved as the stability of the structure is improved by increasing the BDE of the structure itself by substituting another additional substituent on the ring where the amine group is substituted.

That is, as can be seen from the results in Tables 13 to 15, it can be confirmed that the compound satisfying all the structural features and configurations disclosed in the present invention exhibits a remarkable effect in an organic electric device, compared to the comparative compound H and comparative compound I which have a structurally similar configuration to the compound of the present invention. This means that the compound of the present invention satisfying all the specific configurations exhibits a remarkable effect, compared to other comparative compounds not described in this specification.

These results suggest that even for compounds with similar molecular components, depending on the type and position of the substituted substituent, the properties of the compound, such as hole characteristics, light efficiency characteristics, energy level, hole injection and mobility characteristics, charge balance of holes and electrons, volume density, and intermolecular distance, can change significantly to a degree that is difficult to predict, and it also suggests that the performance of a device may vary due to complex factors, rather than a single compound composition influencing the outcome of the entire device.

In the case of an emitting auxiliary layer, the relationship between the hole transport layer and the emitting layer (host) must be understood. Therefore, even if a similar core is used, it would be very difficult for even a person skilled in the art to infer the characteristics exhibited in the emitting auxiliary layer in which the compound of the present invention is used.

Also, the evaluation results of the above-described device fabrication explained the device characteristics in which the compound of the present invention was applied only to the emitting auxiliary layer, but the compound of the present invention can be used by applying it to the hole transport layer or by applying it to both the hole transport layer and the emitting auxiliary layer.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

What is claimed is:

1. A compound represented by Formula (37): 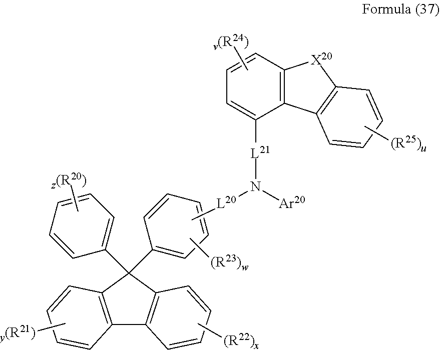
in Formula (37),
1) R²⁰, R²¹, R²², R²³ and R²⁴ are each independently selected from the group consisting of hydrogen; deuterium; halogen; a C₆-C₃₀ aryl group; a fluorenyl group; a C₂-C₃₀ heterocyclic group including at least one heteroatom of O, N, S, Si or P; a fused ring group of a C₃-C₆₀ aliphatic ring and a C₆-C₆₀ aromatic ring; a C₁-C₂₀ alkyl group; a C₂-C₂₀ alkenyl group; a C₂-C₂₀ alkynyl group; a C₁-C₅₀ alkoxyl group; or plurality of R²⁰, plurality of R²¹, plurality of R²², plurality of R²³, and plurality of R²⁴ may be bonded to each other to form an aromatic ring or heteroaromatic ring,
2) R²⁵ is hydrogen; or deuterium; a plurality of R²⁵ may be bonded to each other to form an aromatic ring,
3) v is an integer of 0 to 3, u, w, x and y are each independently an integer of 0 to 4, z is an integer of 0 to 5,
4) L²⁰ is a single bond; a C₆-C₅₀ arylene group; or a C₃-C₃₀ heteroarylene group; L²¹ is a single bond;
5) Ar²⁰ is a C₆-C₅₀ aryl group; or a C₃-C₃₀ heteroaryl group;
6) X²⁰ is O or S,
wherein the aryl group, arylene group, heterocyclic group, fluorenyl group, heteroaryl group, heteroarylene group, fused ring group, alkyl group, alkenyl group, alkynyl group, and alkoxy group may be substituted with one or more substituents selected from the group consisting of deuterium; halogen; a silane group; siloxane group; boron group; germanium group; cyano group; nitro group; a C₁-C₂₀ alkylthio group; C₁-C₂₀ alkoxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group and C₈-C₂₀ arylalkenyl group, wherein the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

2. The compound of claim 1, wherein R²⁴ is a substituent other than H or deuterium.

3. The compound of claim 1, wherein Formula 37 is represented by any one of Formulas 37-1 to 37-3: 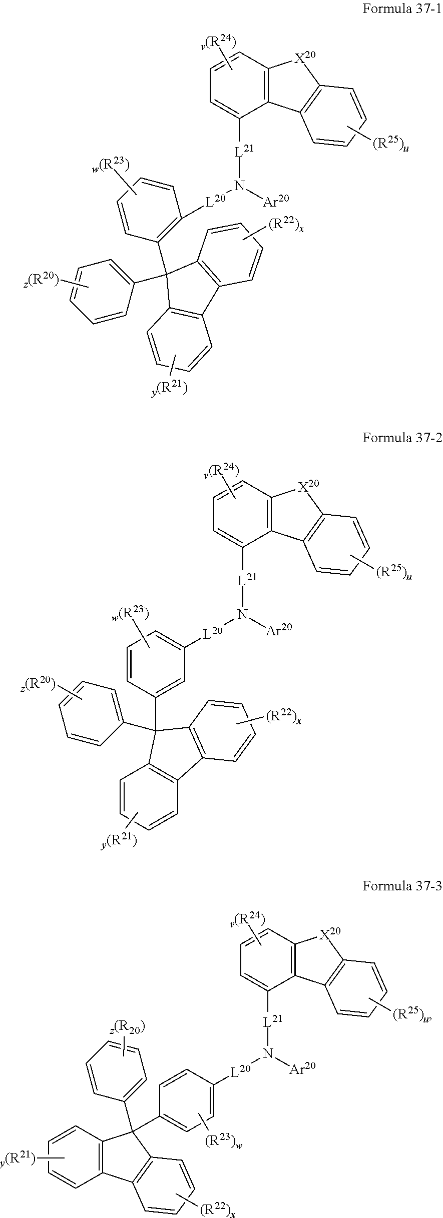
wherein R²⁰, R²¹, R²², R²³, R²⁴, R²⁵, L²⁰, L²¹, Ar²⁰ and X²⁰, u, v, w, x, y and z are the same as defined in Formula 37.

4. The compound of claim 1, wherein Formula 37 is represented by Formulas 37-4 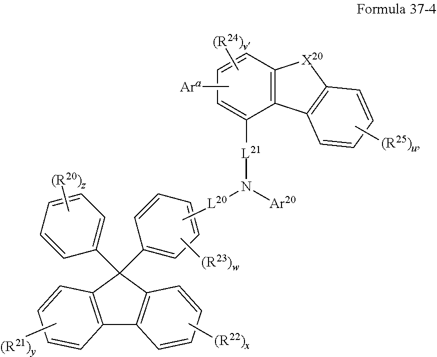
wherein R²⁰, R²¹, R²², R²³, R²⁴, R²⁵, L²⁰, L²¹, Ar²⁰ and X²⁰, u, w, x, y and z are the same as defined in Formula 37,
v′ is an integer of 0 to 2, and
Ar^{a} is a C₆-C₅₀ aryl group; a C₃-C₅₀ heteroaryl group; or a C₁-C₅₀ alkyl group.

5. The compound of claim 1, wherein Ar²⁰ is represented by any one of Formulas Ar-1 to Ar-9: 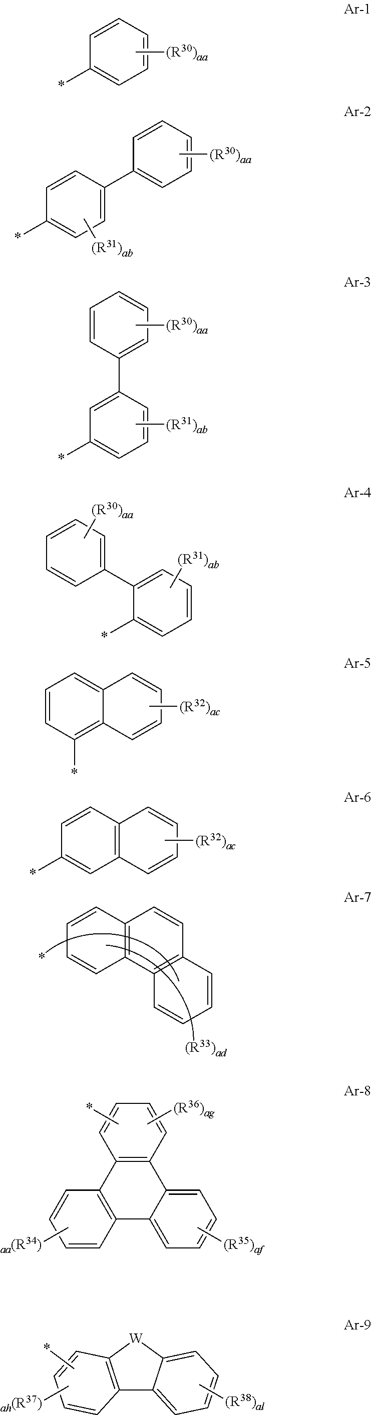
in Formula Ar-1 to Ar-9,
1) R³⁰, R³¹, R³², R³³, R³⁴, R³⁵, R³⁶, R³⁷ and R³⁸ are each independently selected from the group consisting of hydrogen; deuterium; C₁-C₃₀ alkyl group; C₂-C₂₀ alkenyl group; C₂-C₂₀ alkynyl group; a C₁-C₂₀ alkoxy group; a C₆-C₂₀ aryl group; a fluorenyl group; a C₂-C₂₀ heterocyclic group including at least one hetero atom of O, N, S, Si, P; a fused ring group of a C₃-C₆₀ aliphatic ring and a C₆-C₂₀ aromatic ring; and each as plurality are the same as or different from each other, and a plurality of R³⁰ or a plurality of R³¹ or a plurality of R³² or a plurality of R³³ or a plurality of R³⁴ or a plurality of R³⁵ or a plurality of R³⁶ or a plurality of R³⁷ or a plurality of R³⁸ may be bonded to each other to form an aromatic or a heteroaromatic ring,
2) W is O, S, CR⁴¹R⁴² or NR⁴³, wherein R⁴¹, R⁴² and R⁴³ are each independently selected from the group consisting of hydrogen; deuterium; C₁-C₃₀ alkyl group; C₂-C₂₀ alkenyl group; C₂-C₂₀ alkynyl group; a C₁-C₂₀ alkoxy group; a C₆-C₂₀ aryl group; a fluorenyl group; a C₂-C₂₀ heterocyclic group including at least one hetero atom of O, N, S, Si, P; and R⁴¹ and R⁴² may be bonded to each other to form a spiro ring, and
3) aa is an integer of 0 to 5; ab is an integer of 0 to 4; ac is an integer of 0 to 7; ad is an integer of 0 to 9, ae, af and ai are each independently an integer of 0 to 4; ag and ah are each independently an integer of 0 to 3.

6. The compound of claim 1 selected from the group consisting of the following compounds:

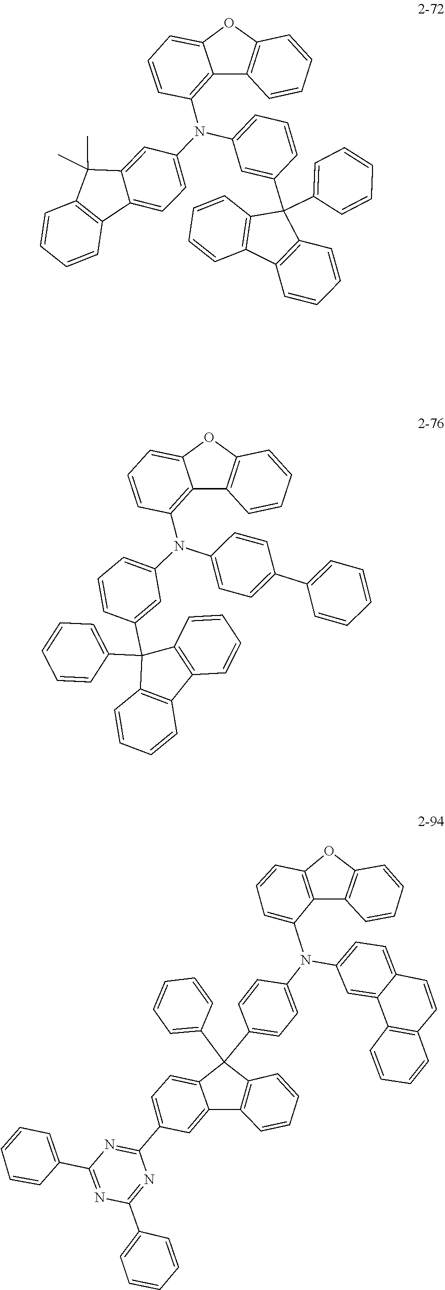 [SMILES: CC1(C)c2ccccc2-c2ccc(N(c3cccc(C4(c5ccccc5)c5ccccc5-c5ccccc54)c3)c3cccc4oc5ccccc5c34)cc21 ; c1ccc(-c2ccc(N(c3cccc(C4(c5ccccc5)c5ccccc5-c5ccccc54)c3)c3cccc4oc5ccccc5c34)cc2)cc1 ; c1ccc(-c2nc(-c3ccccc3)nc(-c3ccc4c(c3)-c3ccccc3C4(c3ccccc3)c3ccc(N(c4ccc5ccc6ccccc6c5c4)c4cccc5oc6ccccc6c45)cc3)n2)cc1]

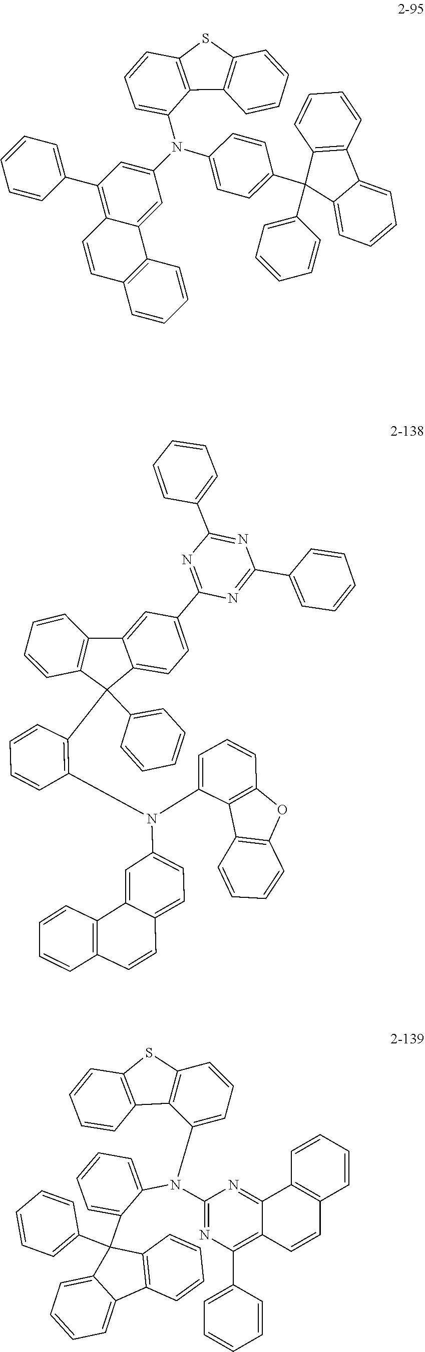 [SMILES: c1ccc(-c2cc(N(c3ccc(C4(c5ccccc5)c5ccccc5-c5ccccc54)cc3)c3cccc4sc5ccccc5c34)cc3c2ccc2ccccc23)cc1 ; c1ccc(-c2nc(-c3ccccc3)nc(-c3ccc4c(c3)-c3ccccc3C4(c3ccccc3)c3ccccc3N(c3ccc4ccc5ccccc5c4c3)c3cccc4oc5ccccc5c34)n2)cc1 ; c1ccc(-c2nc(N(c3ccccc3C3(c4ccccc4)c4ccccc4-c4ccccc43)c3cccc4sc5ccccc5c34)nc3c2ccc2ccccc23)cc1]

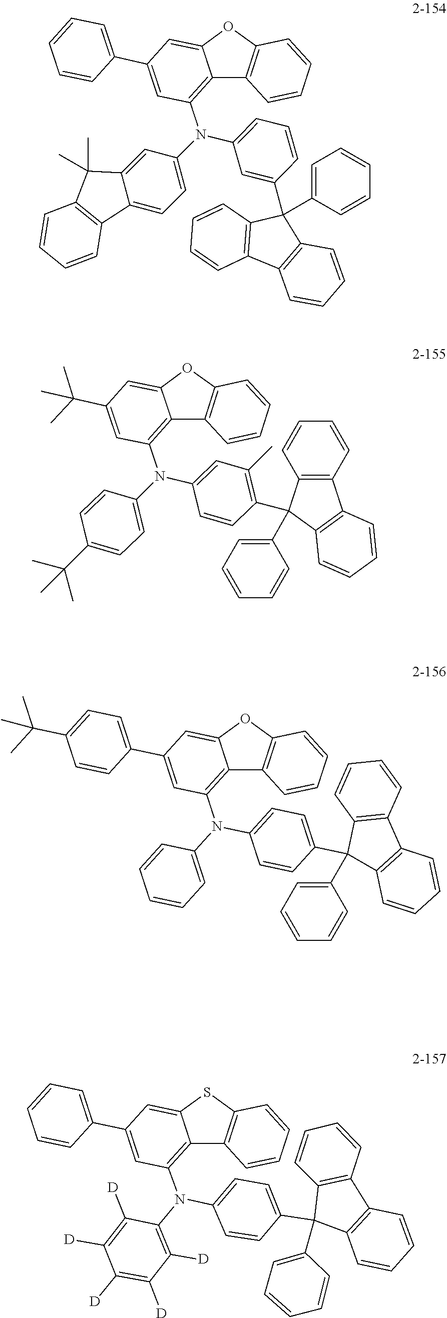

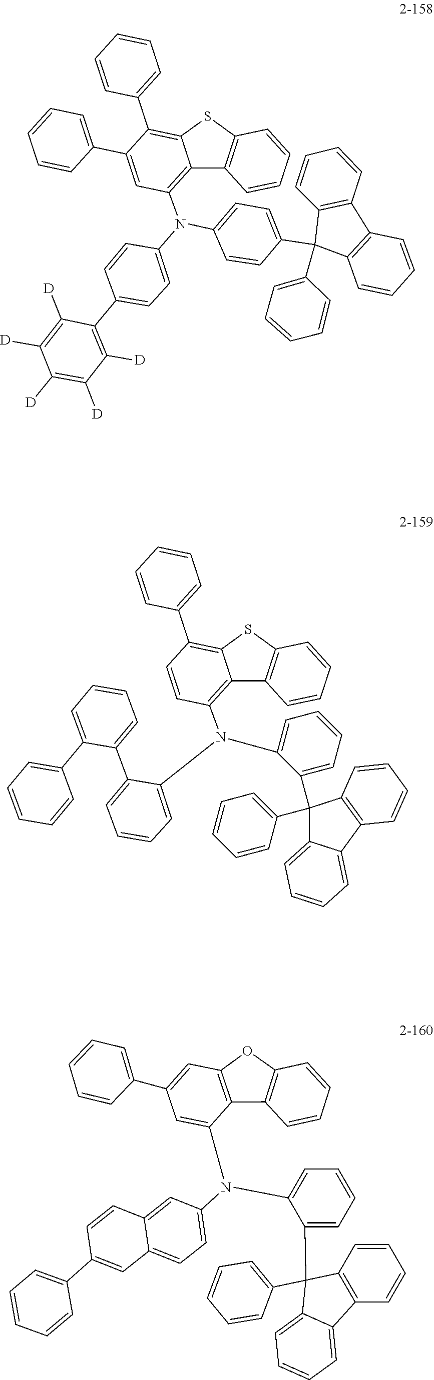 [SMILES: [2H]c1c([2H])c([2H])c(-c2ccc(N(c3ccc(C4(c5ccccc5)c5ccccc5-c5ccccc54)cc3)c3cc(-c4ccccc4)c(-c4ccccc4)c4sc5ccccc5c34)cc2)c([2H])c1[2H] ; c1ccc(-c2ccccc2-c2ccccc2N(c2ccccc2C2(c3ccccc3)c3ccccc3-c3ccccc32)c2ccc(-c3ccccc3)c3sc4ccccc4c23)cc1 ; c1ccc(-c2ccc3cc(N(c4ccccc4C4(c5ccccc5)c5ccccc5-c5ccccc54)c4cc(-c5ccccc5)cc5oc6ccccc6c45)ccc3c2)cc1]

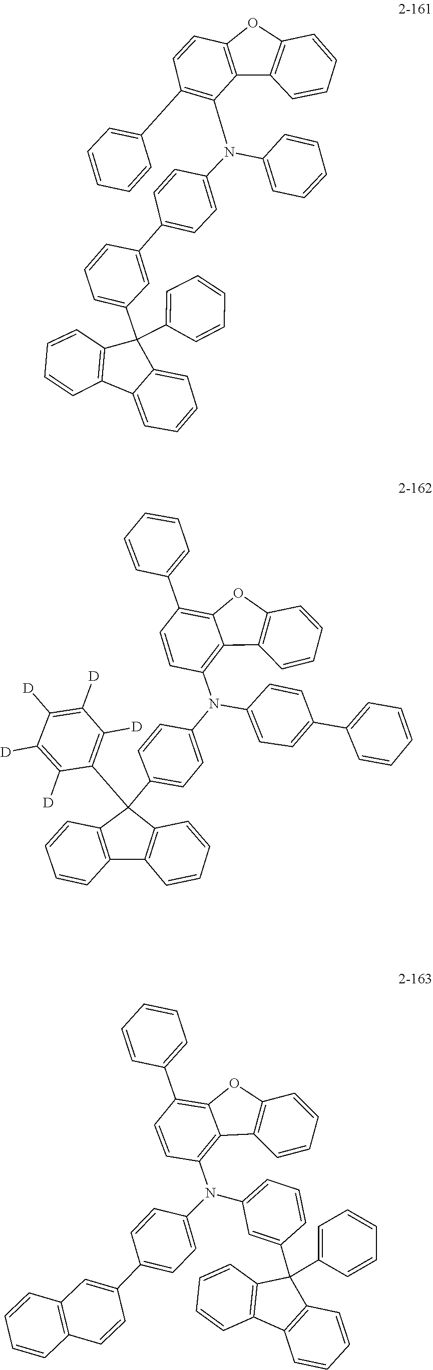 [SMILES: c1ccc(-c2ccc3oc4ccccc4c3c2N(c2ccccc2)c2ccc(-c3cccc(C4(c5ccccc5)c5ccccc5-c5ccccc54)c3)cc2)cc1 ; [2H]c1c([2H])c([2H])c(C2(c3ccc(N(c4ccc(-c5ccccc5)cc4)c4ccc(-c5ccccc5)c5oc6ccccc6c45)cc3)c3ccccc3-c3ccccc32)c([2H])c1[2H] ; c1ccc(-c2ccc(N(c3ccc(-c4ccc5ccccc5c4)cc3)c3cccc(C4(c5ccccc5)c5ccccc5-c5ccccc54)c3)c3c2oc2ccccc23)cc1]

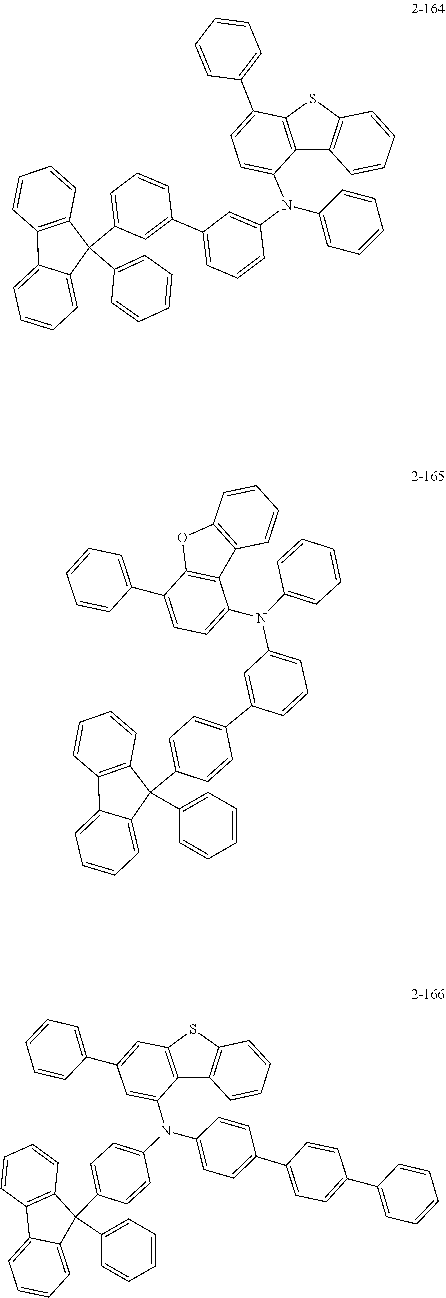 [SMILES: c1ccc(-c2ccc(N(c3ccccc3)c3cccc(-c4cccc(C5(c6ccccc6)c6ccccc6-c6ccccc65)c4)c3)c3c2sc2ccccc23)cc1 ; c1ccc(-c2ccc(N(c3ccccc3)c3cccc(-c4ccc(C5(c6ccccc6)c6ccccc6-c6ccccc65)cc4)c3)c3c2oc2ccccc23)cc1 ; c1ccc(-c2ccc(-c3ccc(N(c4ccc(C5(c6ccccc6)c6ccccc6-c6ccccc65)cc4)c4cc(-c5ccccc5)cc5sc6ccccc6c45)cc3)cc2)cc1]

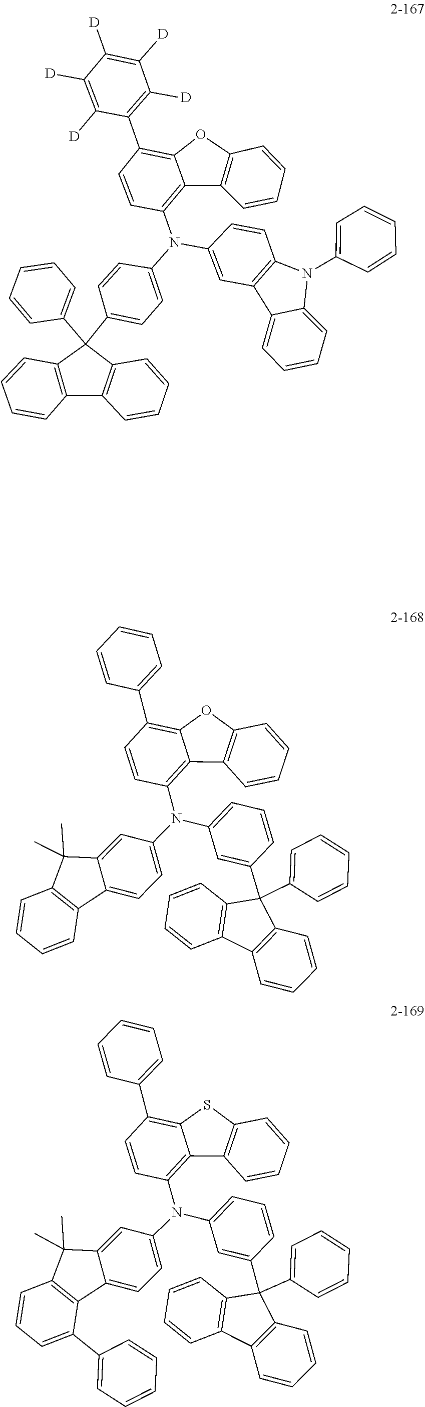 [SMILES: [2H]c1c([2H])c([2H])c(-c2ccc(N(c3ccc(C4(c5ccccc5)c5ccccc5-c5ccccc54)cc3)c3ccc4c(c3)c3ccccc3n4-c3ccccc3)c3c2oc2ccccc23)c([2H])c1[2H] ; CC1(C)c2ccccc2-c2ccc(N(c3cccc(C4(c5ccccc5)c5ccccc5-c5ccccc54)c3)c3ccc(-c4ccccc4)c4oc5ccccc5c34)cc21 ; CC1(C)c2cc(N(c3cccc(C4(c5ccccc5)c5ccccc5-c5ccccc54)c3)c3ccc(-c4ccccc4)c4sc5ccccc5c34)ccc2-c2c(-c3ccccc3)cccc21]

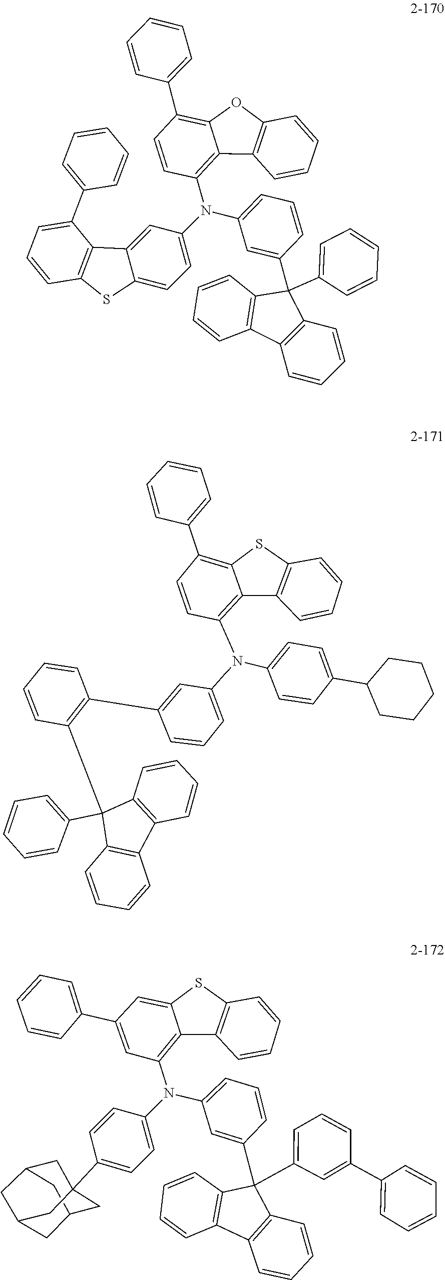 [SMILES: c1ccc(-c2ccc(N(c3cccc(C4(c5ccccc5)c5ccccc5-c5ccccc54)c3)c3ccc4sc5cccc(-c6ccccc6)c5c4c3)c3c2oc2ccccc23)cc1 ; c1ccc(-c2ccc(N(Cc3cccc(-c4ccccc4C4(c5ccccc5)c5ccccc5-c5ccccc54)c3)c3ccc(C4CCCCC4)cc3)c3c2sc2ccccc23)cc1 ; c1ccc(-c2cccc(C3(c4cccc(N(c5ccc(C67CC8CC(CC(C8)C6)C7)cc5)c5cc(-c6ccccc6)cc6sc7ccccc7c56)c4)c4ccccc4-c4ccccc43)c2)cc1]

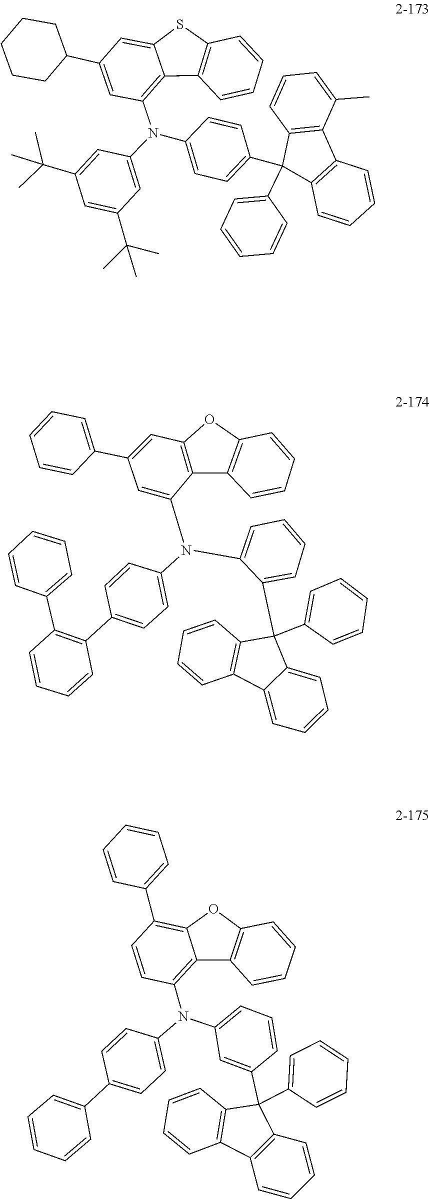 [SMILES: Cc1cccc2c1-c1ccccc1C2(c1ccccc1)c1ccc(N(c2cc(C(C)(C)C)cc(C(C)(C)C)c2)c2cc(C3CCCCC3)cc3sc4ccccc4c23)cc1 ; c1ccc(-c2cc(N(c3ccc(-c4ccccc4-c4ccccc4)cc3)c3ccccc3C3(c4ccccc4)c4ccccc4-c4ccccc43)c3c(c2)oc2ccccc23)cc1 ; c1ccc(-c2ccc(N(c3cccc(C4(c5ccccc5)c5ccccc5-c5ccccc54)c3)c3ccc(-c4ccccc4)c4oc5ccccc5c34)cc2)cc1]

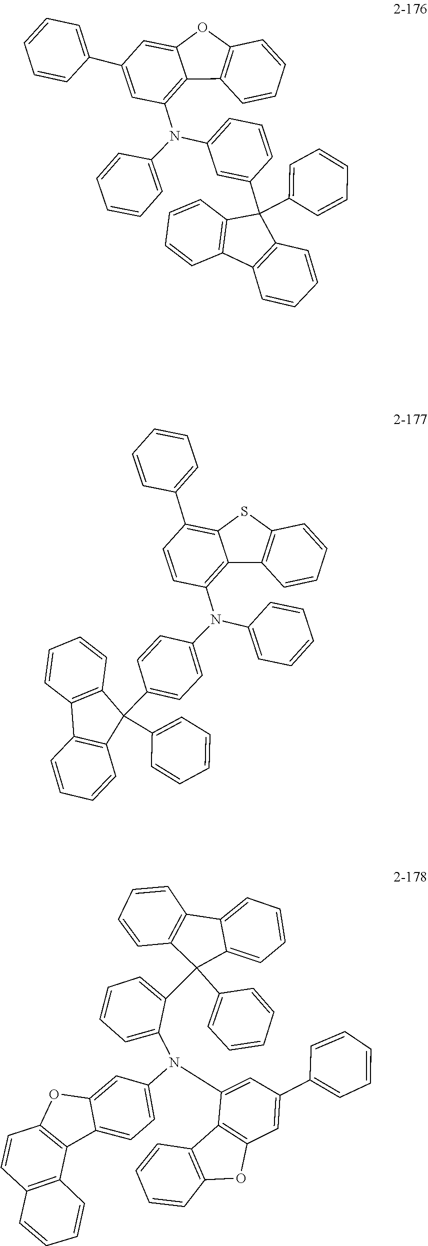 [SMILES: c1ccc(-c2cc(N(c3ccccc3)c3cccc(C4(c5ccccc5)c5ccccc5-c5ccccc54)c3)c3c(c2)oc2ccccc23)cc1 ; c1ccc(-c2ccc(N(c3ccccc3)c3ccc(C4(c5ccccc5)c5ccccc5-c5ccccc54)cc3)c3c2sc2ccccc23)cc1 ; c1ccc(-c2cc(N(c3ccc4c(c3)oc3ccc5ccccc5c34)c3ccccc3C3(c4ccccc4)c4ccccc4-c4ccccc43)c3c(c2)oc2ccccc23)cc1]

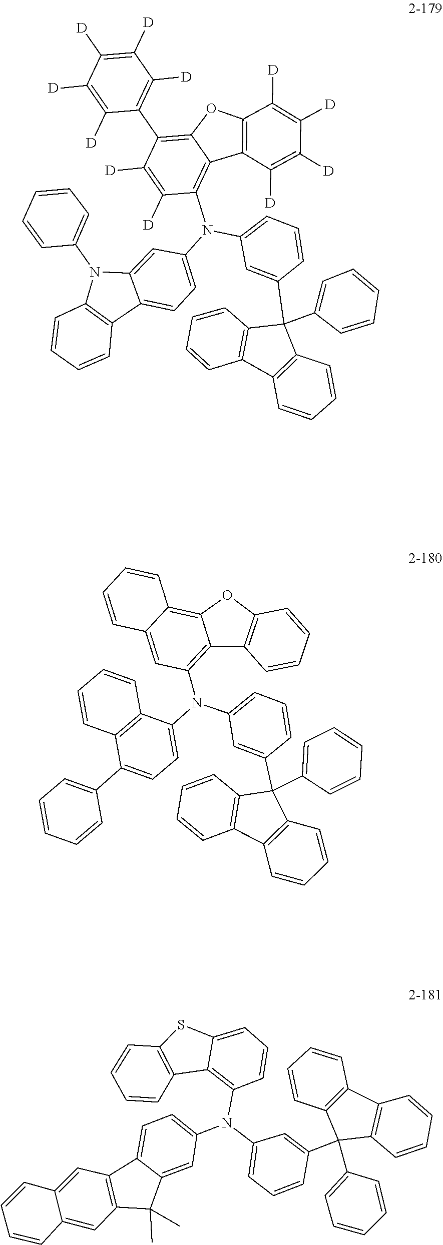 [SMILES: [2H]c1c([2H])c([2H])c(-c2c([2H])c([2H])c(N(c3cccc(C4(c5ccccc5)c5ccccc5-c5ccccc54)c3)c3ccc4c5ccccc5n(-c5ccccc5)c4c3)c3c2oc2c([2H])c([2H])c([2H])c([2H])c23)c([2H])c1[2H] ; c1ccc(-c2ccc(N(c3cccc(C4(c5ccccc5)c5ccccc5-c5ccccc54)c3)c3cc4ccccc4c4oc5ccccc5c34)c3ccccc23)cc1 ; CC1(C)c2cc(N(c3cccc(C4(c5ccccc5)c5ccccc5-c5ccccc54)c3)c3cccc4sc5ccccc5c34)ccc2-c2cc3ccccc3cc21]

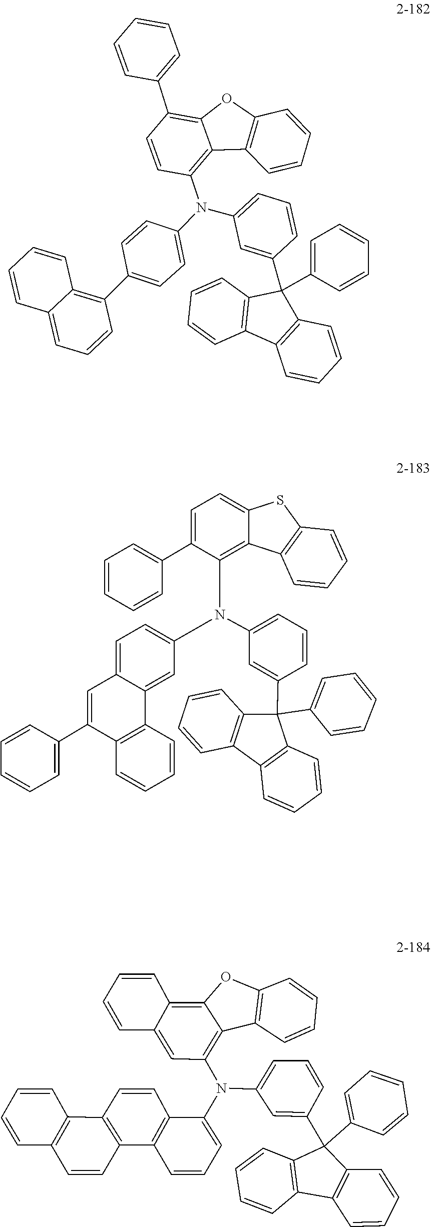 [SMILES: c1ccc(-c2ccc(N(c3ccc(-c4cccc5ccccc45)cc3)c3cccc(C4(c5ccccc5)c5ccccc5-c5ccccc54)c3)c3c2oc2ccccc23)cc1 ; c1ccc(-c2ccc3sc4ccccc4c3c2N(c2cccc(C3(c4ccccc4)c4ccccc4-c4ccccc43)c2)c2ccc3cc(-c4ccccc4)c4ccccc4c3c2)cc1 ; c1ccc(C2(c3cccc(N(c4cccc5c4ccc4c6ccccc6ccc54)c4cc5ccccc5c5oc6ccccc6c45)c3)c3ccccc3-c3ccccc32)cc1]

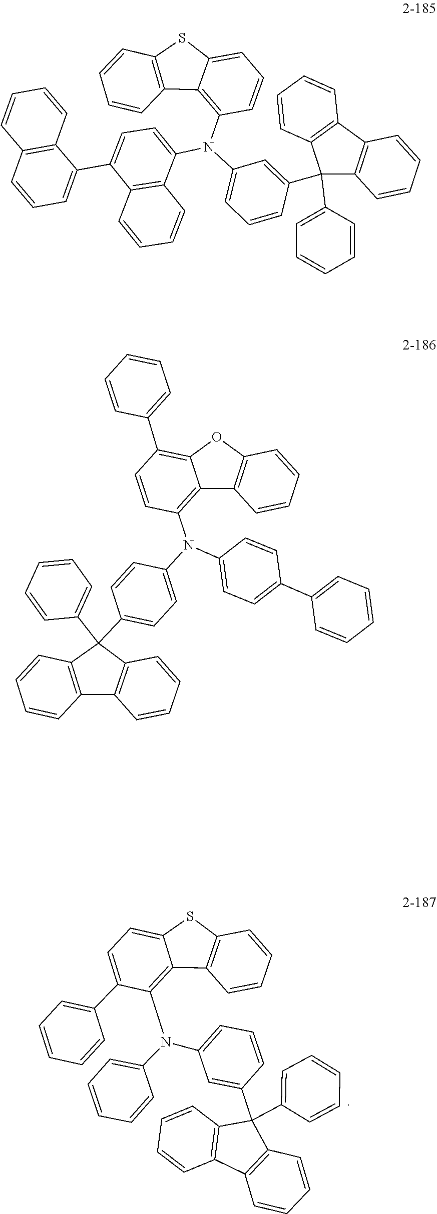 [SMILES: c1ccc(C2(c3cccc(N(c4ccc(-c5cccc6ccccc56)c5ccccc45)c4cccc5sc6ccccc6c45)c3)c3ccccc3-c3ccccc32)cc1 ; c1ccc(-c2ccc(N(c3ccc(C4(c5ccccc5)c5ccccc5-c5ccccc54)cc3)c3ccc(-c4ccccc4)c4oc5ccccc5c34)cc2)cc1 ; c1ccc(-c2ccc3sc4ccccc4c3c2N(c2ccccc2)c2cccc(C3(c4ccccc4)c4ccccc4-c4ccccc43)c2)cc1]

7. An organic electronic element comprising an anode; a cathode; an organic material layer formed between the anode and the cathode; wherein the organic material layer includes an emitting layer, an hole transport layer formed between the anode and the emitting layer; an emitting auxiliary layer or an electron blocking layer formed between the emitting layer and the hole transport layer, wherein the emitting auxiliary layer or the electron blocking layer comprises a compound represented by Formula (37) of claim 1.

8. A display device comprising the organic electronic element of claim 1; and a control part driving the display device;

9. A display device according to claim 8, wherein the organic electronic element is at least one of an OLED, an organic solar cell, an organic photo conductor (OPC), organic transistor (organic TFT) and an element for monochromic or white illumination.
